(19) United States
(12) Patent Application Publication
(10) Pub. No.: US 2005/0014767 A1
(43) Pub. Date: Jan. 20, 2005

(54) Benzoxazole, benzothiazole, and benzimidazole derivatives for the treatment of cancer and other diseases

(76) Inventors: Magnus Pfahl, Solana Beach, CA (US); Catherine Tachdjian, San Diego, CA (US); Torsten R. Wiemann, Encinitas, CA (US); Christopher N. Cow, San Diego, CA (US); Lyle W. Spruce, Chula Vista, CA (US); Andrea Fanjul Giachino, San Diego, CA (US); Allan A. Kaspar, San Diego, CA (US); James W. Zapf, San Diego, CA (US)

Correspondence Address: NEEDLE & ROSENBERG, P.C., SUITE 1000, 999 PEACHTREE STREET, ATLANTA, GA, 30309-3915, US

(21) Appl. No.: 10/769,725

(22) Filed: Jan. 29, 2004

Related U.S. Application Data
(60) Provisional application No. 60/443,426, filed on Jan. 29, 2003.

(51) Int. Cl. A61K 31/497; A61K 31/4439; A61K 31/423; A61K 31/4184

(52) U.S. Cl. 514/255.05; 514/337; 514/338; 514/375; 514/394; 544/405; 546/272.7; 548/215; 548/304.7

(57) ABSTRACT

The invention relates to certain compounds whose structures are shown below, and their pharmaceutically acceptable salts and prodrugs, and pharmaceutical compositions thereof, which are useful for treating treating diseases of uncontrolled cellular proliferation, including cancer. 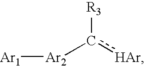 wherein:
a) Ar₁ has the structure: 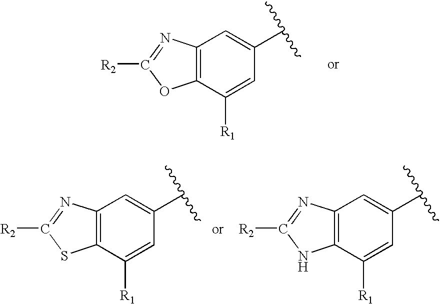 wherein
a) R₁ has the structure 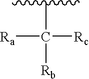
b) Ar₂ has the structure; 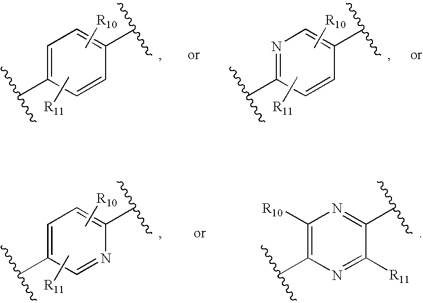
c) R₃ is hydrogen, or an alkyl radical; d) ----- represents a bond present or absent; and
   e) HAr has the formula

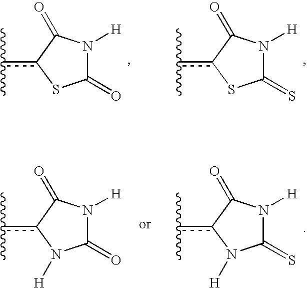

### RELATED APPLICATIONS

This application claims priority to the U.S. Provisional Application Ser. No. 60/443,426, filed Jan. 29, 2003, the entire disclosure of which application is hereby incorporated herein in its entirety by this reference.

### BACKGROUND OF THE INVENTION

Solid tumors are the leading cause of death attributable to cancers worldwide. Conventional methods of treating cancer include surgical treatments, the administration of chemotherapeutic agents, and recently immune based treatments, which typically involve the administration of an antibody or antibody fragment. Surgical treatments are generally only successful if the cancer is detected at an early stage, i.e., before the cancer has infiltrated major organs. Immune based treatments are subject to problems, including difficulty in targeting antibodies to desired sites, e.g., solid tumors, and host immune reactions to the administered antibody.

The usage of small molecule chemotherapeutics for the treatment of cancer has been one of the mainstream approaches. Ideally, anti-cancer chemotherapeutic agents selectively induce tumor cells to undergo the process of cellular suicide, termed apoptosis. Many of the chemotherapeutic treatments available for clinical application today are of limited usefulness and effectiveness because of their non-selective killing and/or toxicity to most cell types. Also, many tumor cells eventually become resistant against conventional chemotherapeutic agent, thus requiring treatment of such resistant tumors with new agents.

Antiestrogens and antiandrogens for the treatment/prevention of certain cancers are excellent examples of a class of small molecule ligands that function via their influence on nuclear receptor signaling pathways. Small molecules that are useful in the treatment of certain diseases were disclosed in U.S. patent application Ser. No. 09/655,460 filed Aug. 31, 2000, which is related to PCT International Publication WO 01/16122, published Mar. 8, 2001; in U.S. patent application Ser. No. 09/652,810 filed Aug. 31, 2000, and the related publication WO 01/16123, published Mar. 8, 2001; in U.S. patent application Ser. No. 10/094,142, filed Mar. 7, 2002, which is related to PCT International Publication WO 02/072009, published Sep. 19, 2002. The disclosures of WO 01/16122, WO 01/16123, and WO 02/072009, and their related United States patent applications are hereby incorporated herein by this reference in their entirety including their chemical structural disclosures, and their teachings of the biological activities of their compounds, and methods for their use as pharmaceutical compositions. Nevertheless, there is a continuing need for new anti-cancer chemotherapeutic agents that are both more effective, more specific, and less toxic that existing agents.

Apoptosis can be induced by the activation of cellular signaling pathways which lead to cell death. One specific cellular signaling pathway which can lead to apoptosis of cells involves the activation of JNK (Jun N-terminal Kinase), a protein kinase of the MAP-Kinase (Mitogen-Activated Protein Kinase) family. JNK proteins are activated by phosphorylation in response to diverse pro-apoptotic stimuli. Three genes encode JNK proteins, JNK-1, -2, and -3. These three genes give rise to 10 different isoforms of JNK. JNK-3 is highly expressed in neurons, whereas JNK-1 and -2 are ubiquitously expressed. Evidence for a role for JNK proteins in apoptosis comes from mice engineered to lack expression of specific JNK proteins. Mice lacking JNK-3 are resistant to excitatory stimulus-induced apoptosis of neurons. Cells from mice lacking both JNK-1 and -2 are resistant to stress-induced apoptosis, including death signals such as UV-irradiation and the translational inhibitor anisomycin. Activating the JNK pathway or sensitizing a tumor cell to the activation of the JNK pathway is one possible mechanism by which a chemotherapeutic agent can exert an anti-cancer effect. Activation of JNK is for instance induced by cisplatin and other anticancer agents. The activation of JNK is at least in part controlled by phosphatases in particular the dual specificity phosphatase MKP-1 (Sanchez-Perez et al, Oncogene (2000) 19, 5142-5152). Thus inhibition of MKP-1 by small molecule inhibitors provides a way of inducing JNK activation and apoptosis in cancer cells.

### SUMMARY OF THE INVENTION

The present invention relates to a series of substituted benzoxazole, benzothiazole, and benzimidazole heterocyclic compounds that unexpectedly exhibit potent activity for inducing the apoptosis of cancer cells, and accordingly show unexpectedly potent anti-cancer activity in vitro and/or in vivo. The substituted benzoxazole, benzothiazole, and benzimidazole heterocyclic compounds disclosed herein are useful in the treatment of diseases of uncontrolled proliferation, such as cancer and precancerous conditions, particularly those found in mammals, including humans. Therefore, methods of using the benzoxazole, benzothiazole, and benzimidazole compounds for the treatment of diseases of uncontrolled proliferative diseases are disclosed herein.

In another aspect, the inventions relate to pharmaceutical compositions for the treatment of diseases of uncontrolled cellular proliferation and cancers, the pharmaceutical compositions comprising one or more of the benzoxazole, benzothiazole, and benzimidazole compounds described herein as an admixture with one or more pharmaceutically acceptable carriers or excipients.

Other aspects of the invention relate to methods of synthesizing the substituted benzoxazole, benzothiazole, and benzimidazole compounds whose structures are described herein.

### BRIEF DESCRIPTION OF THE DRAWINGS

FIG. 1 shows one example synthetic pathway for the synthesis of the benzoxazole compounds of the invention.

FIG. 2 shows an alternative synthetic pathway for the synthesis of the benzoxazole compounds of the invention, and various methods for reacting aminophenol synthetic intermediates to provide variously substituted benzoxazole compounds.

FIG. 3a shows methods for the synthesis of 5-brominated benzoxazole synthetic precursors of the Ar₁ radicals of the compounds of the invention.

FIG. 3b shows methods for the synthesis of 5-brominated benzoxazole synthetic precursors of the Ar₁ radicals of the compounds of the invention.

FIG. 3c shows methods for the synthesis of benzoxazole precursor compounds comprising nitrogen substituted adamantyl radicals

FIG. 4a shows methods for the synthesis of synthetic precursors of the benzothiazole compounds of the invention.

FIG. 4b shows methods for the synthesis of synthetic precursors of the benzimidazole compounds of the invention.

FIG. 5 shows methods for elaborating certain carbonyl containing synthetic intermediates to form compounds of the invention comprising certain types of five membered heterocycles.

FIG. 6 shows methods for synthesizing heteroatom linked compounds of Formula (II).

FIG. 7 shows data on the effectiveness of certain compounds of the invention for killing non-small cell lung cancer cells in vitro, as a function of compound concentration, as described in Example 21.

FIG. 8 shows data on the effectiveness of certain compounds of the invention for killing breast cancer cells in vitro, as a function of compound concentration, as described in Example 21.

FIG. 9 shows data on the effectiveness of certain compounds of the invention for killing prostate cancer cells in vitro, as a function of compound concentration, as described in Example 21.

FIG. 10 shows data on the effectiveness of certain compounds of the invention for killing pancreatic cancer cells in vitro, as a function of compound concentration, as described in Example 21.

FIG. 11 shows data on the comparative activity compounds 1 and 2 of the invention for killing breast cancer cells in vitro, as compared to comparative compound 4, as described in Example 22.

FIG. 12 shows data on the comparative activity compounds 1 and 2 of the invention for killing pancreatic cancer cells in vitro, as compared to comparative compound 4, as described in Example 22.

FIG. 13 shows data on the comparative activity compounds 1 and 2 of the invention for killing lung cancer cells in vitro, as compared to comparative compound 4, as described in Example 22.

FIG. 14 shows data on the comparative activity compounds 1 and 2 of the invention for killing prostate cancer cells in vitro, as compared to comparative compound 4, as described in Example 22.

FIG. 15 shows the results of a Western Blot Assay for JNK protein phosphorylation in human lung cancer cells by compounds 1, 2, 11, and 12, as described in Example 23.

### DETAILED DESCRIPTION

The present invention relates to substituted benzoxazole, benzothiazole, and benzimidazole compounds that are useful, for example, to treat diseases of uncontrolled proliferation, for example for the treatment of cancers and precancerous conditions. The present invention can be understood more readily by reference to the following detailed description of preferred embodiments of the invention and the Examples included therein and to the Figures and their previous and following description. It is also to be understood that the terminology used herein is for the purpose of describing particular embodiments only and is not intended to be limiting.

Definitions

In the specification and Formulae described herein the following terms are hereby defined.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species.

The term “radical” as used in the specification and concluding claims, refers to a fragment, group, or substructure of a molecule described herein, regardless of how the molecule is prepared. For example, an adamantyl radical in a particular compound has the structure 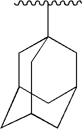
regardless of whether adamantane is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Inorganic radicals,” as the term is defined and used herein contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthamide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

“Organic radicals” as the term is defined and used herein contain one or more carbon atoms. An organic radical can have, for example, 1-26 carbon atoms, 1-18 carbon atoms, 1-12 carbon atoms, 1-6 carbon atoms, or 1-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The phrase “therapeutically effective amount” means an amount of a compound or combination of compounds that ameliorates, attenuates, or eliminates a particular disease or condition or prevents or delays the onset of a particular disease or condition.

The term “alkyl” denotes a radical containing a saturated, straight or branched hydrocarbon residue having from 1 to 18 carbons, or preferably 4 to 14 carbons, 5 to 13 carbons, or 6 to 10 carbons. An alkyl is structurally similar to a non-cyclic alkane compound modified by the removal of one hydrogen from the non-cyclic alkane and the substitution therefore with a non-hydrogen group or radical. Alkyl radicals can be branched or unbranched. Lower alkyl radicals have 1 to 4 carbon atoms. Examples of alkyl radicals include methyl, ethyl, n-propyl, iso-propyl, n-butyl, sec-butyl, t-butyl, amyl, t-amyl, n-pentyl and the like.

The term “substituted alkyl” denotes an alkyl radical analogous to the above definition that is substituted with one or more organic or inorganic substiuent radicals. In some embodiments, 1 or 2 organic or inorganic substiuent radicals are employed. In some embodiments, each organic substiuent radical comprises between 1 and 4, or between 5 and 8 carbon atoms. Suitable organic and inorganic substiuent radicals include but are not limited to hydroxyl, halogens, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, heteroaryl, substituted heteroaryl, aryl or substituted aryl. When more than one substiuent group is present then they can be the same or different.

The term “alkenyl” denotes an alkyl radical as defined above, having 1 to 18 carbons, or preferably 4 to 14 carbons, 5 to 13 carbons, or 6 to 10 carbons which further contains a carbon-carbon double bond. Examples of alkenyl radicals include but are not limited to vinyl, allyl, 2-butenyl, 3-butenyl, 2-pentenyl, 4-methyl-penten-2-yl, 3-pentenyl, 4-methyl-penten-3-yl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl, and like residues. The term “alkenyl” includes dienes and trienes and other polyunsaturated compounds. The alkenyl radical can exist as E or Z stereoisomers or as a mixture of E or Z stereoisomers. When more than one double bond is present, such as a diene or triene, each double bond can independently exist as E or Z stereoisomers or as a mixture of E or Z stereoisomers with respect to other double bond present in the alkenyl radical.

The term “substituted alkenyl” denotes a alkenyl radical of the above definition that is further substituted with one or more substituent inorganic or organic radicals, which can include but are not limited to halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. In some embodiments, 1 or 2 organic or inorganic substituent radicals are employed. In some embodiments, each organic substituent radical comprises between 1 and 4, or between 5 and 8 carbon atoms. When more than one group is present then they can be the same or different.

The term “alkynyl” denotes a radical containing a straight or branched chain of having 1 to 18 carbons, or preferably 4 to 14 carbons, 5 to 13 carbons, or 6 to 10 carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and like residues. The term “alkynyl” includes di- and tri-ynes.

The term “substituted alkynyl” denotes a alkynyl of the above definition that is substituted with one or more organic or inorganic radicals, that can include halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy residues.

The term “cycloalkyl” denotes a radical containing 1 to 18 carbons, or preferably 4 to 14 carbons, 5 to 10 carbons, or 5 to 6 carbons, such as cyclopropyl, cyclobutyl, cyclopentyl, cyclopentyl, cyclohexyl, cycloheptyl, decahydronapthyl, adamantyl, and like residues.

The term “substituted cycloalkyl” denotes a cycloalkyl as defined above that is further substituted with one or more organic or inorganic groups that can include halogen, alkyl, substituted alkyl, hydroxyl, alkoxy, substituted alkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkyl is substituted with more than one group, they can be the same or different.

The term “cycloalkenyl” denotes a cycloalkyl radical further comprising at least one carbon-carbon double bond, including cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl, and like radicals.

The term “substituted cycloalkenyl” denotes a cycloalkenyl residues as defined above further substituted with one or more groups selected from halogen, alkyl, hydroxyl, alkoxy, substituted alkoxy, haloalkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkenyl is substituted with more than one group, they can be the same or different.

The term “alkoxy” as used herein denotes a radical alkyl, defined above, attached directly to a oxygen to form an ether residue. Examples include methoxy, ethoxy, n-propoxy, iso-propoxy, n-butoxy, t-butoxy, iso-butoxy and the like.

The term “substituted alkoxy” denotes a alkoxy radical of the above definition that is substituted with one or more groups, but preferably one or two substituent groups including hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When more than one group is present then they can be the same or different.

The term “mono-substituted amino” denotes an amino (—NH₂) group substituted with one group selected from alkyl, substituted alkyl or arylalkyl wherein the terms have the same definitions found throughout.

The term “di-substituted amino” denotes an amino substituted with two radicals that can be same or different selected from aryl, substituted aryl, alkyl, substituted alkyl or arylalkyl wherein the terms have the same definitions found throughout. Some examples include dimethylamino, methylethylamino, diethylamino and the like.

The term “haloalkyl” denotes a alkyl radical, defined above, substituted with one or more halogens, preferably fluorine, such as a trifluoromethyl, pentafluoroethyl and the like.

The term “haloalkoxy” denotes a haloalkyl, as defined above, that is directly attached to an oxygen to form a halogenated ether residue, including trifluoromethoxy, pentafluoroethoxy and the like.

The term “acyl” denotes a radical of the formula —C(O)—R that comprises a carbonyl (C═O) group, wherein the R radical is an organic radical having a carbon atom bonded to the carbonyl group. Acyl radicals contain 1 to 8 or 1 to 4 carbon atoms. Examples of acyl radicals include but are not limited to formyl, acetyl, propionyl, butanoyl, iso-butanoyl, pentanoyl, hexanoyl, heptanoyl, benzoyl and like radicals.

The term “acyloxy” denotes a radical containing 1 to 8 carbons of an acyl group defined above directly attached to an oxygen such as acetyloxy, propionyloxy, butanoyloxy, iso-butanoyloxy, benzoyloxy and the like.

The term “aryl” denotes an unsaturated and conjugated aromatic ring radical containing 6 to 18 ring carbons, or preferably 6 to 12 ring carbons. Many aryl radicals have at least one six-membered aromatic “benzene” radical therein. Examples of such aryl radicals include phenyl and naphthyl.

The term “substituted aryl” denotes an aryl ring radical as defined above that is substituted with or fused to one or more organic or inorganic substituent radicals, which include but are not limited to a halogen, alkyl, substituted alkyl, haloalky, hydroxyl, cycloalkyl, substituted cycloalkyl, cycloalkenyl, substituted cycloalkenyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring radical, wherein the terms are defined herein. Substituted aryl radicals can have one, two, three, four, five, or more substituent radicals. The substituent radicals can be not be of unlimited size or molecular weight, and each organic radical can comprise 15 or fewer, 10 or fewer, or 4 or fewer carbon atoms unless otherwise expressly contemplated by the claims

The term “heteroaryl” denotes an aryl ring radical as defined above, wherein at least one of the carbons of the aromatic ring has been replaced with a heteroatom, which include but are not limited to nitrogen, oxygen, and sulfur atoms. Heteroaryl radicals include 6 membered aromatic ring radicals, and can also comprise 5 or 7 membered aromatic rings, or bicyclic or polycyclic heteroaromatic rings as well. Examples of heteroaryl radicals include pyridyl, bipyridyl, furanyl, and thiofuranyl residues. Further examples of heteroaryl residues which can be employed in the chemical structures of the invention include but are not limited to the residues exemplified below: 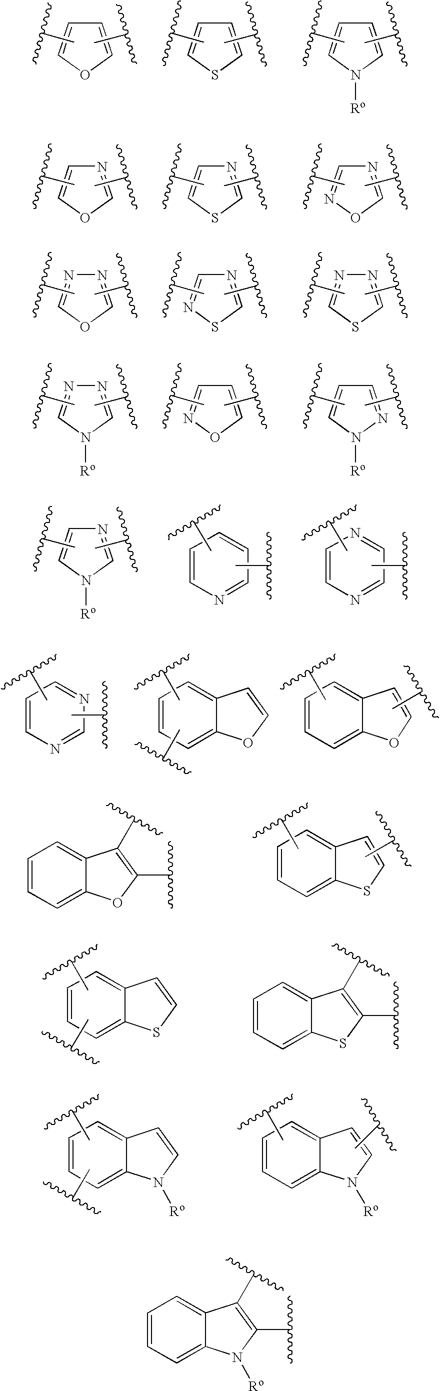
wherein R^{o} can be hydrogen, alkyl, substituted alkyl, aryl, substituted aryl, and the like. It is to be understood that the heteroaryl radicals can optionally be substituted with one or more organic or inorganic substituent radicals bound to the carbon atoms of the heteroaromatic rings, as described hereinabove for substituted aryl radicals. Substituted heteroaryl radicals can have one, two, three, four, five, or more substituent organic or inorganic radicals, in a manner analogous to the substituted aryl radicals defined herein. The substituent radicals cannot be of unlimited size or molecular weight, and each organic substituent radical can comprise 15 or fewer, 10 or fewer, or four or fewer carbon atoms unless otherwise expressly contemplated by the claims.

The term “halo,” “halide,” or “halogen” refers to a fluoro, chloro, bromo or iodo atom or ion.

The term “thioalkyl” denotes a sulfide radical containing 1 to 8 carbons, linear or branched. Examples include methylsulfide, ethyl sulfide, isopropylsulfide and the like.

The term “thiohaloalkyl” denotes a thioalkyl radical substituted with one or more halogens. Examples include trifluoromethylthio, 1,1-difluoroethylthio, 2,2,2-trifluoroethylthio and the like.

The term “carboalkoxy” refers to an alkyl ester of a carboxylic acid, wherein alkyl has the same definition as found above. Examples include carbomethoxy, carboethoxy, carboisopropoxy and the like.

The term “alkylcarboxamide” denotes a single alkyl group attached to the amine of an amide, wherein alkyl has the same definition as found above. Examples include N-methylcarboxamide, N-ethylcarboxamide, N-(iso-propyl)carboxamide and the like. The term “substituted alkylcarboxamide” denotes a single “substituted alkyl” group, as defined above, attached to the amine of an amide.

The term “dialkylcarboxamide” denotes two alkyl or arylalkyl groups that are the same or different attached to the amine of an amide, wherein alkyl has the same definition as found above. Examples of a dialkylcarboxamide include N,N-dimethylcarboxamide, N-methyl-N-ethylcarboxamide and the like. The term “substituted dialkylcarboxamide” denotes two alkyl groups attached to the amine of an amide, where one or both groups is a “substituted alkyl”, as defined above. It is understood that these groups can be the same or different. Examples include NN-dibenzylcarboxamide, N-benzyl-N-methylcarboxamide and the like.

The term “organoamide” denotes an acyl radical attached to an amine or monoalkylamine, wherein the term acyl has the same definition as found above. Examples of “alkylamide” include acetamido, propionamido and the like.

The term “heterocycle” or “heterocyclic”, as used in the specification and concluding claims, refers to a radical having a closed ring structure comprising 3 to 10 ring atoms, in which at least one of the atoms in the ring is an element other than carbon, such as, for example, nitrogen, sulfur, oxygen, silicon, phosphorus, or the like. Heterocyclic compounds having rings with 5, 6, or 7 members are common, and the ring can be saturated, or partially or completely unsaturated. The heterocyclic compound can be monocyclic, bicyclic, or polycyclic. Examples of heterocyclic compounds include but are not limited to pyridine, piperidine, thiophene, furan, tetrahydrofuran, and the like. The term “substituted heterocyclic” refers to a heterocyclic radical as defined above having one or more organic or inorganic substituent radicals bonded to one of the ring atoms.

The term “carboxy”, as used in the specification and concluding claims, refers to the —C(O)OH radical that is characteristic of carboxylic acids. The hydrogen of the carboxy radicals is often acidic and (depending on the pH) often partially or completely dissociates, to form an acid H⁺ ion and a carboxylate anion (—CO₂⁻), wherein the carboxylate anion is also sometimes referred to as a “carboxy” radical.

The term “nitrile”, as used in the specification and concluding claims, refers to a compound having a —CN substituent radical wherein the carbon is triply bonded to the nitrogen atom.

The term “alkylsilyloxy”, as used in the specification and concluding claims, refers to a radical of the formula —O—SiR₁R₂R₃ wherein the R₁, R₂, and R₃ groups are independently hydrogen or organic radicals, wherein the organic radicals preferably contain from one to ten carbon atoms.

The term “alkylene” as used herein refers to a difunctional saturated branched or unbranched hydrocarbon chain containing from 1 to 36 carbon atoms, and includes, for example, methylene (—CH₂—), ethylene (—CH₂—CH₂—), propylene (—CH₂—CH₂(CH₃)—), 2-methylpropylene [—CH₂—CH(CH₃)—CH₂—], hexylene [—(CH₂)₆-] and the like. “Lower alkylene” refers to an alkylene group of from 1 to 6, more preferably from 1 to 4, carbon atoms.

The term “cycloalkylene” as used herein refers to a cyclic alkylene group, typically a 5- or 6-membered ring.

The term “arylalkyl” defines an alkylene as described above which is substituted with an aryl group that can be substituted or unsubstituted as defined above. Examples of an “arylalkyl” include benzyl, phenethylene and the like.

The Compounds of The Invention

The compounds of the invention relate to compounds of the Formulas (I) or (II): 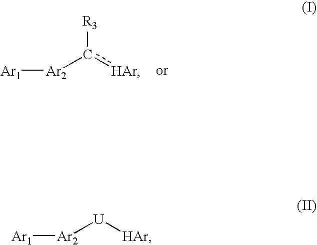
wherein:
a) Ar₁ has the structure: 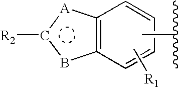
wherein
   i) R₁ is hydrogen, an inorganic radical, or an organic radical;
   ii) R₂ is hydrogen, an inorganic radical, or a organic radical;
   iii) A and B are independently selected from the group consisting of —O—, —N—, —NR₄—, and —S—, provided at least one of A or B is —N—, and R₄ is hydrogen or an organic radical, and C is a carbon atom;
b) Ar₂ is an aryl, a substituted aryl, a heteroaryl or a substituted heteroaryl radical;
c) R₃ is hydrogen, halogen, hydroxy, or an organic radical;
d) U is a heteroatomic linking radical selected from the group consisting of —NR₃—, —O—, —S—, —SO—, and —SO₂—;
(d) ----- represents a bond present or absent;
(e) HAr has the formula: 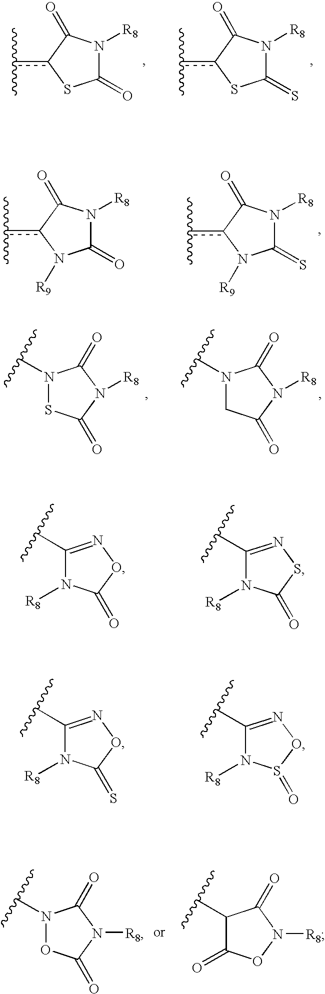
wherein R₈ and R₉ are independently selected from the group consisting of hydrogen or an organic radical;
or a pharmaceutically acceptable salt thereof.

The more detailed structural features of some embodiments of the above compounds of the invention will now be disclosed and described.

The compounds of the invention comprise Ar₁ radicals having five-membered oxazole, thiazole, or imidazole heterocyclic rings fused to a substituted benzene ring, so as to form corresponding benzoxazole, benzothiazole, or benzimidazole fused heterocyclic rings. The benzene ring is also bonded to the Ar₂ radical and to an R₁ substituent radical. The five-membered oxazole, thiazole, or imidazole ring can be fused to the benzene ring in any geometrical orientation (ortho, meta, or para) relative to the bonds to the Ar₂ and/or optional R₁ radicals, as shown below:

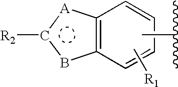

The A and B atoms are ring heteroatoms that can be independently selected from —O—, —S—, —N—, and —NR₄—, with the proviso that at least one of A or B is —N—, wherein R₄ is hydrogen or an organic radical, and C is a carbon atom. In some embodiments, R₄ is an organic radical comprising 1 to 4 carbon atoms, and in other embodiments R₄ is an alkyl or haloalkyl radical comprising 1 to 4 carbon atoms.

Because five membered oxazole, thiazole, or imidazole rings are heteroaromatic, and must contain both an unsubstituted nitrogen atom and a carbon atom bearing the R₂ substituent, the general structure of the Ar₁ radical can also be represented by the following formula: 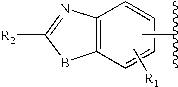
wherein B is selected from —O—, —S—, and —NR₄.

Examples of possible geometrical isomers of the Ar₁ radicals include the structures shown below:

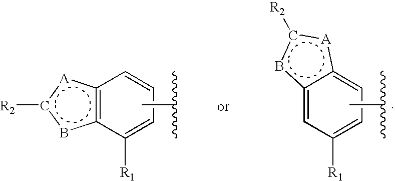

If one of A or B is —O—, and the other of A or B is —N—, an Ar₁ radical comprising a benzoxazole ring results. Examples of Ar₁ radicals that are benzoxazole radicals include the radicals shown below:

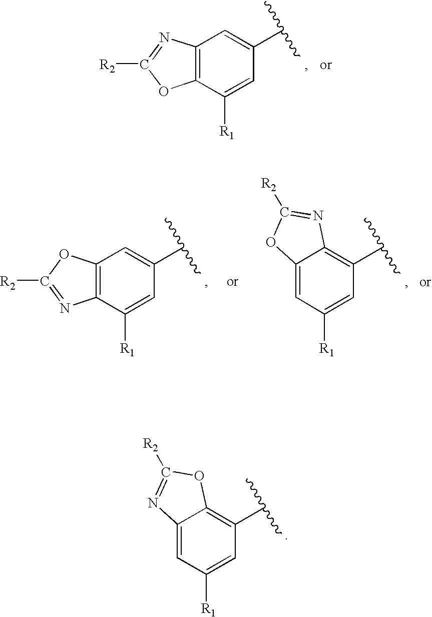

If one of A or B is —S—, and the other of A or B is —N—, an Ar₁ radical comprising a benzothiazole ring results. Examples of Ar₁ radicals comprising benzothiazoles include the radicals shown below:

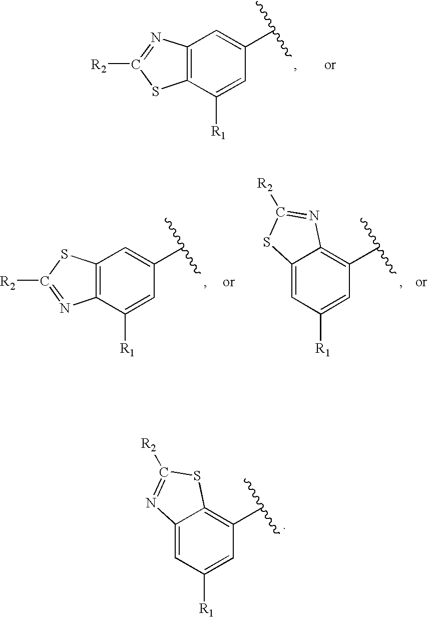

If one of A or B is —N—, and the other of A or B is —NR₄—, an Ar₁ radical comprising a benzimidazole ring results. Examples of Ar₁ radicals comprising benzimidazoles include the radicals shown below:

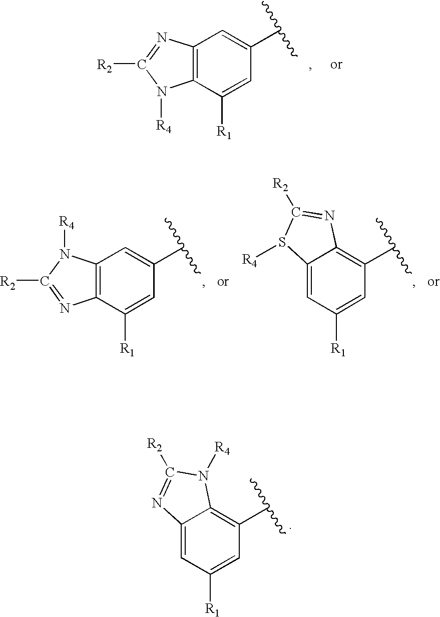

In many embodiments relating to Ar₁ radicals comprising benzimidazole rings, the R₄ group is hydrogen, resulting in benzimidazole rings that include those shown below, which those of ordinary skill in the art understand to be tautomers.

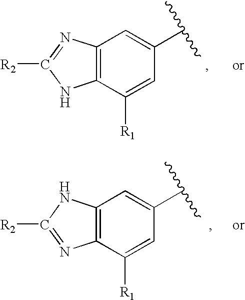

It has been found that, for at least for some strains of cancer cells, certain geometrical isomers for the Ar₁ radical can be related to better than average biological and/or anti-cancer activity, so that in some embodiments, the Ar₁ radicals have the structure:

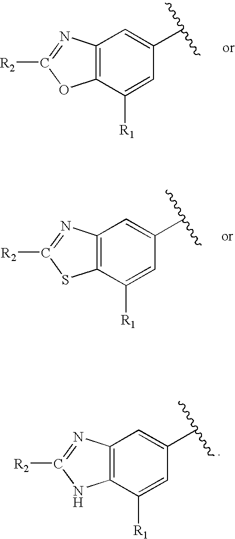

The benzene ring of the Ar₁ radical can also have an optional R₁ substituent, which can be selected from hydrogen, an inorganic radical, or an organic radical. The benzoxazole, benzothiazole, or benzimidazole rings also comprise a carbon atom having an R₂ substituent, which can also be selected from hydrogen, an inorganic radical, or an organic radical.

Although not wishing to be bound by theory, the compounds of the invention, including the Ar₁ radical together with the R₁ and R₂ substituent radicals can be selected so that the Ar₁ radical has a geometry, size, and polarity that is suitable to allow the compounds of the invention to interact with and substantially fill, yet fit within the binding regions of the target biological molecules, so as to contribute to the effective binding of the compounds to the binding sites in the biological target molecules, which are believed to be involved in JNK activation pathways. Therefore, in some embodiments, the Ar₁ radical, together with its substituent R₁ and R₂ radicals comprises from 7 to 30 carbon atoms, or from 8 to 25 carbon atoms, from 9 to 20 carbon atoms, or from 10 to 18 carbon atoms.

The R₁ substituent can be selected from hydrogen, an inorganic radical, or an organic radical. Suitable inorganic radicals, as defined elsewhere herein, include but are not limited to halogens (fluorine, chlorine, bromine, or iodine), hydroxyl, amino, nitro, and thiol, sulfate, phosphate, and like radicals known to those of ordinary skill in the art.

R₁ can be and often is an organic radical, as defined elsewhere herein. The organic radical must comprise at least one carbon atom, and may optionally comprise heteroatoms. In some embodiments, R₁ comprises from 1 to 18 carbon atoms, from 3 to 12 carbon atoms, or from 4 to 10 carbon atoms.

In some embodiments, R₁ is selected from an alkyl, a haloalkyl, a cycloalkyl, a cycloalkenyl, a heterocyclic, a heteroaryl, a substituted heteroaryl, an aryl or a substituted aryl radical. In some embodiments, R₁ is selected from an acyl, ketoxime, alkoxy, substituted alkoxy, phenoxy, substituted phenoxy, haloalkoxy, monosubstituted amino, disubstituted amino, thioalkyl, alkylsulfonyl, alkylsulfinyl, carboxy, carboalkoxy, carboaryloxy, alkylcarboxamide, dialkylcarboxamide, alkylamide, or arylamide radical. Examples of such R₁ groups include those illustrated below:

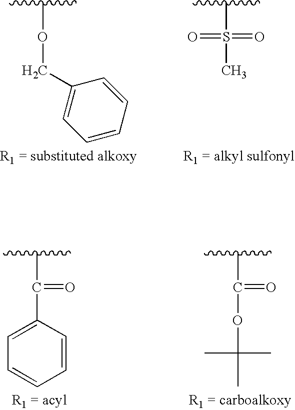

In further embodiments, R₁ is selected from a heteroaryl, a substituted heteroaryl, an aryl or a substituted aryl radical, or an aralkyl.

In some embodiments, R₁ has the formula 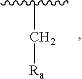
wherein Rₐ is an inorganic radical or organic radical comprising 3 to 12 carbon atoms.

In some embodiments, R₁ is selected from a cycloalkyl, a substituted cycloalkyl, a heterocyclic, or a substituted heterocyclic radical. Such cycloalkyl or heterocyclic radicals can be polycyclic, as will be further described below.

In certain embodiments of the invention, the anti-cancer activity of the compounds of the invention is substantially and unexpectedly improved if the R₁ radical is a “bulky” (i.e. sterically demanding) substituent radical. Those of ordinary skill in organic chemistry are aware of many types of bulky substituent radicals. One type of bulky substituent radical has the following formula; 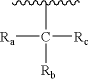
wherein Rₐ, R_{b}, and R_{c} are independently selected from hydrogen, or an inorganic or organic radical as defined elsewhere herein, with the proviso that no more than one of Rₐ, R_{b}, and R_{c} are hydrogen, so that the bulky substituent radical has a branched central carbon atom.

In some embodiments, one of Rₐ, R_{b} and R_{c} is a hydrogen atom, and two of Rₐ, R_{b}, and R_{c} are organic radicals. In some embodiments, the two organic radicals are independently selected from an alkyl, substituted alkyl, cycloalkyl, substituted alkyl, heterocyclic or substituted heterocyclic radical. Moreover, in some embodiments, at least two of Rₐ, R_{b} and R_{c} together form a cycloalkyl, substituted cycloalkyl, heterocyclic or substituted heterocyclic ring radical.

Examples of branched substituent radicals wherein one of Rₐ, R_{b} and R_{c} is a hydrogen atom and two of Rₐ, R_{b} and R_{c} are organic radicals include the isopropyl, 2-methylpropyl, cyclopentyl, and cyclohexyl radicals shown below.

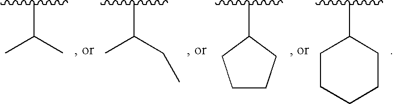

In some embodiments none of Rₐ, R_{b}, and R_{c} are hydrogen. In some such embodiments Rₐ, R_{b}, and R_{c} are independently alkyls that each comprise 1 to 4 carbon atoms, and therefore a tertiary carbon atom is bonded to the benzene ring or Ar₁. Examples of such tertiary alkyl substituents include radicals such as:

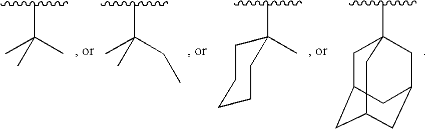

As illustrated above, two or three of the Rₐ, R_{b}, and R_{c} radicals of the branched radical can be bonded together to form cyclic, bicyclic, polycyclic, heterocyclic, alicyclic, aryl, or heteroaryl rings. The Rₐ, R_{b}, and R_{c} radicals can in some embodiments be substituted with additional organic or inorganic substituent radicals. Examples of such branched radicals having cyclic radicals include:

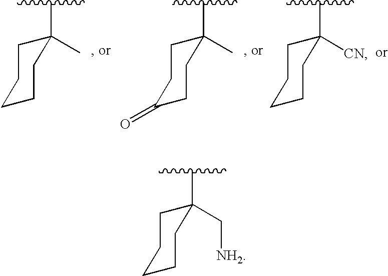

The R₁ radical can be a substituted “adamantyl” radical of the Formula (Villa): 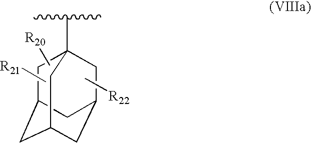
wherein R₂₀, R₂₁ and R₂₂ can be independently selected from hydrogen, an inorganic radical, or an organic radical at any position on the adamantyl radical. In some embodiments, R₂₀, R₂₁ and R₂₂ are independently selected from hydrogen, halogen, alkyl, hydroxy, carboxyl, alkylcarboxamide or dialkylcarboxamide radicals. In one embodiment R₁ is a substituted cycloalkyl of Formula (VIIIa) wherein R₂₀, R₂₁ and R₂₂ are hydrogen, such that the substituted cycloalkyl is an unsubstituted adamantyl radical of Formula (VIIIb):

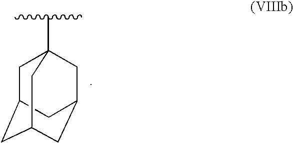

In another embodiment the branched substituent radical is a substituted adamantyl radical of Formula (VIIIa) wherein R₂₀ is a fluorine, to provide a radical of Formula (VIIc):

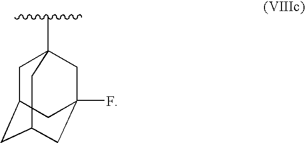

Some embodiments of the invention relate to compounds of Formula (I) wherein the branched substituent radical is a substituted heterocyclic radical of the Formula (VIId): 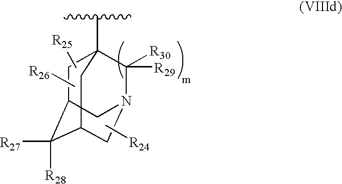
wherein:
   m is 0 or 1;
   R₂₄, R₂₅ and R₂₆ can be attached to any carbon on the substituted heterocyclic radical except for the carbons bearing R₂₇ and R₂₈ or R₂₉ and R₃₀ and are independently hydrogen, halogen, alkyl, hydroxy, carboxyl, alkylcarboxamide or dialkylcarboxamide;
   R₂₇ and R₂₈ are independently hydrogen, halogen, or hydroxy; or R₂₇ and R₂₈ together form a carbonyl radical;
   R₂₉ and R₃₀ are independently hydrogen; or R₂₉ and R₃₀ together form a carbonyl radical.

In one embodiment the branched substituent radical is a substituted heterocyclic radical of Formula (Vied) wherein m is 0; R₂₄, R₂₅ and R₂₆ are hydrogen; R₂₇ and R₂₈ are each hydrogen or R₂₇ and R₂₈ together form a carbonyl radical of the following formulas:

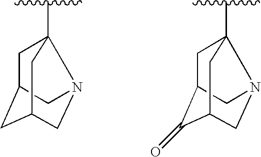

In one embodiment, the branched radical is a substituted heterocyclic radical of Formula (VIId) wherein m is 1, R₂₄ and R₂₅ are independently an alkyl, R₂₆ is hydrogen and R₂₇ and R₂₈ are each a hydrogen or R₂₇ and R₂₈ together form a carbonyl of the for following formulas:

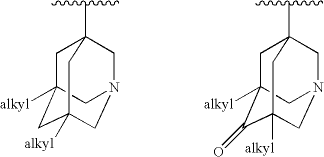

In one embodiment, the branched substituent radical is a substituted heterocyclic radical of Formula (VIIId) wherein m is 1; R₂₄, R₂₅ and R₂₆ are hydrogen; R₂₇ and R₂₈ are hydrogen or R₂₇ and R₂₈; and R₂₉ and R₃₀ together form a carbonyl of the following formulas:

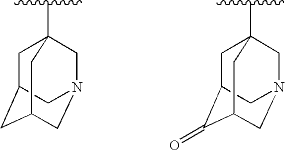

In certain embodiments, R₁ is a t-butyl, a 2-methylpropyl, a phenyl, a 2-pyridyl, a 3-pyridyl, a 4-pyridyl, a 1-alkylcyclohexyl, azaadamantyl, azaadamantone-yl or an adamantyl radical.

For the Ar₁ radicals comprising benzoxazole, benzothiazole, and benzimidazole ring radicals, beneficial results can often be obtained if R₁ is one of the bulky and/or branched radicals as illustrated by the structures below; 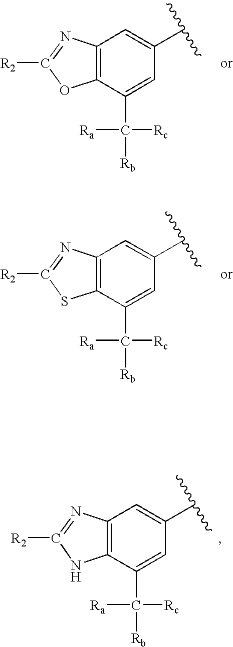
wherein Rₐ, R_{b}, and R_{c} can be defined as in any of the embodiments described above.

For example, in some embodiments, compounds containing Ar₁ radicals of the following structures can be desirable;

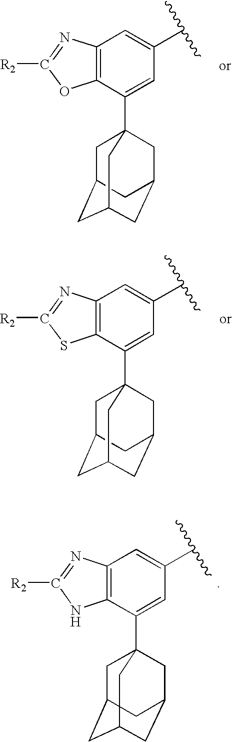

In other embodiments, Ar₁ radicals comprising benzoxazole, benzothiazole, and benzimidazole ring radicals include

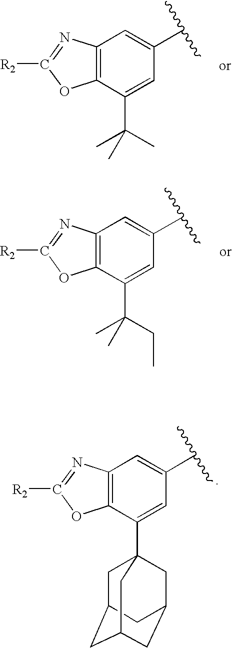

Ar₁ also has an R₂ substituent radical bonded to the carbon atom of the benzoxazole, benzothiazole, or benzimidazole rings that can be hydrogen, an inorganic radical, or a organic radical, as defined elsewhere herein. In some embodiments, R₂ is an inorganic radical selected from hydrogen, —SH, —NH₂ (amino), or the halogens. In some embodiments, R₂ is an organic radical having from one 1 to 7 carbon atoms, which may optionally comprise one to three heteroatoms selected from the group consisting of O, S, N, and halogens. In related embodiments, R₂ is selected from an alkoxy, carboalkoxy, haloalkyl, sulfhydril, amino, disubstituted amino, —CH₂—S—R′, —NH(CO)—R′, —NH—C(NH)NH₂, —CH₂—NHR′, —CH₂—NR′R″, and 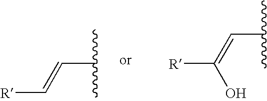
wherein R′ and R″ are independently selected lower alkyls.

The compounds of the invention comprise Ar₂ radicals bound to both Ar₁ and to a bridging radical that links Ar₂ to the HAr heterocycles. The Ar₂ radicals can be an aryl, a substituted aryl, a heteroaryl or a substituted heteroaryl radical, as defined elsewhere herein. Although again not wishing to be bound by theory, it is believed that the Ar₂ radical and any of its substituent radicals should be selected to provide a size, geometry, and polarity that is suitable to allow the compounds of the invention to fit within the binding regions of the biological target molecules. Therefore, in many embodiments, the Ar₂ radical, together with all its substituents, comprises from 2 to 18 carbon atoms, or from 3 to 15 carbon atoms, from 4 to 12, or from 5 and 12 carbon atoms.

In one embodiment of the invention Ar₂ is a substituted aryl or substituted heteroaryl radical having the formula: 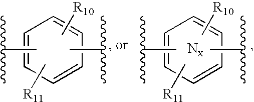
wherein x is 1 or 2, and R₁₀ and R₁₁ can be independently selected from hydrogen, inorganic radicals, or organic radicals, as those terms are defined elsewhere herein. In some embodiments, the inorganic radicals that can be employed as R₁₀ and R₁₁ substituent radicals are independently selected from hydroxyl, amino, or a halogen. In some embodiments at least one of R₁₀ and R₁₁ are organic substituents having from 1 to 6 carbon atoms, or from 1 to 4 carbon atoms. In some embodiments, R₁₀ and R₁₁ are independently selected from hydrogen, a halogen, hydroxyl, or an alkyl, cycloalkyl, alkoxy, or haloalkoxy radical comprising 1 to 4 carbon atoms.

In some embodiments, the Ar₂ radical has “para” bond connecting Ar₂ to the Ar₁ and the atom that links Ar₂ to the HAr radical, so as to have the formula: 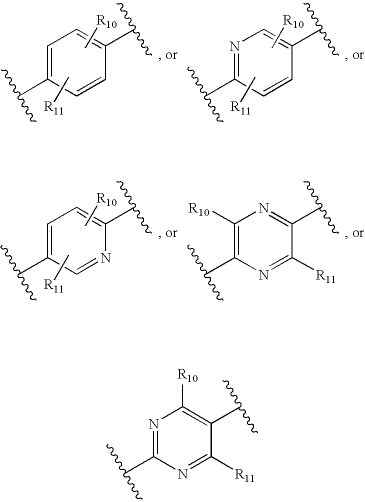
wherein R₁₀ and R₁₁ are defined as shown above.

In some embodiments, the compounds of claim 1 have an unsubstituted Ar₂ radical having the structure:

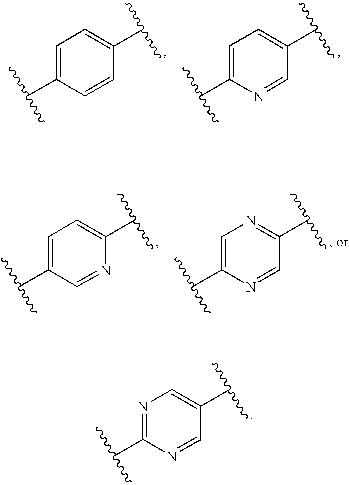

In additional aspects, the invention relates to compounds of Formulas (I) or (II) wherein Ar₂ has the structure: 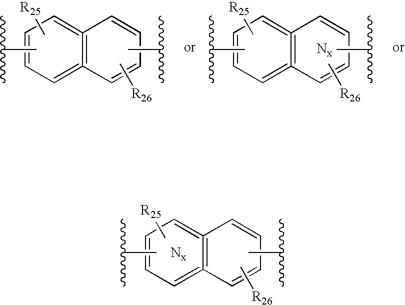
wherein x is 1 or 2, and R₂₅ and R₂₆ are independently selected from hydrogen or an alkyl, a substituted alkyl, an alkenyl, a substituted alkenyl, an alkynyl, a substituted alkynyl, a cycloalkyl, a substituted cycloalkyl, a heterocyclic, a substituted heterocyclic, an alkoxy, a substituted alkoxy, a hydroxyl, an acyl, an amino, a mono-substituted amino, a di-substituted amino, a carboxy, a carboalkoxy, an alkylcarboxamide, a substituted alkylcarboxamide, a dialkylcarboxamide, a substituted dialkylcarboxamide, a haloalkoxy. In some further aspects Ar₂ can have the structure:

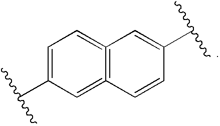

In additional embodiments, the compounds of the invention may comprise an Ar₂ radical having the structure:

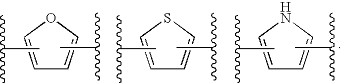

The HAr of the compounds of Formulas (I) and (II) comprises a five membered heterocyclic ring that comprises at least one carbon atom and at least one nitrogen atom, which may or may not have additional substituents bound thereto. The five membered heterocyclic HAr ring can also comprise oxygen or sulfur atoms, or carbonyl or thiocarbonyl, or thionyl radicals.

The HAr radicals that may be present in the compounds of Formulas (I) and (II) include but are not limited to five membered heterocycles having the formulas:

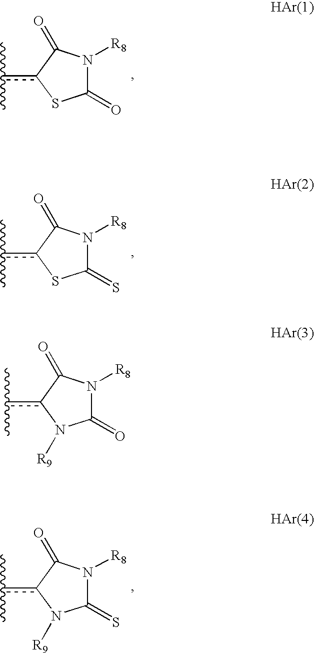

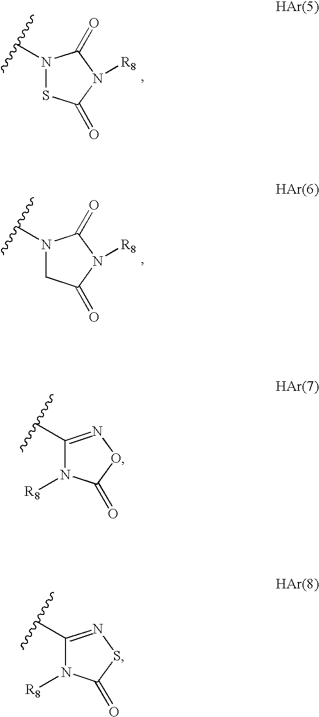

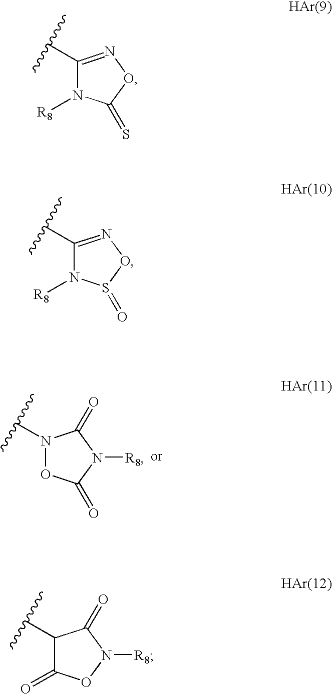

For the above HAr(x) heterocycles, R₈ and R₉ can be independently selected from the group consisting of hydrogen, or an organic radical having 1 to 10 carbon atoms. In some embodiments, R₈ and R₉ can be independently selected from hydrogen or a lower alkyl radical.

In many embodiments, R₈ and/or R₉ are hydrogen. When R₈ and/or R₉ are hydrogen, the HAr(x) heterocycles can be named as follows:
HAr(1)=1-substituted-thiazolidine-2,4-dione;
HAr(2)=1-substituted-2-thioxo-thiazolidin-4-one;
HAr(3)=1-substituted-imidazolidine-2,4-dione;
HAr(4)=1-substituted-2-thioxo-imidazolidin-4-one;
HAr(5)=2-substituted-[1,2,4]thiadiazolidine-3,5-dione;
HAr(6)=1-substituted-imidazolidine-2,4-dione;
HAr(7)=3-substituted-4H-[1,2,4]oxadiazol-5-one;
HAr(8)=3-substituted-4H-[1,2,4]thiadiazol-5-one;
HAr(9)=3-substituted-4H-[1,2,4]oxadiazole-5-thione;
HAr(10)=4-substituted-3H-[1,2,3,5]oxathiadiazole 2-oxide;
HAr(11)=2-substituted-[1,2,4]oxadiazolidine-3,5-dione;
HAr(12)=4-substituted-isoxazolidine-3,5-dione.

Some of the HAr(x) heterocyclic residues described above can exist in various tautomeric forms, as is known to those of ordinary skill in the art. It is to be understood that all such tautomers are within the scope of the invention.

In some embodiments of the invention, the compounds of the invention comprise only HAr(1), HAr(2), HAr(3), or HAr(4) radicals, wherein R₈ and R₉ are hydrogen, i.e.;

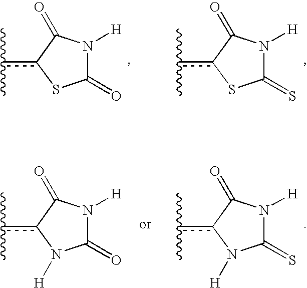

In some embodiments of the invention, the compounds of the invention comprise only HAr(1), HAr(2), wherein R₉ is hydrogen, i.e.;

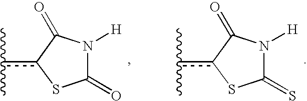

Some embodiments of the invention relate to compounds having a carbon atom bearing an R₃ radical substituent to link the Ar₂ radical and the HAr radical, as shown below: 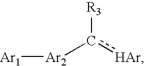
wherein ----- represents a bond present or absent, so that either a single bond or a double bond may exist between the linking carbon atom and HAr, as shown below;

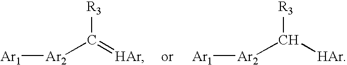

When ----- is present, both E and Z configurations of the double bond, or a mixture of both E and Z geometries of the double bond are within the scope of the invention. For example, the compounds of Formula (I) wherein ----- is present and HAr is Thiazolidine-2,4-dione include compounds of both the isomeric formulas shown below.

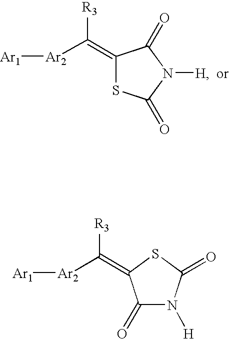

It is to be understood that for the purposes of this document, including the description and claims, if a chemical drawing shows only one of the two E or Z isomers as shown above, it should be presumed that either of the illustrated E or Z isomers, or a mixture of the two E and Z isomers is intended unless it is otherwise clear to the contrary from the context or claims. In experimental practice, especially as shown in the examples below, mixtures of the two E and Z isomers are sometimes obtained, although one isomer can substantially predominate over the other isomer in many actual experiments, depending upon experimental conditions. In the examples below, the chemical drawings illustrate the E or Z isomers that was experimentally observed to predominate in the particular example.

Overall, some embodiments of the invention relate to compounds having the structure: 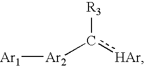
wherein:
a) Ar₁ has the structure: 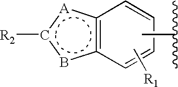
wherein
   i) R₁ is hydrogen, an inorganic radical, or an organic radical comprising 1 to 18 carbon atoms;
   ii) R₂ is selected from the group consisting of hydrogen, an inorganic radical, or a organic radical having 1 to 7 carbon atoms;
   iii) A and B are independently selected from the group consisting of —O—N—, —NR₄—, and —S—, wherein at least one of A or B is —N— and R₄ is hydrogen or an organic radical comprising 1 to 4 carbon atoms, and C is carbon;
b) Ar₂ comprises 2 to 18 carbon atoms and is an aryl, a substituted aryl, a heteroaryl or a substituted heteroaryl, wherein the heteroaryl and substituted heteroaryl have one to three ring heteroatoms selected from the group consisting of O, S, and N;
c) R₃ is hydrogen, halogen, hydroxy, or an organic radical comprising 1 to 4 carbon atoms.
d) [symbol] represents a bond present or absent; and
e) HAr has the formula: 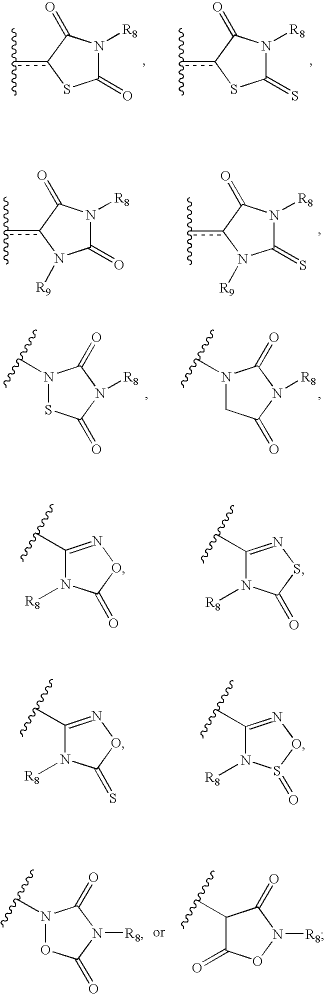
wherein R₈ and R₉ are independently selected from the group consisting of hydrogen, or an organic radical having 1 to 10 carbon atoms;
or a pharmaceutically acceptable salt thereof.

Further embodiments of the invention relate to compounds having the structure: 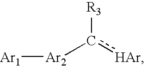
wherein:
a) Ar₁ has the structure: 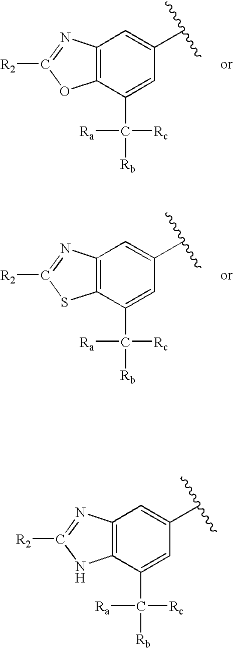
wherein
   i) Rₐ, R_{b}, and R_{c} are independently selected from hydrogen and alkyls, wherein two or three of the Rₐ, R_{b}, and R_{c} radicals can optionally together form cyclic, bicyclic, polycyclic rings, and with the proviso that no more than one of Rₐ, R_{b}, and R_{c} are hydrogen, and that Rₐ, R_{b}, and R_{c} together comprise between 3 and 11 carbon atoms;
   ii) R₂ is selected from the group consisting of hydrogen, amino, or a monosubstituted amino, disubstituted amino, alkoxy, or alkyl radical having 1 to 4 carbon atoms;
b) Ar₂ has the structure; 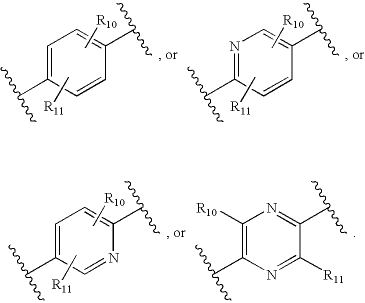
wherein the R₁₀ and R₁₁ substituent radicals are independently selected from hydrogen, hydroxyl, amino, halogen, or organic radicals comprising 1 to 4 carbon atoms independently selected from alkyl, alkoxy, haloalkyl, and haloalkoxy radicals;
c) R₃ is hydrogen, or an alkyl radical comprising 1 to 4 carbon atoms;
d) [symbol] represents a bond present or absent; and
e) HAr has the formula 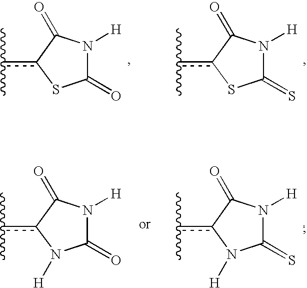
or a pharmaceutically acceptable salt thereof.

In yet further embodiments, the invention relates to compounds of the formula 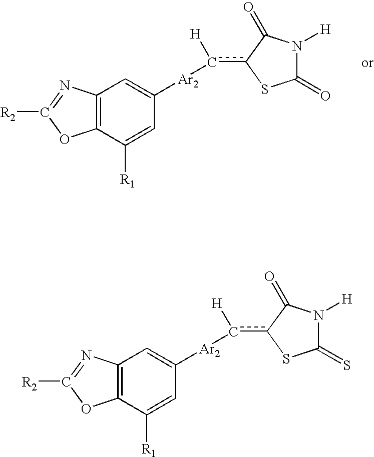
wherein:
(a) R₁ comprises 4 to 12 carbon atoms and is selected from the group consisting of an alky, a cycloalkyl, a heterocyclic, a heteroaryl, or an aryl;
(b) R₂ is selected from the group consisting of hydrogen, —SH, —NH₂, or an organic radical having 1 to 4 carbon atoms;
(c) Ar₂ has the formula 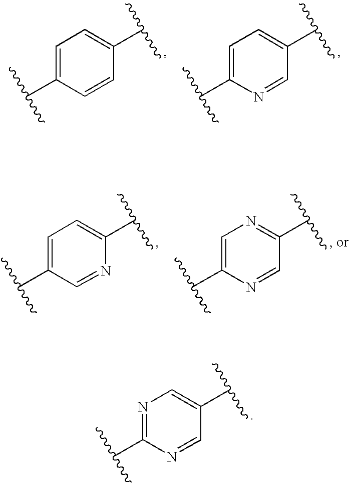
(d) [symbol] represents a bond present or absent;
or a pharmaceutically acceptable salt thereof.

In further embodiments related to the genus of compounds disclosed immediately above, R₁ has the formula 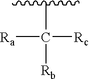
wherein Rₐ, R_{b}, and R_{c} together comprise from 3 to 12 carbon atoms and are independently selected from the group consisting of alkyl, cycloalkyl, or heterocyclic radical.

In further embodiments related to the genus of compounds disclosed immediately above, Rₐ, R_{b}, and R_{c} together form a cycloalkyl, or substituted cycloalkyl, or a heterocyclic, or substituted heterocyclic ring having from one to three heteroatoms selected from O, N, or S.

In further embodiment, R₁ has the formula

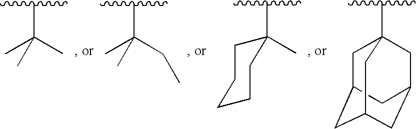

In additional embodiments similar to those disclosed above, the compounds of the invention can include compounds of Formula (II) wherein a heteroatom “U” links Ar₂ to the HAr radical.

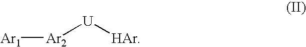

In the compounds of Formula (II), Ar₁, Ar₂ and HAr can be defined as in any of the embodiments described above, and U is a linking group selected from the group consisting of —NR₃—, —O—, —S—, —SO, and —SO₂—.

It is understood that when a chiral atom is present in a compound disclosed herein, both separated enantiomers, racemic mixtures and mixtures of enantiomeric excess are within the scope of the invention. As defined herein, racemic mixture is an equal ratio of each of the enantiomers, whereas an enantiomeric excess is when the percent of one enantiomer is greater than the other enantiomer, all percentages are within the scope of the invention. Furthermore, when more than one chiral atom is present in a compound then the enantiomers, racemic mixtures, mixtures of enantiomeric excess and diastereomic mixtures are within the scope of the invention.

The compounds disclosed herein can also include salts of the compounds, such as salts with cations, in order to form a pharmaceutically acceptable salt. Cations with which the compounds of the invention can form pharmaceutically acceptable salts include alkali metals, such as sodium or potassium; alkaline earth metals, such as calcium; and trivalent metals, such as aluminum. The only constraint with respect to the selection of the cation is that it should not unacceptably increase the toxicity. Also, one or more compounds disclosed herein can include salts formed by reaction of a nitrogen contained within the compound, such as an amine, aniline, substituted aniline, pyridyl and the like, with an acid, such as HCl, carboxylic acid and the like. Furthermore, all possible salt forms in relationship to the tautomers and a salt formed from the reaction between a nitrogen and acid are within the scope of the invention.

The acidity of some of the HAr heterocycles provides a ready method for preparing salts of the compounds of the invention, by reaction with an appropriate base, so as to generate a heterocyclic anion from the compound of the invention and a cation derived from the base employed. For example, the salts formed by such reactions can have the structure

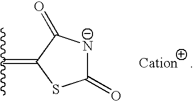

A wide variety of bases could be employed to produce such salts, including monovalent alkali metal hydroxides, divalent alkaline earth metal hydroxides, or bases comprising trivalent metal salts such as aluminum. Alternatively, organic bases such as primary, secondary, or tertiary amines can react with the acidic hydrogens of the compounds of the invention to form ammonium salts. The base and/or its associated cation are chosen so as to provide desirable solubility, toxicity, and/or bioavailability characteristics in the salt after formation of the desired salts. The identity of the base and/or the resulting cation will of course vary somewhat with the identity of the compound of the invention, and the nature of the pharmaceutical composition to be employed and its physical form as a solid or liquid, and the nature of any solvents and/or carriers employed.

Nevertheless, the United States Food and Drug Administration has published a list of pharmaceutically acceptable cations for pharmaceutically acceptable salts that includes aluminum, calcium, lithium, magnesium, potassium, sodium, and zinc cations, ammonium cations formed by the reactions of acidic compounds, with benzathine, chloroprocaine, choline, diethanolamine, ethylenediamine, meglumine, procaine, t-butylamine, and tris(hydroxymethyl)aminomethane (“Tris”). Such “pharmaceutically acceptable” salts are often employed and/or evaluated for use in the invention simply because of the likelihood of decreased FDA regulatory scrutiny. Example 25 provides an example of the synthesis of a particularly useful “Tris” salt of one of the compounds of the invention.

Also, one or more compounds disclosed herein can include zwitterionic salts formed by reaction of a nitrogen contained internally within the compound, such as an amine, aniline, substituted aniline, pyridyl and like residues with the acidic hydrogen of the HAr group.

This invention also encompasses pharmaceutical compositions containing prodrugs of the compounds of the invention as disclosed herein. The term “prodrug” means a drug precursor which, following administration, releases the drug (e.g., a compound of the present invention) in vivo via some chemical or physiological process. For example, a prodrug on being brought to the physiological pH or through enzyme action is converted to the desired drug form. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A. C. S. Symposium Series, and in Bioreversible Carners in Drug Design, ed. Edward B. Roche, American Pharmaceutical Association and Pergamon Press, 1987, the text of both of which treatises is hereby incorporated herein by reference, for their teachings regarding the structures, uses, properties, and preparations of prodrugs.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as .beta.-dimethylaminoethyl), carbamoyl-(C₁-C₂)alkyl, N,N-di(C₁-2)alkylcarbamoyl-(C₁-C₂)alkyl and piperidino-, pyrrolidino- or morpholino(C₂-C₃)alkyl.

Similarly, if a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, .alpha.-amino(C₁-C₄)alkanoyl, arylacyl and .alpha.-aminoacyl, or .alpha.-aminoacyl-.alpha.-aminoacyl, where each .alpha.-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention comprises an amine functional group, a prodrug can be formed by the replacement of a hydrogen atom in the amine group with a group such as R-carbonyl, RO-carbonyl, NRR′-carbonyl where R and R′ are each independently ((C₁-C₁₀)alkyl, (C₃-C₇)cycloalkyl, benzyl, or R-carbonyl is a natural .alpha.-aminoacyl or natural .alpha.-aminoacyl-natural .alpha.-aminoacyl, —C(OH)C(O)OY wherein (Y is H, (C₁-C₆)alkyl or benzyl), —C(OY₀)Y₁ wherein Y₀ is (C₁-C₄)alkyl and Y₁ is ((C₁-C₆)alkyl, carboxy(C₁-C₆)alkyl, amino(C₁-C₄)alkyl or mono-N— or di-N,N—(C₁-C₆)alkylaminoalkyl, —C(Y₂)Y₃ wherein Y₂ is H or methyl and Y₃ is mono-N— or di-N,N—(C₁ —C₆)alkylamino, morpholino, piperidin-1-yl or pyrrolidin-1-yl.

Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula 1. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, omithine and methionine sulfone. Prodrugs also include compounds wherein carbonates, carbamates, amides and alkyl esters which are covalently bonded to the compounds of formula I or II. The prodrugs themselves may be in the form of a pharmaceutically acceptable salt.

The present invention also provides, but is not limited to, the specific compounds set forth in the Examples set forth below, and a pharmaceutically acceptable salt thereof.

Makin2 the Compounds of the Invention

Various synthetic methods and/or strategies can be employed in the synthesis or production of compounds having Formulas (I) and (II) as described above. Several such synthetic methods and/or strategies will be disclosed hereinbelow.

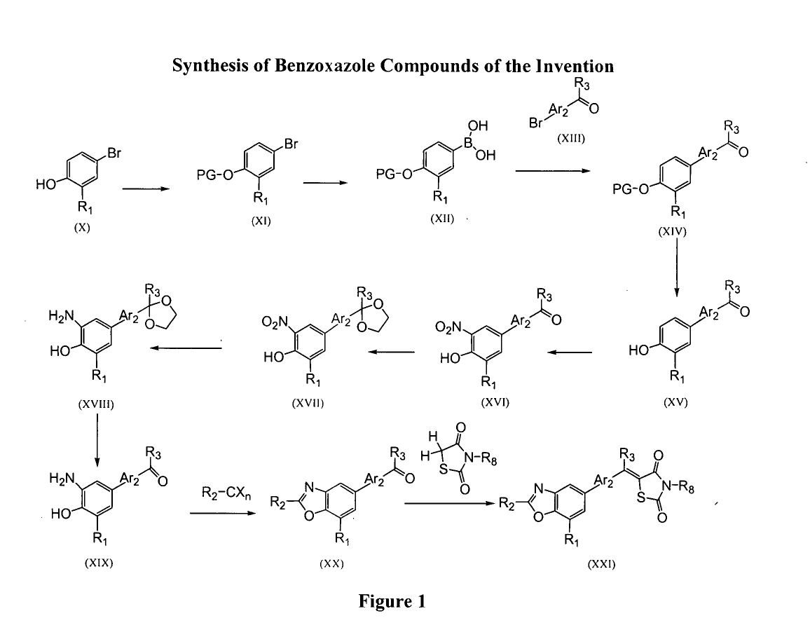

FIG. 1 illustrates a suitable synthetic pathway for synthesizing certain classes of benzoxazole compounds of Formula (I). FIG. 1 also generally illustrates certain useful synthetic strategies and reactions that can be modified to provide synthetic methods for benzothiazole and benzimidazole compounds of Formulas (I) and (II), as will be apparent to those of ordinary skill in the art, when read in light of their general knowledge, and the disclosures herein and in the prior art.

A desirable starting material for the synthesis of some isomers of the benzoxazole compounds of the invention are the halophenols, shown in the drawing below wherein Hal is Cl, Br, or I.

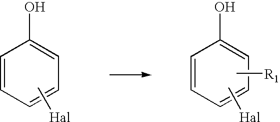

All possible isomers of these halophenols are commercially available from Aldrich Chemical Company of Milwaukee Wis., U.S.A. Such halophenols can be readily substituted with a variety of R₁ substitutents by a variety of methods that are well known to those of ordinary skill in the synthetic organic chemistry arts. The R₁-substituted-4-bromophenols (X) shown in FIG. 1 are particularly useful synthetic starting materials for the compounds of the present invention.

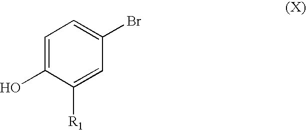

A number of desirable compounds of formula (X) are commercially available or can readily be synthesized by methods known in the literature. One method for synthesizing such compounds is recited in Example 1(i), which describes an acid catalyzed condensation reaction of 1-adamantol with 4-bromophenol, to provide 2-adaman-1-yl-4-bromophenol. Similar condensation reactions can be employed to provide other desired R₁ radicals, such as isopropyl, cyclohexyl, t-butyl, t-amyl, an substituted adamantyl radicals. Similar alkyl or substituted alkyl radicals can also be introduced by Friedel Crafts alkylations. Compounds of Formula (X) having acyl R radical substituents can be synthesized by Friedel Crafts acylation reactions of bromophenols. Compounds of Formula (X) having nitro R₁ radicals can be synthesized by nitration, and the resulting nitro-bromophenol reduced to provide 2-amino-4-bromophenol, which can then be alkylated or acylated on the amino group to provide compounds wherein R₁ is a monosubstituted or disubstituted amino radical, or an organoamide group.

The hydroxyl group of bromophenol (X) can be a precursor of the benzoxazole ring of Ar₁ radicals. In some synthetic methods of the invention, such as that shown in FIG. 1, it is desirable to protect the acidic hydroxyl group with a suitable protecting group PG, to provide the protected phenol (XI). Various suitable protecting groups are known to those of ordinary skill in art, one of which is the 4-t-butyldimethylsilanyloxy protecting group whose use is exemplified in Example 1 (h).

The protected bromophenol (XI) is a precursor of the Ar₁ radical that is suitable for coupling with an appropriate precursor for the Ar₂ radical that can be an aryl halide (including aryl iodides, bromides, or chlorides), aryl triflates or aryl diazonium tetrafluoroborate. As shown in Figure (I), in some embodiments of the invention aryl boronic acid or ester such as compound (XII) is coupled with a suitable precursor of Ar₂ (such as bromo compound (XIII)) in presence of a palladium catalyst, to provide a biaryl compound of Formula (XIV). This type of aryl coupling reaction is often generically termed a “Suzuki” coupling reaction, and such reactions are generally described respectively in Suzuki, Pure & Applied Chem., 66:213-222 (1994), Miyaura and Suzuki, Chem. Rev. 95:2457-2483 (1995), Watanabe, Miyaura and Suzuki, Synlett. 207-210 (1992), Littke and Fu, Angew. Chem. Int. Ed., 37:3387-3388 (1998), Indolese, Tetrahedron Letters, 38:3513-3516 (1997), Firooznia, et. al., Tetrahedron Letters 40:213-216 (1999), and Darses, et. al., Bull. Soc. Chim. Fr. 133:1095-1102 (1996); all of which are incorporated herein in their entirities by reference.

In some applications of these “Suzuki”coupling reactions to the present inventions, the protected bromophenol (XII) can be lithiated (for example with n-butyl lithium, as described in Example 1(g)) and then reacted with a borate ester to produce an aryl borate ester (XIIa) as shown below, wherein R₅₀ can be hydrogen, alkyl, or an alkylene group, so as to form an aryl borate ester heterocycle. The aryl borate esters can be used directly for coupling with a precursor of Ar₂, or can be hydrolyzed to provide an aryl boronic acid of Formula (XII) shown in FIG. 1, which is also suitable for Suzuki coupling.

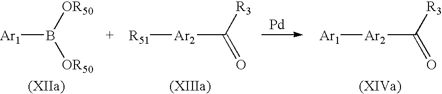

The coupling reactions to form biaryls comprising the Ar₁ and Ar₂ radicals are sometimes more advantageously conducted using certain boronic esters, such as where R₅₀ together with the boron form a pinacol borate ester (formation of pinacol esters: Ishiyama, T., et al., J. Org. Chem. 1995, 60, 7508-7510, Ishiyama, T., et al., Tetrahedron Letters 1997, 38, 3447-3450; coupling pinacol esters: Firooznia, F. et al., Tetrahedron Letters 1999, 40, 213-216, Manickam, G. et al., Synthesis 2000, 442-446; all four of which references are hereby incorporated herein by reference).

The aryl borate acid or ester precursor of Ar₁ can then be coupled with precursors of Ar₂, such as aryl compounds (XIIIa) shown above, wherein R₅₁ is a halide such as, iodo, bromo, or chloro, or a triflate or diazonium tetrafluoroborate. The aryl bromide compound (XIII) in FIG. 1 is an example of such an Ar₂ precursor compound. In view of the disclosure herein regarding the varieties of structures that are possible for the Ar₂ radical, a variety of substituted aromatic or heteroaromatic compounds are required as synthetic precursors of Ar₂, such as for example compound (XIIIa) above, and compound (XIII) in FIG. 1. Many such substituted precursor compounds are commercially available, or can be obtained by methods disclosed in the voluminous known prior art relating to methods for the synthesis of substituted organic and/or aromatic compounds, or are provided in the Examples attached herewith. A summary of the many synthetic methods and/or procedures that can be utilized for the synthesis of precursor compounds needed for the synthesis of a particular final product compound, or a suitable synthetic precursor thereof, and can be found, for example, in Smith, M. and March, J., Advanced Organic Chemistry, 5^{th} Edition, Weiley-Interscience (2001); or Larock, R. C., Comprehensive Organic Transformations, A Guide to Functional Group Preparations, Wiley, Inc. (1999), the disclosure of both of which references are hereby incorporated herein by reference, for their disclosures of the methods of synthetic organic chemistry. One of ordinary skill in the synthetic organic chemistry arts art could, by employing their general knowledge, in light of the disclosures of the prior art and the guidance provided herein, readily synthesize and obtain useful quantities of the synthetic precursors required for most or all of the Ar₂ radicals contemplated herein, without resort to exertion of undue or excessive experimentation.

The coupling reaction of (XIIa) with (XIIIa) is carried out in the presence of palladium catalyst complexes, as described in the references cited above and exemplified in Example 1(f) below. Those of skill in the art are aware that a number of variations on such “Suzuki” coupling procedures are known, and can be advantageously employed in the various embodiments of the present inventions. For example, it is known and understood by those of ordinary skill that the identity of the coupling groups can be “reversed” to achieve the same coupling product compound, as shown below by compounds: 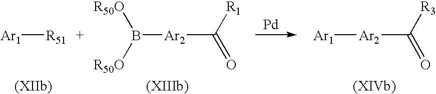
wherein R₅₀ and R₅₁ have the same meaning as described above. The conversion of compound (XI) in FIG. 1 to the biaryl carbonyl containing precursor compound (XIV) shown in FIG. 1 can be carried out by either variation of the Suzuki method as shown above.

The coupling of the Ar₁ and Ar₂ radicals can also be conducted by coupling an aryl zinc halide and an aryl halide or triflate. Alternately, the coupling reaction can also be executed using an aryl trialkyltin derivative and an aryl halide or triflate. These coupling methods are reviewed by Stanforth, Tetrahedron 54:263-303 (1998) and the content of those references is incorporated herein by reference, for the purpose of applying those synthetic methods to the synthesis of the compounds of the present invention. In general, the utilization of a specific coupling procedure to couple the Ar₁ and Ar₂ radicals is selected by consideration of several factors, including available precursors, chemoselectivity, regioselectivity and steric considerations.

Once the protected biaryl carbonyl compound (XIV) shown in FIG. 1 has been synthesized, by the coupling methods described above or any other known methods of organic chemistry as will be mentioned below, the protecting group for the phenolic hydroxyl is removed (as exemplified in example 1(e), to give the carbonyl containing biaryl (XV). The carbonyl containing biaryl (XV) can be nitrated by various know methods to form the nitrophenol compound (XVI) (see Example 1(d) for a procedure for nitration with nitronium tetrafluoroborate). Then the carbonyl group of the nitrophenol compound (XVI) is protected, for example by reaction with ethylene glycol to form a dioxolane compound (XVII) (see Example 1(c)), whose nitro group can be reduced to an amino group by various known catalytic or stoichiometric methods, to form the protected amino phenol compound (XVIII), which is then deprotected (see Example 1(b)) to form the ortho-aminophenol compound (XIX), which is the immediate precursor of the benzoxazole ring.

The ortho-aminophenol compound (XIX) can be condensed with a variety of reagents to close the benzoxazole ring and provide the R₂ substituent on the benzoxazole ring, to provide the benzoxazole compound (XX). A variety of such reagents, generically shown in FIG. 1 as “R₂—CXₙ” and methods for the condensation reactions will be further disclosed below.

The benzoxazole compound (XX) shown in FIG. 1 is an important synthetic intermediate, from which many of the final products compounds that comprise HAr heterocycles are derived. A variety of methods for attaching suitable HAr heterocycles to compound (XX) will be described below. FIG. 1 illustrates one class of synthetic reactions for attaching an HAr heterocycle, namely the “Knoevenagel” type condensation of the carbonyl carbon of compound (XX) with a heterocyclic compound having reactive hydrogen atoms attached to a methylene ring carbon atom, to produce compound (XXI) shown in FIG. 1, which represents a particular class of valuable thiazolidine-2,4-dione compounds.

“Knoevenagel” type condensation reactions have been described by Tietze and Beifuss in Comprehensive Organic Synthesis (Pergamon Press), 2:341-394, (1991), which is hereby incorporated herein in its entirety by reference. Such condensations can be employed to condense carbonyl containing precursor compounds such as (XX) with precursor heterocycles such as substituted or unsubstituted heterocyclic compounds such as thiazolidine-2,4-diones (to produce HAr(1)); 2-thioxo-thiazolidin-4-ones (sometimes referred to as “rhodanines”) to produce HAr(2); imidazolidine-2,4-diones to produce HAr(3); and 2-thioxo-imidazolidin-4-ones to produce HAr(4) radicals, as illustrated below, wherein R₈ and R₉ are hydrogen or another organic radical as defined elsewhere herein.

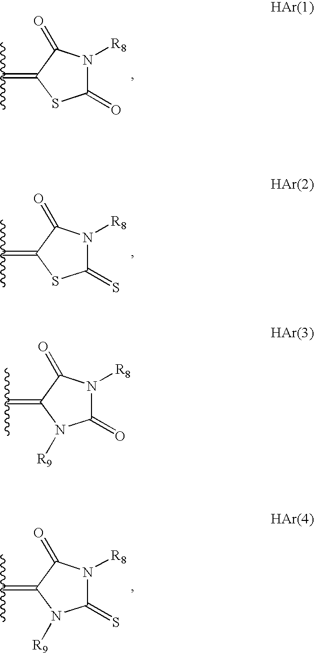

The Knoevenagel condensation reactions between carbonyl compounds such as (XX) and the heterocycles shown above are often conducted by refluxing in an appropriate solvent (such as toluene) in the presence of a catalytic amount of a suitable base, such as an alkyl amine, as is detailed in the Examples herein. Alkyl substituents for the nitrogen atoms of the heterocycles can be introduced by condensation reactions with known alkylating agents, such as alkyl halides, alkyl sulfonates, etc.

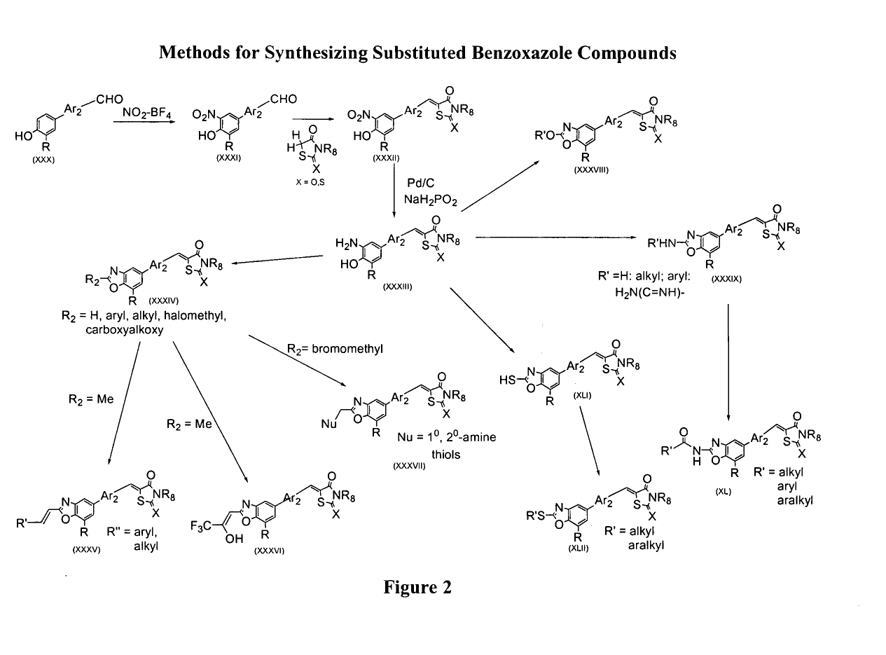

Alternative synthetic approaches for producing the benzoxazole compounds of the invention can also be employed. One such alternative approach is shown in FIG. 2, which also illustrates a variety of reagents that can be employed to form the benzoxazole ring and a variety of R₂ substituents for the benzoxazole ring. The biaryl phenol (XXX) shown in FIG. 2 is similar to previously mentioned intermediate (XV) wherein R₃ is hydrogen (for non-limiting purposes of illustration only). Compound (XXX) can be produced via aryl coupling reactions as disclosed above, or by other well-known methods of synthetic organic chemistry, such as Vilsmeier-Haack formylation of a corresponding biaryl compound.

The biaryl phenol (XXX) can be selectively nitrated ortho to the hydroxyl group to yield nitrophenol (XXXI), which can be condensed with a heterocyle of types HAr(1), HAr(2), HAr(3), or HAr(4), in a Knoevenagel type reaction to produce compounds of Formula (XXXII), which already comprise coupled Ar₁, Ar₂, and HAr radicals, but lack the benzoxazole radical. The nitro group can be selectively reduced in the presence of HAr heterocycles by catalytic hydrogenation using a Pd/carbon/sodiumphosphate catalyst (see K. Arakawa et al.: Chem. Pharm. Bull. 45 (1997) 1984) to produce a very flexible aminophenol intermediate (XXXIII).

Aminophenol intermediate (XXXIII) shown in FIG. 2 can be condensed with a variety of reagents to form the benzoxazole ring and provide final benzoxazole compounds with a wide variety of R₂ radicals. For example, aminophenol (XXXIII) can be converted to benzoxazoles of Formula (XXXIV) wherein R₂ can be hydrogen, an alkyl, an aryl, a haloalkyl, or a carboalkoxy group, by methods disclosed by Arakawa et al., by J. H. Musser et al., J. Med. Chem. 28 (1985) 1255, and/or by the methods cited in the Examples 1, 2, and 3 disclosed herein. When R₂ is a methyl group, the methyl group can be chemically reactive, and further elaborated to provide olefinic R₂ radicals, such as those of Formulas (XXXV) (see I. N. Houpis et al.: J. Org. Chem. 58 (1993) 3176) and (XXXVI) (see V. Dryanska et al.: Synthesis 37, (1976), and M. Kawase et al.: Heterocycles 48 (1998) 2103). When R₂ is bromomethyl, the bromide can be displaced by various nucleophiles, such as primary or secondary amines, or thiols, to provide compounds of Formula (XXXVII) (see Arakawa et al).

Aminophenol (XXXIII) can also be condensed with orthoesters to provide compounds of formula (XXXVIII), see W. Kantlehner et al.: Liebig's Ann. Chem. 507-529(1982). Aminophenol (XXXIII) can also be condensed with cyanogen bromide to yield compounds of Formula (XXXIX) wherein R′=hydrogen, see Example 14 and Katsura et al., followed by an optional further conversion of compound (XXXIX) to compound (XL). Compounds of Formula (XXXIX) wherein R′ is alkyl, aryl, or guanidino can be prepared by the methods disclosed by Y. Ito et al.: J. Organomet. Chem. 131, 121-131 (1977); E.-S. A. Ibrahim et al.: J. Heterocycl. Chem. 19, 761 (1982); and Acheson et al.: J. Chem. Soc. 4727 (1956).

Aminophenol (XXXIII) can also be condensed with KSCSEt to produce thiol compound (XLI), by reactions analogous to those disclosed by F. Haviv et al.: J. Med. Chem. 31, 1719 (1988), and E. S. Lazer et al.: J. Med. Chem. 37, 913 (1994). Thiol compound (XLI) can be further elaborated to provide the thioether compounds of Formula (XLII), by methods similar to those disclosed by R. W. DeSimone et al.: Bioorg. Med. Chem. Lett. 10, 2723 (2000).

Those of ordinary skill in the art will appreciate that other compounds within the scope of the inventions having structures related to those whose synthesis is described above, such as compounds with differing substitutent radicals on Ar₁, Ar₂, and HAr, can ordinarily be readily synthesized by varying the structure of the Ar₁ and Ar₂ starting materials, and/or using variations of the synthetic reactions disclosed herein. For example, Example 9 documents a synthetic strategy involving a “reverse” Suzuki coupling strategy as shown below.

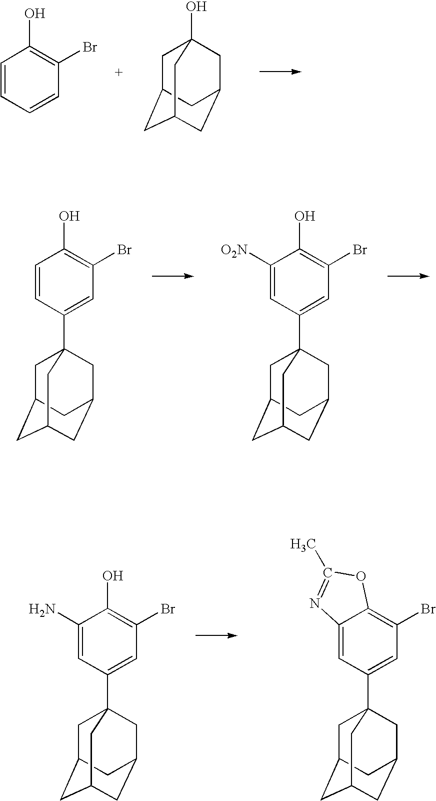

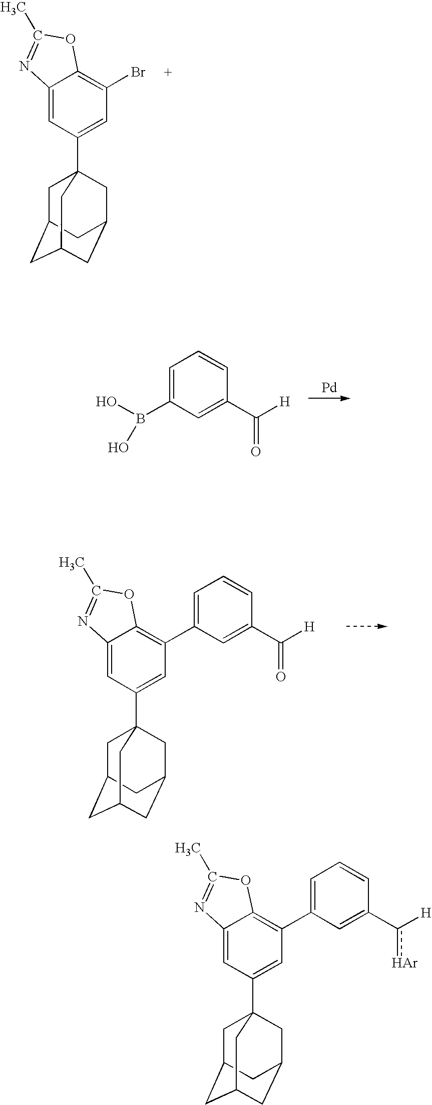

Many similar modifications of the overall synthetic strategies generally described herein for the synthesis of benzoxazole compounds of Formula (I), and the synthesis of the necessary precursor aromatic compounds to implement those strategies are within the level of ordinary skill in the synthetic organic chemistry arts. For example, 5-brominated benzoxazole compounds having the structure 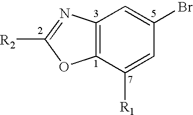
are precursors of the Ar₁ radicals of the compounds of the invention that can be obtained by employing “reverse” Suzuki couplings.

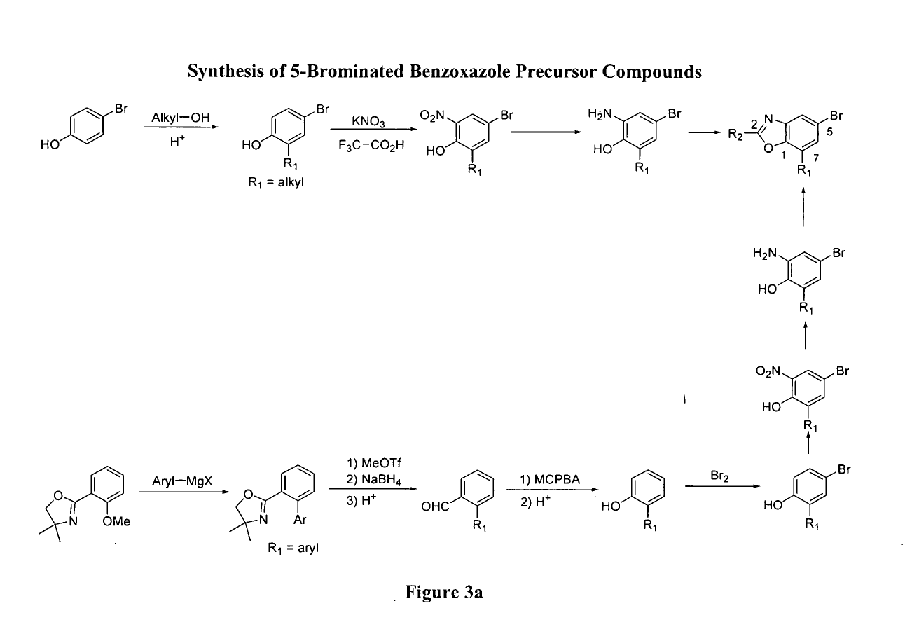

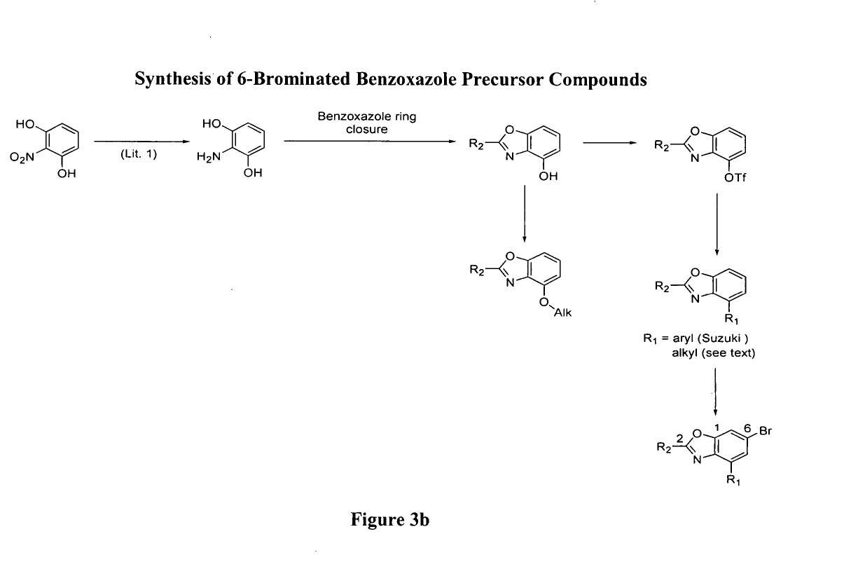

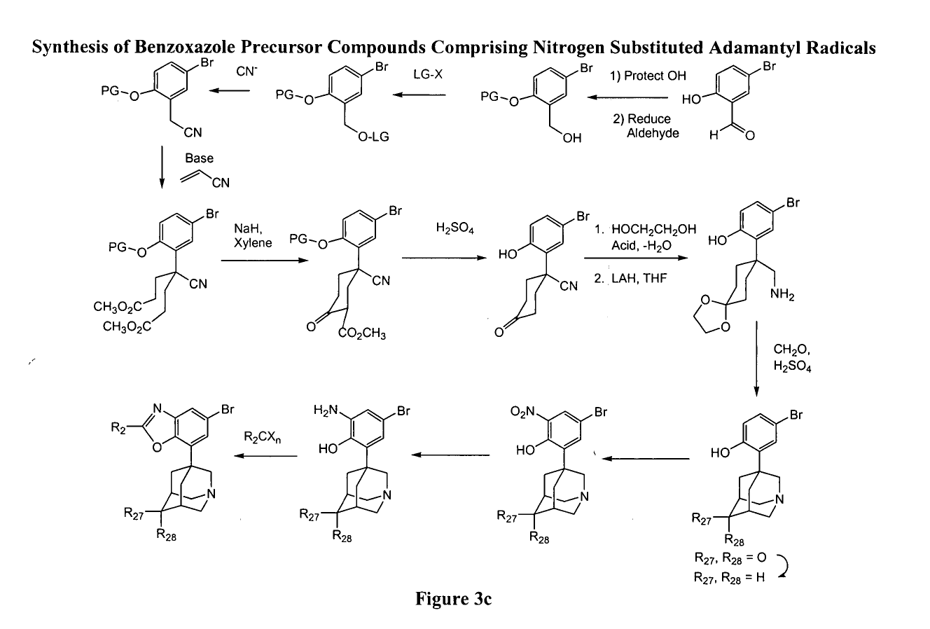

Two methods for synthesizing desirable precursors of the Ar₁ radicals, such as 5-brominated benzoxazole compounds, are shown in FIG. 3a. Para-bromophenol can be ring alkylated, nitrated, and the nitro group reduced to form an orthoaminophenol compound, which can be reacted with a variety of reagents as described hereinabove to close the benzoxazole ring and form the desired 5-bromobenzoxazole compounds with 7-alkyl substituents. Similar 7-aryl-5-bromobenzoxazoles can be prepared as shown in FIG. 3a, by using aryl substituted 2-oxazoline compounds to prepare 2-arylphenols, as described by Gant et. al., Tetrahedron, 50, 2297-2360 (1994), followed by subsequent bromination, nitration, reduction, and benzoxazole ring closure reactions analogous to those already described.

Alternatively, brominated precursors of Ar₁ having the positions of the oxygen and nitrogen atoms of the benzoxazole ring interchanged, so as to give 6-brominated benzoxazole precursor compounds having the structures shown below, can be prepared by the reactions shown in FIG. 3b.

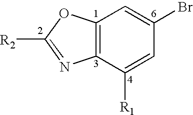

2-Nitroresourcinol (see FIG. 3b) is available from Aldrich Chemical Company of Milwaukee Wis., and can be reduced as taught by W. S. Saari et el.: J. Med. Chem. 35, 3792 (1992), to produce 2-aminoresourcinol, which can then be reacted by a variety of methods (including the method of J. H. Musser at al.: J. Med. Chem. 30, 62 (1987)) to produce a 4-hydroxy-benzoxazole. The hydroxyl group of the 4-hydroxy-benzoxazole can be reacted with triflating agents to yield a triflate suitable for Suzuki coupling to produce a 4-aryl-benzoxazole that can then be brominated (see Desai et al.: J. Chem. Soc., 321, (1938)). Equivalent brominated 4-alkyl-benzoxazole compounds can be obtained from the triflate by analogy to the method of G. Zou et al., as described in: Tetrahedron Lett. 42, 7213, (2001). Lastly, the previously mentioned 4-hydroxy-benzoxazole can be o-alkylated according to the method of D. T. Plummer et al.: J. Organomet. Chem. 260, 347 (1984), to produce benzoxazole Ar₁ precursor compounds having alkoxy R₁ substitutents.

Some compounds of the invention described comprise Ar₁ radicals having R₁ substitutents including certain “azaadamantyl” derivatives having the structures shown below:

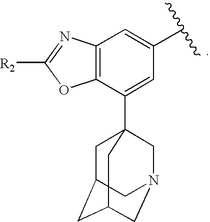

Examples of methods for synthesizing suitable precursors of such compounds are shown in FIG. 3c. 5-bromo-salicaldehyde (5-bromo-2-hydroxybenzaldehyde) is commercially available from Aldrich Chemical Co. of Milwaukee Wis., and provides a starting material for the synthesis of many desirable Ar₁ precursors comprising variously substituted benzoxazole and azaadamantyl radicals. The phenolic hydroxyl group of 5-bromo-salicaldehyde is protected with a suitable protecting group, then the aldehyde reduced by various well known methods to give a benzyl alcohol, whose benzylic hydroxyl can be derivatized with a suitable leaving group (such as tosylate or triflate) and displaced by cyanide to give a benzylic cyanide compound.

The benzylic cyanide can be treated with 2 equivalents of a cyanoacrylate, which may optionally contain various organic or inorganic substitutents on the acrylic double bond, to yield a dicarboxylic acid ester that can be cyclized in the presence of base, then decarboxylated and deprotected in the presence of acid, to yield cyano substituted benzylic cyclohexanone compounds.

The carbonyl group of the cyano substituted benzylic cyclohexanone compound shown in FIG. 3c can be directly reduced to the corresponding methylene derivative under Wolff Kishner conditions (reaction not shown in FIG. 3c), or the ketone group can be protected as an ethylene glycol ketal, followed by reduction of the cyano group to an amine with lithium aluminum hydride. The ketal of the amine compound is hydrolyzed in the presence of aqueous formaldehyde to close the azaadamantyl ring. If the ketone group of the azaadamantyl group is still present, it can be optionally reduced to a methylene group under Wolff Kishner conditions, then the resulting phenol selectively nitrated via several known procedures ortho to the phenolic hydroxyl group, and the resulting nitro compound selectively reduced to an ortho-aminophenol, which can be condensed with various reagents described elsewhere herein to close the benzoxazole ring and provide a bromo-benzoxazole compound that is a suitable precursor for Ar1 of the desired final compounds of the invention. Starting with appropriate starting thiophenols or anilines, similar benzothiazole or benzimidazole precursor compounds can be readily prepared by those of ordinary skill in organic synthetic chemistry arts.

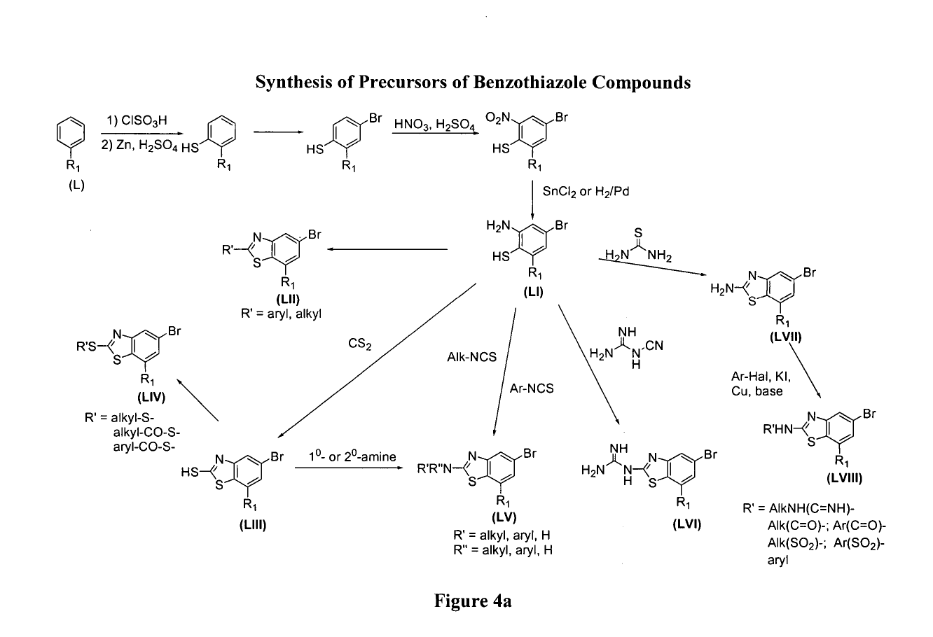

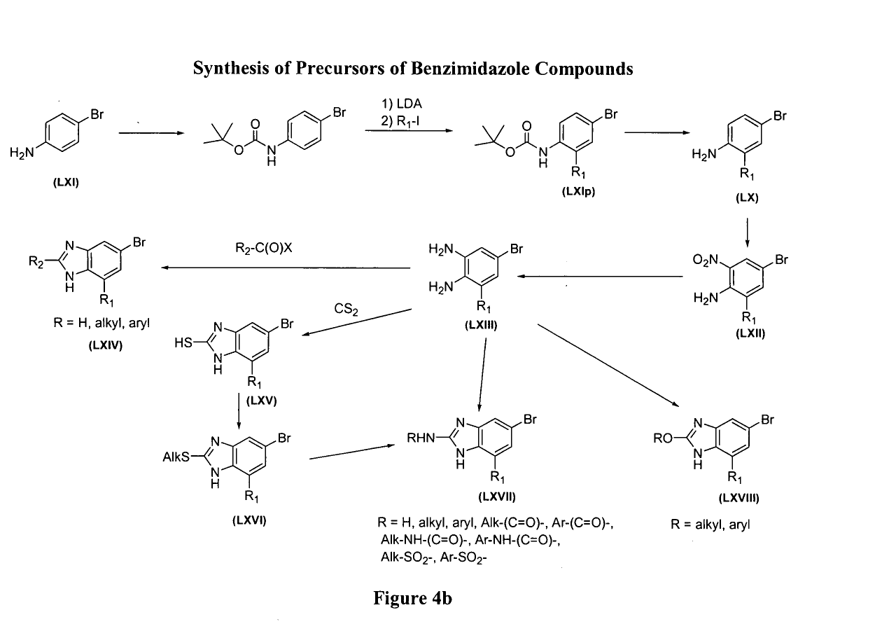

Via modification of the procedures described above, the synthesis of precursors of the benzimizole and benzothiazole compounds of the invention can be readily achieved by the synthesis of appropriate brominated benzothiazole and benzimidazole precursors for Ar₁. FIG. 4a illustrates exemplary synthetic strategies for producing brominated benzothiazole compounds that can be used as synthetic precursors for the Ar₁ radical. FIG. 4a illustrates a reaction sequence in which a compound (L) having a benzene ring substituted with an activating R₁ substituent (such as hydroxyl, alkoxy, alkyl, amino, protected amino, etc) can be transformed, via a sequence of sulfonation, reduction, halogenation, nitration, and reduction, (for analogous chemical reactions in other contexts, see Hansch et al.: J. Am. Chem. Soc. 70, 1561 (1948); U.S. Pat. No. 3,461,168, (1966); M. H. Elmagdi et al.: Phosphorus, Sulfur, Silicon, Relat. Elem. 82, 195 (1993); and L. Racane et al.: Heterocycles 55, 2085 (2001)) to produce a 6-substituted-2-Amino-4-bromo-benzenethiol intermediate (LI).

Ortho aminobenzenethiols of structure (LI) can be condensed with various reagents, in analogy to known synthesis of prior art aminobenzenethiols, to produce a wide variety of substituted brominated benzothiazole compounds as shown in FIG. 4a. Benzothiazoles having alkyl or aromatic R₂ radicals, shown as compound (LII), can be synthesized by methods analogous to those disclosed by Racane et al; C. A. Mathis: Bioorg. Med. Chem. Lett. 12, 295 (2002); and Mourtas et al., Tetrahedron Lett. 42, 2201 (2001). Compounds (LIII), wherein R₂ is —SH, can be produced by condensation with carbon disulfide, in analogy to R. D. Schoenwald et al.: J. Med. Chem. 27, 810 (1984). Compound (LIII) can be sulfur alkylated or acylated in analogy to the reactions disclosed by D. J. Brown et al.: Aust. J. Chem. 32, 2713 (1979); P. R. Blakemore et al: Syn. Lett. 26 (1998); and F. Roulleau et al.: Tetrahedron Lett. 24, 719 (1983). The thiol group of Compound (LIII) can also be displaced by primary or secondary amines, to produce compound (LV), in analogy to J. D'Amico: J. Org. Chem. 26, 3436 (1961), or can alternatively be produced by condensations with organic thiocyanates in analogy to E. E. Gilbert: J. Heterocycle. Chem. 6, 483 (1969), and J. Garin et al.: J. Heterocycl. Chem. 28, 359 (1991).

Guanidino compounds such as (LVI) can be produced by condensations of (LI) analogous to those of S. P. Sing et al.: Indian J. Chem., Sect. B 22, 370 (1983). Benzothiazole compounds having an amino R₂ radical such as (LVII) can be obtained via reactions disclosed in U.S. Pat. No. 2,575,614, (1950); and the resulting amino radical further substituted to give compounds of Formula (LVIII) by reactions analogous to those disclosed by Z.-G. Li et al.: J. Chem. Soc., Synop. 11, 470 (2001); T. Kiatagawa et al.: Chem. Pharm. Bull. 49, 335 (2001); J. S. Yadav et al.: Tetrahedron Lett. 39, 3259 (1998); R. M. Scarborough et al.: Bioorg. Med. Chem. Lett. 11, 1805 (2001); and M. A. El-Sherbeny: Arzneim. Forsch. 50, 848 (2000). The references listed above provide relevant examples and experimental procedures for analogs of the reactions illustrated in FIG. 3, and are hereby incorporated herein by reference for their teachings relating to such reactions, reagents, and experimental procedures needed to produce the benzothiazole compounds disclosed in FIG. 4a.

Related reactions can be employed to synthesize precursors of the benzimidazole compounds of the invention as is exemplified in FIG. 4b. One suitable starting material is the bromoaniline compound (LX) shown in FIG. 4b (and its geometrical isomers). Many such starting compounds are commercially available, or available via prior methods. Nevertheless, some compounds of Formula (LX) that are desirable for synthesizing precursors of Ar₁ that comprise benzimidazole rings are not always readily commercially available. Therefore, the invention provides a method for the synthesis of such compounds, via the reaction sequence illustrated in FIG. 4b, starting from bromoanilines such as compound (LXI), all possible isomers of which are available from Aldrich Chemical Company of Milwaukee Wis. The use of t-BOC protecting groups for anilines such as (LXI) is described by T. W. Greene and P. G. M. Wuts in Protective Groups in Organic Synthesis, 2nd Ed, J. Wiley & Sons, Inc, 327 (1991). The t-BOC protected bromo-aniline undergoes a directed lithiation reaction, and subsequent reaction with organic iodide compounds (see for example A. Cervantes et al., Can. J. Chem. 73, 336 (1995); and S. Caron et al.: J. Org. Chem. 63, 2054 (1998)) that can be carried out in the presence of the bromo substituent on the aromatic ring. The protected aromatic compound (LXIp) is then deprotected to yield the desired substituted bromoaniline (LX).

Bromoaniline (LX) can be directly Suzuki coupled with a desired precursor of Ar₂, and then further elaborated to introduce the benzimidazole ring (not shown), or alternatively can be elaborated to introduce the imidazole ring at the bromoaniline stage, as shown in FIG. 4b. Bromoaniline (LX) can be nitrated to give nitro compound (LXII), then the nitro group reduced (in analogy to the procedure of S. Grivas et al.: Acta Chem. Scand. 47, 521 (1993)) to produce a very flexible 3-substituted-5-Bromo-benzene-1,2-diamine intermediate (LXIII), which can be condensed with a variety of reagents to form desired benzimidazole rings.

Compound (LXIII) can be condensed with carboxylic acid derivatives to produce compounds of Formula (LXIV), wherein R₂ is hydrogen, an alkyl, or an aryl, in analogy to the reactions disclosed by M. L. Lopez-Rodriguez et al., J. Med. Chem. 42, 5020 (1999); J. A. Robl et al., J. Med. Chem. 44, 851 (2001); and K. V. Reddy et al., Indian J. Chem. Sect. B 23, 866 (1984). Compound (LXIII) can also be condensed with carbon disulfide to produce thiol compound (LXV), in analogy to the reactions described by G. D. Gupta et al., Indian J. Chem. Sect. B 19, 1035 (1980). Thiol compound (LXV) can be alkylated to provide thioether compound (LXVI) by reactions analogous to those disclosed by J. C. Hazelton et al. in Tetrahedron 51, 10771 (1995). The thioether R₂ group of thioether compound (LXVI) can be replaced by the variously substituted amino groups of compound (LXVII), in analogy to the disclosures. of S. H. Reich et al., J. Med. Chem. 35, 847 (1992); C. P. Kordik et al., Bioorg. Med. Chem. Lett. 11, 2287 (2001); C. W. Phoon et al., Bioorg. Med. Chem. Lett. 11, 1647 (2001); Z. Ejmocki et al., Pol. J. Chem. 59, 1279 (1985); and Hultquist et al., J. Am. Chem. Soc. 73, 2558 (1951).

Finally, compound (LXIII) can be reacted to provide the alkoxy substituted benzimidazoles of compound (LXVIII) by analogy to reactions described by Sandmeyer, Chem. Ber. 19, 2654 (1886); K. Kubo et al., J. Med. Chem. 36, 2182 (1993); and R. L. Webb et al., J. Heterocycl. Chem. 24, 275 (1987).

By employing various combinations and permutations of the synthetic reactions described above, it is possible to synthesize a genus of structurally related synthetic intermediates for the benzoxazole, benzothiazole, and benzimidazole compounds of the invention that all comprise carbonyl radicals, having Formula (LXX), whose structure is shown below: 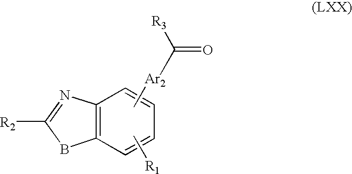
wherein B can be —O—, —S—, or —NR₄, and wherein R₁, R₂, R₃, R₄ and Ar₂ are defined as described hereinabove.

Compounds of Formula (LXX) can be readily synthetically elaborated to attach any of the HAr(1) to HAr(12) heterocycles disclosed above. Compounds of Formula (LXX) can for example, be condensed with precursors of heterocycles HAr(1), HAr(2), HAr(3), and HAr(4) under Knoevenagel conditions, to give heterocycles having the structures and Formulas shown below:

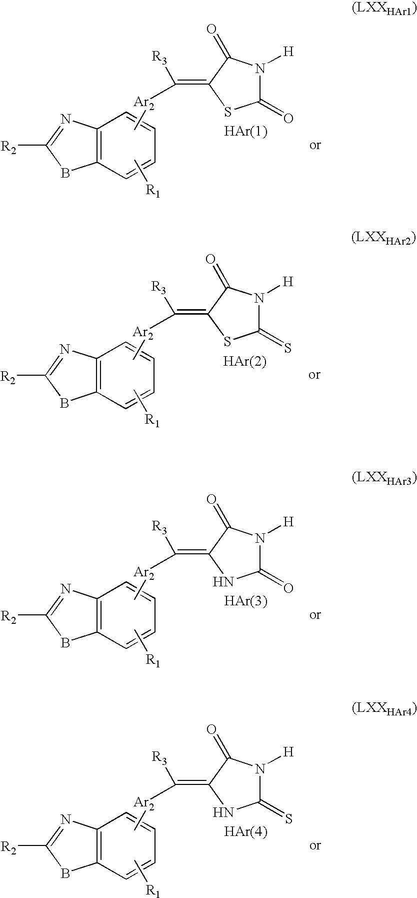

Moreover, when R₃ is hydrogen in compound (LXX), an aldehyde compound of the following structure will be formed, whose structure will be abbreviated for illustration purposes as follows:

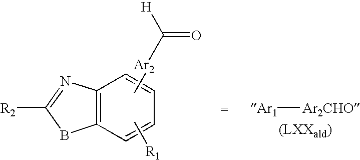

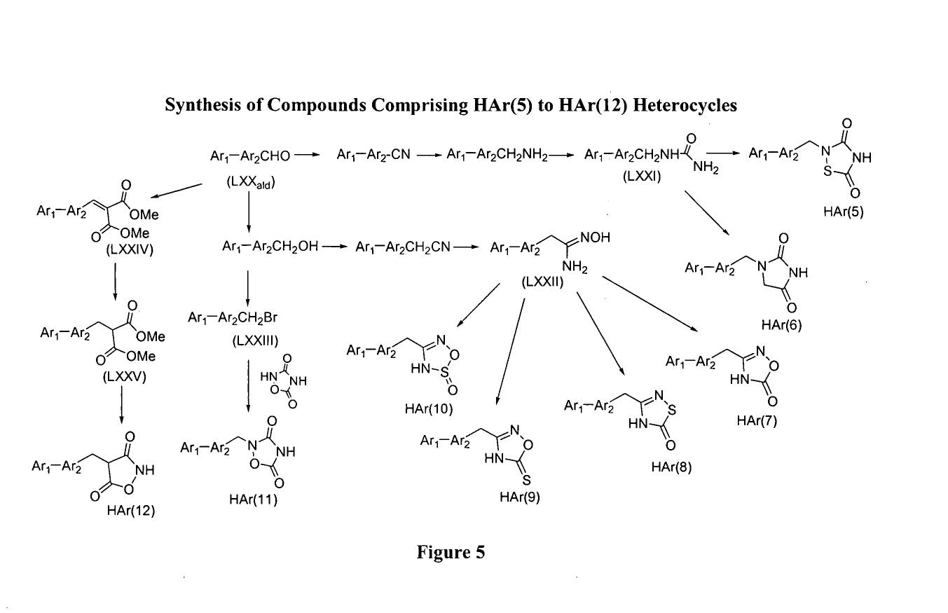

As shown in FIG. 5, “Ar₁-Ar₂CHO” compounds (having Formula (LXX_{ald}) can be alternatively elaborated to attach heterocycles of Formulas HAr(5) to HAr(12). Aldehydes of genus (LXX_{ald}) can be reacted with hydroxylamine and dehydrated to form an aryl cyanide compound, which can be reduced and/or hydrogenated to give a benzyl amine, which can be converted to the benzyl guanidine compound (LXXI). Benzyl guanidine compound (LXXI) can be reacted with chlorocarbonylsulfenyl chloride to give a compound of the invention comprising the HAr(5) (i.e. thiadiazolidinedione) heterocycle [see Malamas, M. et al., J. Med. Chem. 43, 995-1010 (2000)].

, or reacted with chloroacetic acid to give a compound comprising the HAr(6) (i.e. imidazolidinedione) heterocycle [see Zaidi, S. M. M et al, Pharmazie, 35(12), 755-756 (1980)].

As also shown in FIG. 5, aldehydes of genus (LXX_{ald}) can be reduced or hydrogenated by various known methods to form a benzyl alcohol, whose hydroxyl group can be substituted with a cyano group, which can then be reacted with hydroxylamine to form the N-Hydroxy-acetamidine compound (LXXII), which can then be further reacted to form compounds of the invention comprising heterocycles HAr(7), HAr(8), HAr(9), and HAr(10). See Ellingboe J. et al., J. Med. Chem. 36, 2485-2493 (1993); and Kohara Y. et al., J. Med. Chem. 39, 5228-5235 (1996) for analogous reactions, reagents, and reaction conditions. Moreover, the benzyl alcohols can be readily converted to benzyl bromides (LXXIII), which can be directly condensed with [1,2,4]oxadiazolidine-3,5-dione heterocycles of Formula HAr(11), to prepare the corresponding compounds of the invention, using procedures analogous to those reported by Cantello, B. et al; Synlett., 263-264 (1997).

Also, aldehydes of genus (LXX_{ald}) can be condensed with malonic acid diesters to form the benzylidene malonates of Formula (LXXIV) shown in FIG. 5, whose double bond can be reduced to form benzyl malonates (LXXV), which can then be cyclized in the presence of hydroxylamine to form benzylic compounds of the invention having HAr(12) (i.e. isoxazolidine-3,5-dione) heterocycles bonded thereto (see J. Med. Chem. 41, 1927-1933 (1998)).

Lastly, in FIG. 5, all the reactions attach the HAr(5)-HAr(12) heterocycles to the aldehyde group of an “Ar₁-Ar₂CHO” precursor compound. The same reaction sequences to attach five membered heterocycles can also be carried out on Ar₂ precursor compounds having the structures 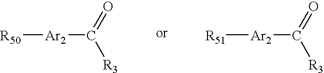
wherein R₃, R₅₀ and R₅₁ are as defined elsewhere herein, and then subjecting the resulting product compounds to coupling reactions to introduce the Ar₁ radical.

In view of the disclosures above, the inventions herein relate, in some embodiments, to a method for the synthesis of a benzoxazole, benzothiazole, or benzimidazole compound of the structure 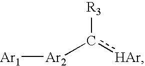
wherein:
a. Ar₁ has the structure: 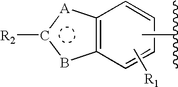
wherein
   i) R₁ is hydrogen, an inorganic radical, or an organic radical comprising 1 to 18 carbon atoms;
   ii) R₂ is hydrogen, halogen, —SH, —NH₂, or a organic radical having 1 to 7 carbon atoms;
   iii) A and B are independently selected from the group consisting of —O—, —S—, —N—, —NR₄—, and, wherein at least one of A or B is —N— and R₄ is hydrogen or an organic radical comprising 1 to 4 carbon atoms, and C is carbon;
b) Ar₂ comprises 2 to 18 carbon atoms and is an aryl, a substituted aryl, a heteroaryl or a substituted heteroaryl, wherein the heteroaryl and substituted heteroaryl have one to three ring heteroatoms selected from the group consisting of O, S, and N;
c) R₃ is hydrogen, halogen, hydroxy, or an organic radical comprising 1 to 4 carbon atoms.
d) [symbol] represents a bond present or absent;
e) HAr has the formula: 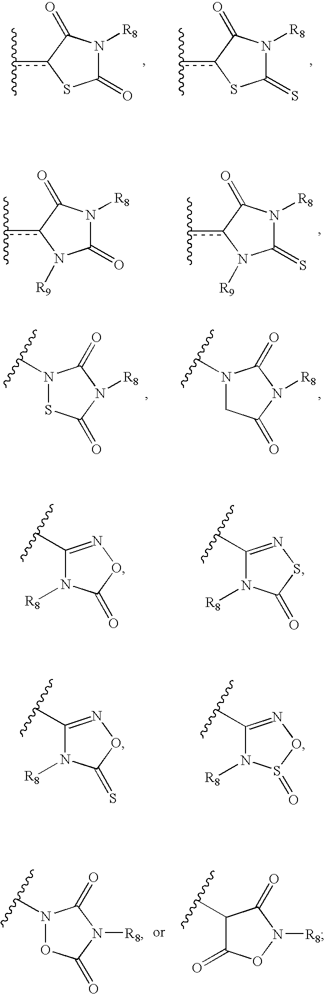
wherein R₈ and R₉ are independently selected from the group consisting of hydrogen, or an organic radical having 1 to 10 carbon atoms;
or a pharmaceutically acceptable salt thereof,
(e) the method comprising the steps of:
   1) coupling a first aryl compound with a second aryl compound to give a biaryl compound;
      wherein the first aryl compound has the structure: 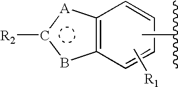
      and wherein the second aryl compound comprises a carbonyl group and has the structure: 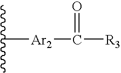
      and wherein the biaryl compound has the structure: 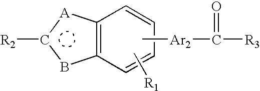
   and
   2) further reacting the biaryl compound so as to bond thereto the HAr radical, to form the benzoxazole, benzothiazole, or benzimidazole compound.

In further embodiments of the above method of synthesis, ----- represents a bond present, and HAr has the formula:

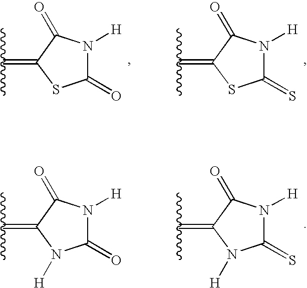

As described above, reaction of the biaryl carbonyl compound with a suitable heterocycle having active methylene hydrogen, such as HAr(1), HAr(2), HAr(3), or HAr(4), can be accomplished by Knoevenagel type condensation reactions. It is understood by those of ordinary skill in the art that intermediates having hydroxyl groups bound thereto are sometimes formed under Knoevenagel type condensations, as shown below.

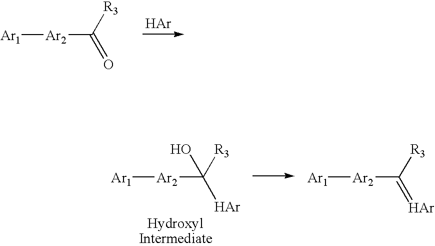

The hydroxyl groups of such intermediates are often substantially eliminated (to liberate water) during the condensation reaction, to form the desired benzylidene compound having a double bond. Nevertheless, the conditions of the reaction can be modified for the isolation or further use of such hydroxyl containing intermediates, and such embodiments are within the scope of the invention. Effective catalysts for the Knoevenagel type condensations can be selected from ammonia, primary, secondary and tertiary amines, either as the free base or the amine salt with an organic acid, such as acetic acid. Examples of catalysts include pyrrolidine, piperidine, pyridine, diethylamine and the acetate salts thereof. Inorganic catalysts can also be used for the condensation. Inorganic catalysts include, but are not limited to, titanium tetrachloride and a tertiary base, such as pyridine; and magnesium oxide or zinc oxide in an inert solvent system. This type of condensation can be strongly solvent-dependent and it is understood that routine experimentation may be necessary to identify the optimal solvent with a particular catalyst, preferable solvents include ethanol, tetrahydrofuran, dioxane or toluene; or mixtures thereof.

In an optional step, the benzylidene compounds of Formula (I) wherein the double bond is present can be reduced by a variety of methods to give a compound of Formula (I) having only a single bond, i.e., a benzyl compound having the structure

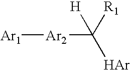

The reduction of the carbon-carbon bond of the benzylidene compound to give the reduced and/or hydrogenated benzyl compound can be accomplished by many methods known of those of ordinary skill in art, such as catalytic hydrogenation, reduction with reducing metals such as sodium or zinc in the presence of protic solvents, or via hydride reducing agents such as borohydrides, etc.

In yet other embodiments of the above method of synthesis, [symbol] represents a bond absent, and HAr has the formula:

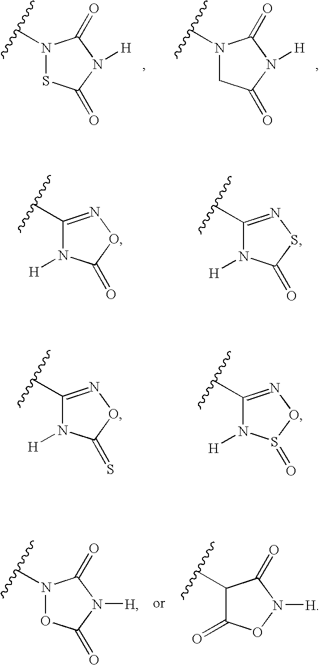

The reaction steps necessary to synthesize such heterocyclic compounds of Formula (I) are described above and in FIG. 5.

Some embodiments the invention relate to methods of making a heteroatom-linked compound of the Formula (II)

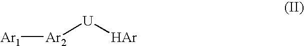

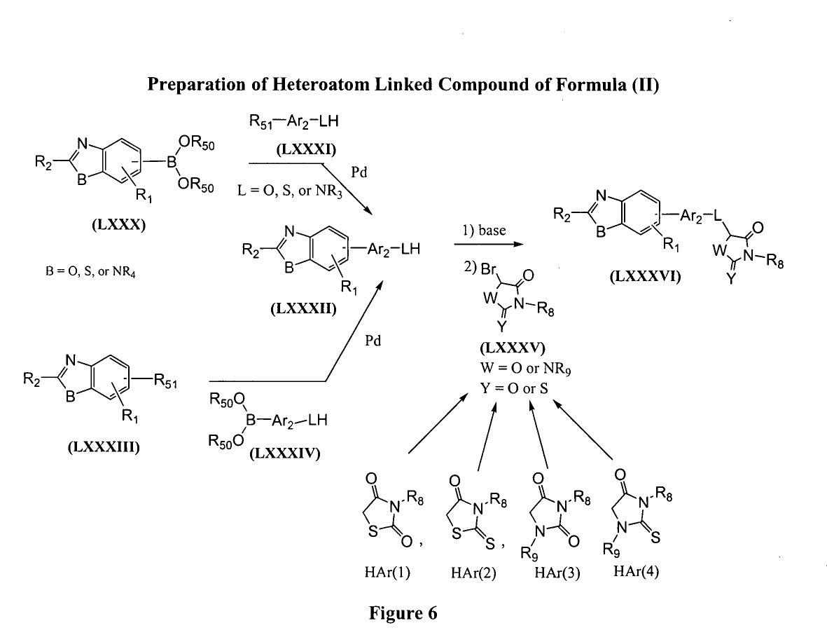

Methods for making certain heteroatom linked compounds of Formula (II) are illustrated in FIG. 6. Precursor biaryl compounds having the structure 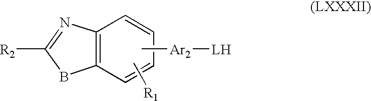
wherein L is —O—, —S—, and —NR₄, and R₁, R₂ and B have the definitions described hereinabove can be prepared, for example, by coupling a boronic acid precursor of Ar₁, such as for example the compound of Formula (LXXX), with an appropriate precursor of Ar₂ that has a “L” heteroatom substituent suitable for coupling to the five membered heterocycles of the invention. Examples of such compounds are the R₅₁—Ar₂-LH compounds having formula (LXXXI) in FIG. 6, where R₅₁ is a halide or tosylate, or preferably a bromide. Biaryl (LXXXII) can be prepared alternatively by the coupling of a boronic acid (LXXXIV) precursor of Ar₂ with a heterocyclic halide (LXXXIII) precursor of the Ar₁ benzoxazole, benzothiazole, or benzimidazole, as also shown in FIG. 6. Methods of synthesis for wide variety of substituted aromatic precursor compounds for Ar₁ and Ar₂ are disclosed elsewhere herein, or are well known to those of ordinary skill in synthetic organic chemistry arts.

Synthetic precursors of the HAr(1), HAr(2), HAr(3), or HAr(4) suitable for coupling with compound (LXXXII) can be prepared by bromination of an active methylene position of the parent heterocycles, to give the brominated heterocycle (LXXXV). For example, 5-Bromo-2-thioxo-thiazolidin-4-one can be prepared by bromination of rhodanine (HAr(2)) as described by Pujari, J. Sci. Ind. Res. 14B:398 (1955). Heterocycle (LXXXV) can then be coupled with compound (LXXXII) in the presence of base, in analogy to the reactions described by Zask et al., J. Med. Chem. 33:1418-1423 (1990), to give the desired final product heterocycles (LXXXVI).

Alternatively, brominated heterocycle (LXXXV) can be condensed with the L heteroatom of synthetic precursors of Ar₂ such as (LXXXI), and the product Ar₂-L-HAr heterocycle Suzuki coupled to an appropriate precursor of Ar₁.

Furthermore, when L=S, the sulfur linked heterocycle (LXXXVI) shown in FIG. 6 can be oxidized in a selective manner with m-chloroperbenzoic acid to provide the sulfoxide compound (L=-SO—). The sulfur atom can be further oxidized with additional m-chloroperbenzoic acid, or with hydrogen peroxide in acetic acid, as described by Zask et al., J. Med. Chem. 33:1418-1423 (1990), to provide the sulfone compounds wherein L=-SO₂—.

Biological Activity of the Compounds

Compounds described above have been found to be potent compounds in a number of in vitro biological assays that correlate to, or are representative of human diseases, especially diseases of uncontrolled cellular proliferation, including various cancers. The biological activity of the compounds described herein can be measured by testing the compounds of the invention for their ability to kill or inhibit the growth of various human tumor cell lines. Tumor cell lines that can be employed for such tests include but are not limited to known cell lines such as:
For Leukemia: CCRF-CEM, HL-60 (TB), K-562, MOLT-4, RPMI-8226, and SR. Lung Cancer: A549/ATCC, EKVX, HOP-62, HOP-92, NCI-H226, NCI-H23, NCI-H322M, NCI-H460, and NCI-H522.
Colon Cancer: COLO 205, HCC-2998, HCT-116, HCT-15, HT-29, KM-12, and SW-620.
CNS Cancer: SF-268, SF-295, SF-539, SNB-19, SNB-75, and U-251.
Melanoma: LOX-IMVI, MALME-3M, M-14, SK-MEL-2, SK-MEL-28, SK-MEL-5, UACC-257, and UACC-62.
Ovarian Cancer: IGR-OVI, OVCAR-3, OVCAR-4, OVCAR-5, OVCAR-8, and SK-OV-3.
Renal Cancer: 786-0, A-498, ACHN, CAKI-1, RXF-393, RXF-631, SN12C, TK-10, and U0-31.
Prostate Cancer: PC-3 and DU-145.
Breast Cancer: MDA-MB-468, MCF 7, MCF7/ADR-RES, MDA-MB-231/ATCC, HS578T, MDA-MB-435, MDA-N, BT-549, and T-47D.
Pancreatic Cancer: Bx-PC3.

After the compounds to be screened have been applied to one or more of the above cancer cell lines, the anti-cancer effectiveness can be gauged using a variety of assay procedures known to those of ordinary skill in the art, which include an assay that employs 3-(4,5-dimethylthiazol-2-yl)-2,5-diphenyltetrazolium bromide (“MTT”) to differentiate live cells from dead cells. The MTT assay is based on the production of a dark blue formazan product by active dehydrogenase in the mitochondria of live tumor cells (see M. C. Alley, D. A. Scudiero, A. Monks, M. L. Hursey, M. J. Czerwinski, D. L. Fine, B. J. Abbout, J. G. Mayo, R. H. Shoemaker and M. R. Boyd, Cancer Res., 48, 589, 1988). After exposure of cancer cells to the compounds to be screened for a number of days, only living cells contain active dehydrogenases, and produce dark blue formazan from MTT and are stained. the numbers of live cells can be measured by absorbance of visible light by the formazan at 595 nm. Anti-cancer activity can be reported as percent of the tumor cell growth in a culture treated with a placebo. These MTT assay procedures have an advantage over an in vivo assay with common laboratory animals such as mice, in that results are obtained within a week as opposed to requiring several months.

These MTT anti-cancer activity screening assay provides data regarding the general cytotoxicity of an individual compound. In particular, as described in the examples herein, active anticancer compounds can be identified by applying the compounds at a concentration of about 10 uM to one or more human tumor cell line cultures, such as for example leukemia, lung cancer, colon cancer, CNS cancer, melanoma, ovarian cancer, renal cancer, prostate cancer, breast cancer, or pancreatic cancer, so as to kill or inhibit cell growth of the tumor cells.

In some embodiments of the invention, the compounds of the invention are considered to be biologically active for the treatment of a particular cancer if, when they are applied to a culture of one of the above cancer cell lines at a concentration of about 10 uM, for a period of at least about 5 days, the growth of the cancer cells is inhibited, or the cancers cells killed to the extent of about 50% or more, as compared to a control not comprising the compound of the invention.

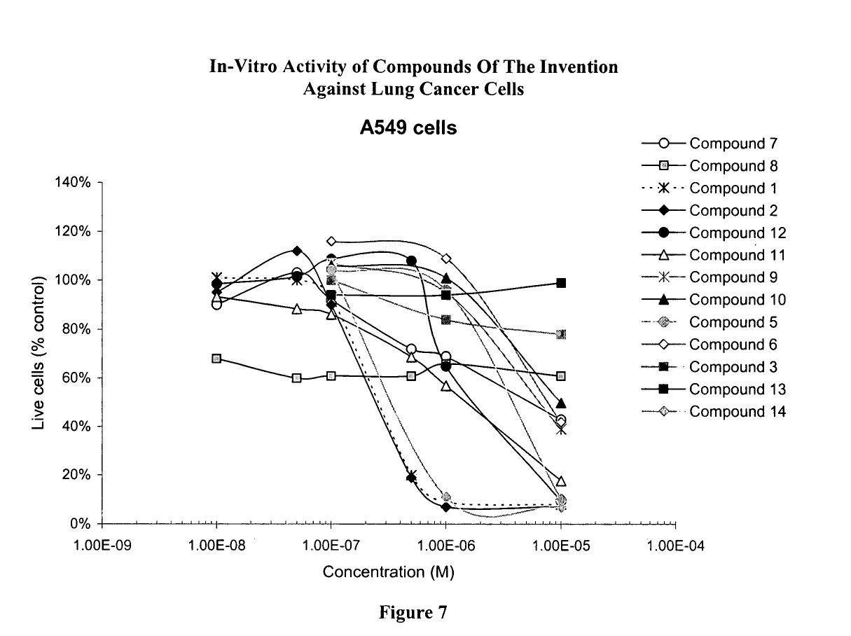

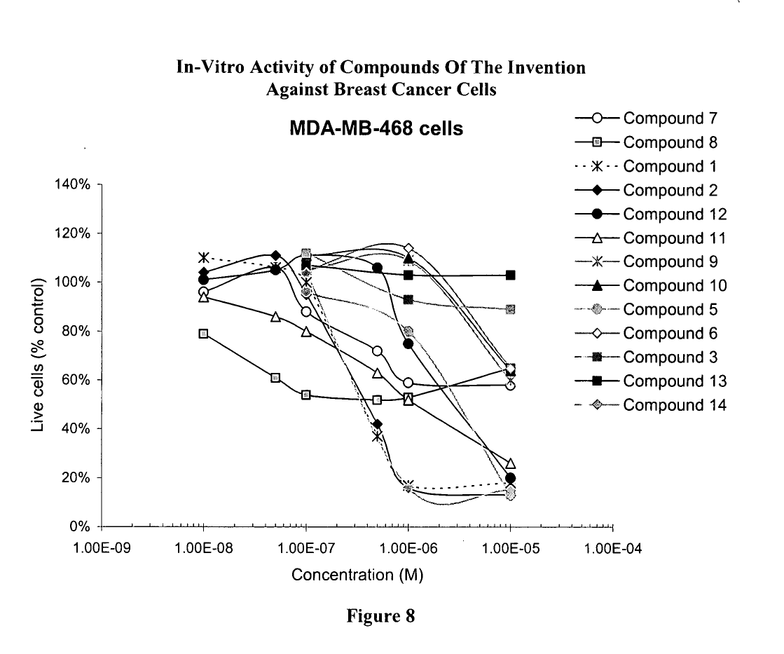

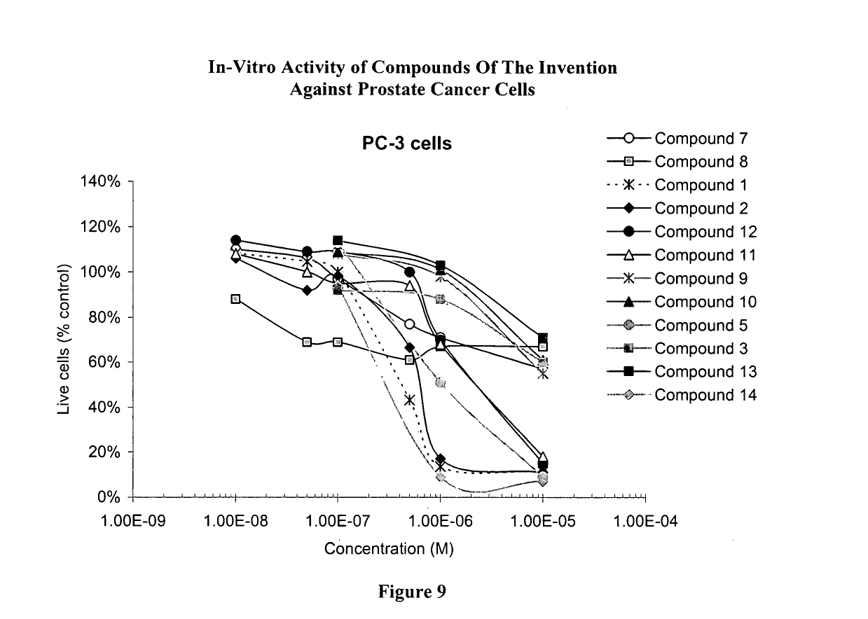

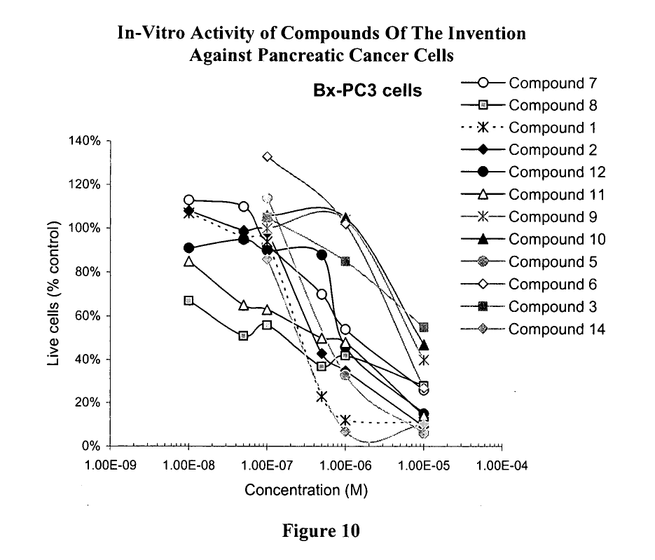

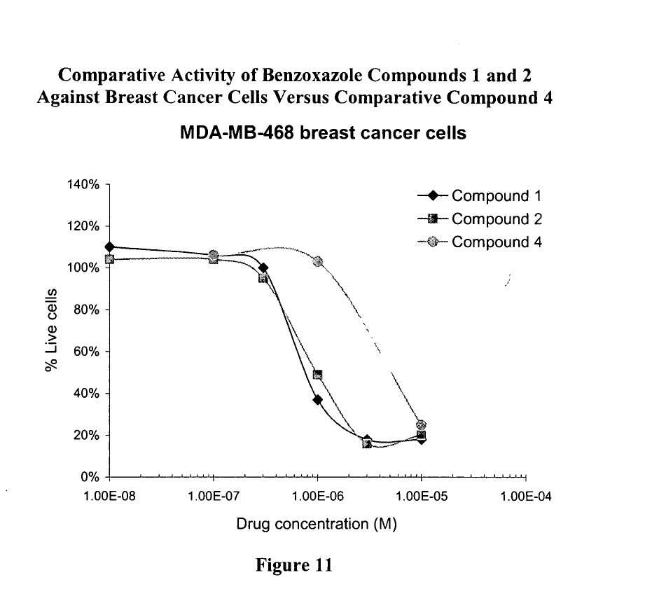

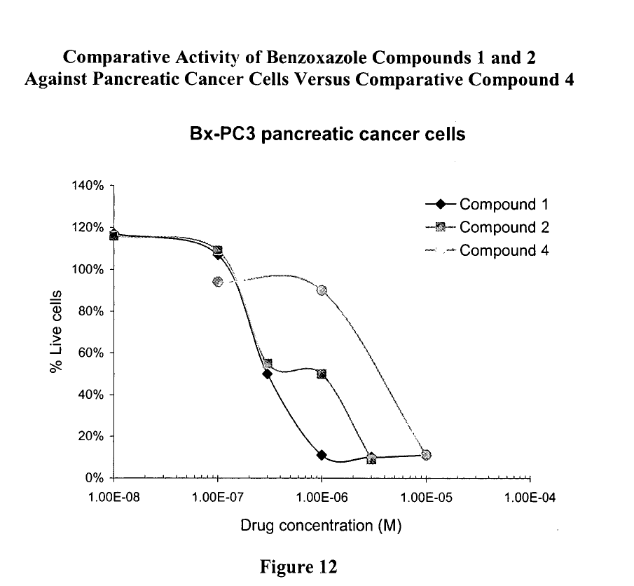

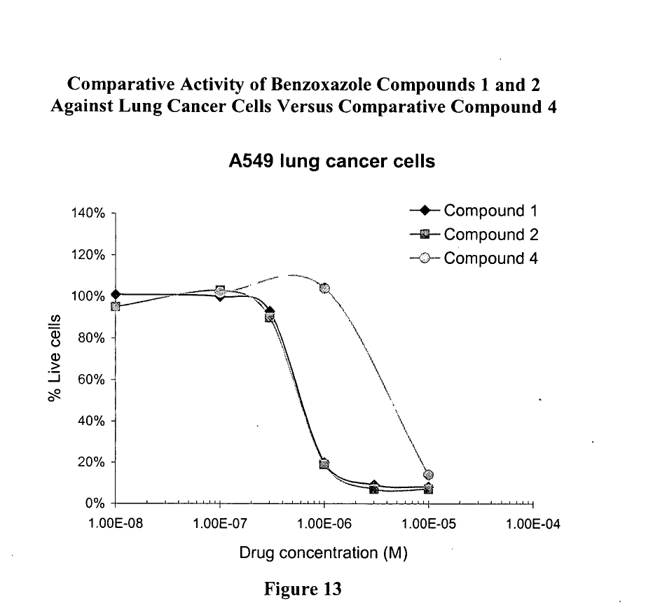

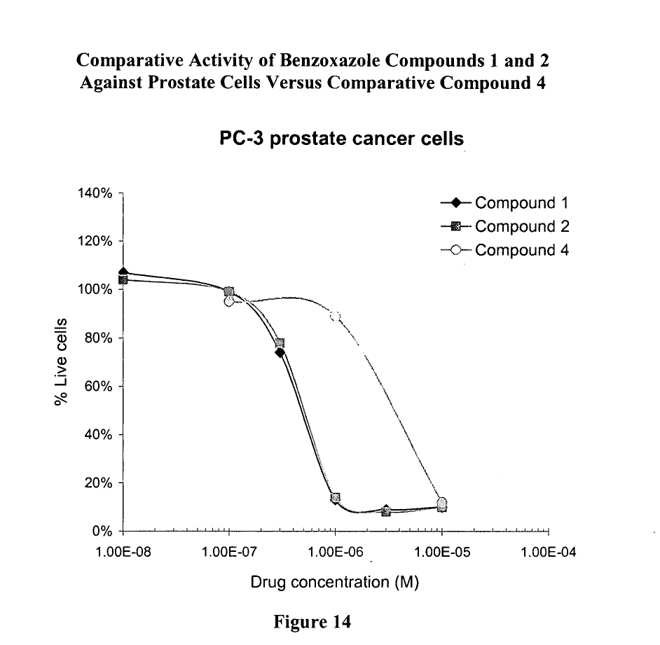

Compounds 1-14 of the invention, which exhibit significant structural variations were screened in-vitro by the procedures outline above for four human cancer cell lines, which include human cell lines for breast, prostate, lung, and pancreatic cancers. Procedures used for the screening assays are given in Examples 21 and 22, and representative results are shown in FIGS. 7-10. Results showing the unexpectedly high anti-cancer activity of compounds 1 and 2 of the invention as compared to compounds that do not comprise benzoxazole, benxothiazole, or benzimidazole rings are shown in FIGS. 11-14.

As can be seen from FIGS. 7-10, although the anticancer activity of the tested compounds varies somewhat with both the structure of the particular candidate compound and the particular cancer cell line being employed, all of compounds 1-14 exhibited significant biological activity against at least one of the four cancer cell lines tested. Compounds 1, 2, and 14 were particularly notable for their consistent and potent anti-cancer activity at low concentrations, when tested against all four cancer cell lines.

The specific biochemical mechanisms that produce the biological and/or anti-cancer activity of the compounds of the invention is not well understood, and may or may not be the same for all the compounds disclosed herein. Nevertheless, evidence has been obtained that at least some of the compounds described herein are somehow involved in or associated with the activation of the JNK signaling pathways that are associated with cell apoptosis.

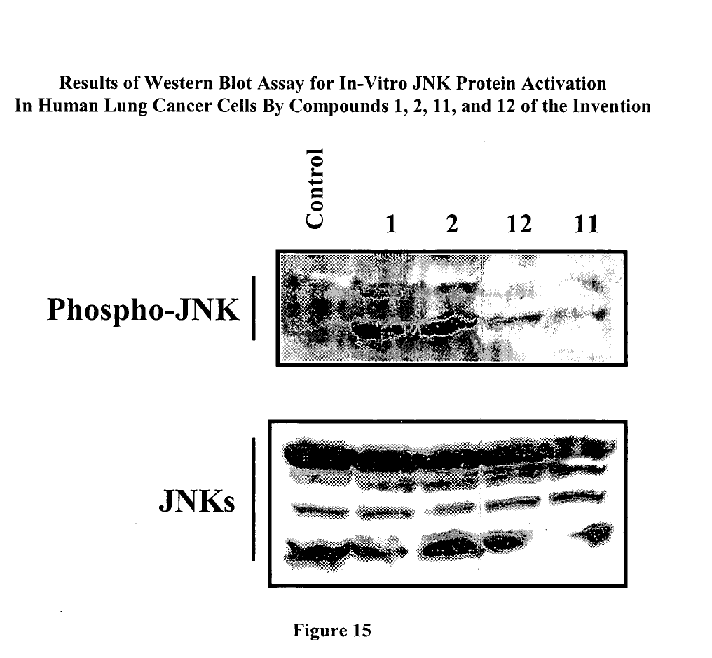

Western Blot assay techniques can be employed to detect both JNK proteins generally (whether activated or not), and for specific detection of phosphorylated JNK proteins. As described above and in the examples below, activation of the JNK signaling pathways is known to involve phosphorylation of one or more of the isoforms of the JNK proteins. As described in Example 23, a human cancer cell line was treated with some of the compounds of the invention, and the effect on JNK proteins was assayed by Western Blot assay measurements. FIG. 15 herein shows the results, which provide evidence that treatment of the cancer cells with compounds 1, 2, and 12 results in the production of phosphorylated JNK proteins. The same compounds also inhibit the growth or cause the apoptosis of many of the cancer cell lines that have been tested. Therefore, without wishing to be bound by any theory, it is believed that the compounds of the present invention are somehow associated with the activation and/or phosphorylation of the JNK signaling pathways that lead to cancer cell apoptosis.

Using the Compositions

In view of their ability to inhibit the growth of, and/or induce the apoptosis of at least some cancer cell lines in vitro, the compounds described herein can be used to prevent, alleviate or otherwise treat diseases of uncontrolled proliferation in mammals, including humans, such as cancer or precancerous diseases.

Therefore, in some embodiments, the invention relates to methods of treatment for a disease of uncontrolled cellular proliferation, wherein the method comprises administering to a mammal diagnosed as having a disease of uncontrolled cellular proliferation a compound of the invention or a pharmaceutical composition thereof comprising one or more of the compounds of the invention, in an amount that is effective to treat the disease of uncontrolled cellular proliferation. The disease of uncontrolled cellular proliferation treated can be a carcinoma, lymphoma, leukemia, or sarcoma. The types of cancer treated by methods of the invention include but are not limited to Hodgkin's Disease, meyloid leukemia, polycystic kidney disease, bladder cancer, brain cancer, head and neck cancer, kidney cancer, lung cancer, myeloma, neuroblastoma/glioblastoma, ovarian cancer, pancreatic cancer, prostate cancer, skin cancer, liver cancer, melanoma, colon cancer, cervical carcinoma, breast cancer, epithelial cancer, and leukemia. The compositions can also be used as regulators in diseases of uncontrolled proliferation and/or precancerous conditions such as cervical and anal dysplasias, other dysplasias, severe dysplasias, hyperplasias, atypical hyperplasias, and neoplasias.

The effectiveness of the methods for treating the diseases of uncontrolled cellular proliferation can vary as a function of several variables, including the specific genetic nature of disease or cancer, the details of the method of administration of the compound, the exact structure of the compounds administered, and other factors which are known to those of ordinary skill in the art.

The compounds disclosed herein can be either used singularly, or plurally, in mixtures of one or more compounds, tautomers, isomers, or enantiomers, and in pharmaceutical compositions thereof, for the treatment of mammalian diseases of uncontrolled cellular proliferatio, particularly those diseases related to humans.

Compounds disclosed herein and compositions thereof can be administered by various methods including, for example, orally, intravenously, enterally, parenterally, topically, nasally, vaginally, opthalinically, sublingually or by inhalation for the treatment of diseases related to uncontrolled proliferative diseases such as, Routes of administration and dosages known in the art can be found in Comprehensive Medicinal Chemistry, Volume 5, Hansch, C. Pergamon Press, 1990; incorporated herein by reference in its entirety.

Although the compounds described herein can be administered as pure chemicals either singularly or plurally, it is preferable to present the active ingredient as a pharmaceutical composition. Thus another embodiment of the invention is the use of a pharmaceutical composition comprising one or more compounds and/or a pharmaceutically acceptable salt thereof, together with one or more pharmaceutically acceptable carriers thereof and, optionally, other therapeutic and/or prophylactic ingredients. The carrier(s) should be “acceptable” in the sense of being compatible with the other ingredients of the composition and not overly deleterious to the recipient thereof. The pharmaceutical composition, is administered to an animal diagnosed as in need of treatment for a disease of uncontrolled cellular proliferation, in an amount effective to treat the disease of uncontrolled cellular proliferation, such as the various cancers and precancerous conditions described herein.

It will be further appreciated that the amount of the compound, or an active salt or derivative thereof (i.e. a prodrug), required for effective use in treatment of a disease of uncontrolled cellular proliferation, such as the various cancers and precancerous conditions described herein, will vary not only with the particular compound and/or salt selected but also with the route of administration, the nature of the condition being treated, and the age and condition of the patient. An effective amount of a compound provided herein is a substantially nontoxic but sufficient amount of the compound to provide a clinically useful degree inhibition of the growth or progression of the disease of uncontrolled cellular proliferation.

Though it is not possible to specify a single predetermined pharmaceutically effective amount of the compounds of the invention, and/or their pharmaceutical compositions, for each and every disease condition to be treated, determining such pharmaceutically effective amounts are within the skill of, and ultimately at the discretion of an attendant physician or clinician of ordinary skill. In some embodiments, the active compounds of the invention are administered to achieve peak plasma concentrations of the active compound of from typically about 0.1 to about 100 μM, about 1 to 50 μM, or about 2 to about 30 μM. This can be achieved, for example, by the intravenous injection of a 0.05 to 5% solution of the active ingredient, optionally in saline, or orally administered as a bolus containing about 0.5-500 mg of the active ingredient. Desirable blood levels can be maintained by continuous infusion to provide about 0.01-5.0 mg/kg/hr or by intermittent infusions containing about 0.4-15 mg/kg of the active compounds of the invention.

Pharmaceutical compositions include those suitable for oral, enteral, parental (including intramuscular, subcutaneous and intravenous), topical, nasal, vaginal, ophthalinical, sublingually or by inhalation administration. The compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The compounds of the invention can have oral bioavailability as exhibited by blood levels after oral dosing, either alone or in the presence of an excipient. Oral bioavailability allows oral dosing for use in chronic diseases, with the advantage of self-administration and decreased cost over other means of administration. Pharmaceutical compositions suitable for oral administration can be presented as discrete unit dosage forms such as hard or soft gelatin capsules, cachets or tablets each containing a predetermined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art., e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For topical administration to the epidermis, the compounds can be formulated as ointments, creams or lotions, or as the active ingredient of a transdemial patch. Suitable transdermal delivery systems are disclosed, for example, in Fisher et al. (U.S. Pat. No. 4,788,603, incorporated herein by reference) or Bawas et al. (U.S. Pat. Nos. 4,931,279, 4,668,504 and 4,713,224; all incorporated herein by reference). Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. No. 4,140,122, 4383,529, or 4,051,842; incorporated herein by reference.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention can also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, one of skill in the art understands how to extrapolate in vivo data obtained in a model organism, such as athymic nude mice inoculated with human tumor cell lines, to another mammal, such as a human. These extrapolations are not simply based on the weights of the two organisms, but rather incorporate differences in metabolism, differences in pharmacological delivery, and administrative routes. Based on these types of considerations, a suitable dose will, in alternative embodiments, typically be in the range of from about 0.5 to about 10 mg/kg/day, or from about 1 to about 20 mg/kg of body weight per day, or from about 5 to about 50 mg/kg/day.

The desired dose can conveniently be presented in a single dose or as divided doses administered at appropriate intervals, for example, as two, three, four or more sub-doses per day. The sub-dose, as necessary by one skilled in the art, can itself be further divided, e.g., into a number of discrete loosely spaced administrations.

One skilled in the art will recognize that dosage and dosage forms outside these typical ranges can be tested and, where appropriate, be used in the methods of this invention.

Combinations with Other Active Agents

According to another aspect of the invention, pharmaceutical compositions of matter useful for the treatment of cancer are provided that contain, in addition to the aforementioned compounds, an additional therapeutic agent. Such agents can be chemotherapeutic agents, ablation or other therapeutic hormones, antineoplastic agents, monoclonal antibodies useful against cancers and angiogenesis inhibitors. The following discussion highlights some agents in this respect, which are illustrative, not limitative. A wide variety of other effective agents also can be used.

Among hormones which can be used in combination with the present inventive compounds, diethylstilbestrol (DES), leuprolide, flutamide, cyproterone acetate, ketoconazole and amino glutethimide.

Among antineoplastic and anticancer agents that can be used in combination with the inventive compounds, 5-fluorouracil, vinblastine sulfate, estramustine phosphate, suramin and strontium-89. Other chemotherapeutics useful in combination and within the scope of the present invention are buserelin, chlorotranisene, chromic phosphate, cisplatin, cyclophosphamide, dexamethasone, doxorubicin, estradiol, estradiol valerate, estrogens conjugated and esterified, estrone, ethinyl estradiol, floxuridine, goserelin, hydroxyurea, melphalan, methotrexate, mitomycin, prednisone and tamoxifen.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as can be applied to the essential features hereinbefore set forth, and as follows in the scope of the appended claims.

### EXAMPLES

The following examples are given merely to illustrate the invention and are not intended to be limiting in any manner. For the purposes of this document, the compounds individually disclosed in the following Examples 1-20 can be referred to in shorthand by the number of the example. For example, as shown immediately below, Example discloses a synthesis of a particular compound, which is referred to elsewhere herein as Example 1.

### Example 1

### 5-[6-(7-Adamantan-1-yl-2-methyl-benzoxazol-5-yl)-pyridin-3-ylmethylene]-thiazolidine-2,4-dione

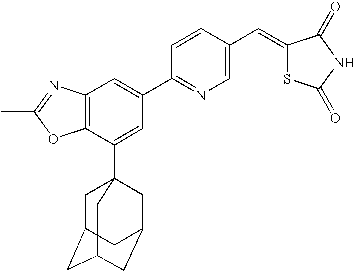

A solution of toluene (75 mL), piperidine (0.161 mL, 0.30 eq), acetic acid (0.93 mL, 0.3 eq), 6-(7-Adamantan-1-yl-2-methyl-benzooxazol-5-yl)-pyridin-3-carbaldehyde (2.02 g, 5.43 mmol) and 2,4-thiazolidinedione (700 mg, 5.96 mmol) was heated at reflux overnight under an argon atmosphere. The reaction mixture was concentrated to half volume and the yellow solid collected and washed with toluene (5 mL) and hexane (15 mL). The solid was further recrystallized from ethanol/water and dried under high vacuum to afford 1.37 g (54%) of 5-[6-(7-Adamantan-1-yl-2-methyl-benzooxazol-5-yl)-pyridin-3-ylmethylene]-thiazolidine-2,4-dione, mp>360° C. ¹H NMR (300 MHz; DMSO-d₆): 1.79 (s, 6H), 2.12 (s, 9H), 2.64 (s, 3H), 7.82 (s, 1H), 7.96 (dd, J₁=2.4 Hz, J₂=8.7 Hz, 1H), 8.02 (d, J=2.4 Hz, 1H), 8.18 (d, J=8.4 Hz, 1H), 8.21 (d, J=1.8 Hz, 1H), 8.87 (d, J=2.4 Hz, 1H), 12.66 (brs, 1H).

The intermediate 6-(7-Adamantan-1-yl-2-methyl-benzoxazol-5-yl)-pyridin-3-carbaldehyde was prepared as follows:

a. 6-(7-adamantan-1-yl-2-methyl-benzoxazol-5-yl)-pyridin-3-carbaldehyde.

To a solution of 2-Adamantan-1-yl-6-amino-4-(5-[1,3]dioxolan-2-yl-pyridin-2-yl)-phenol (6.48 g, 16.51 mol) in toluene (400 mL) was added acetic anhydride (2.03 mL, 1.3 eq) and p-toluenesulfonic acid (3.3 g, 1.05 eq) and the solution refluxed for 8 hrs. The water was removed using a Dean-Stark apparatus. After cooling the solution was diluted with ethylacetate and washed successively with saturated aqueous NaHCO₃ water and brine, dried (MgSO₄), filtered and evaporated. The residue was further chromatographed on silica gel (eluent: hexane:ethyl acetate, 7:3) to give 6-(7-adamantan-1-yl-2-methyl-benzooxazol-5-yl)-pyridin-3-carbaldehyde (2.02 g, 33%). 1.86 (s, 6H), 2.1 (s, 3H), 2.21 (s, 6H), 7.93 (d, J=8.1 Hz, 1H), 8.02 (d, J=0.9 Hz, 1H), 8.13 (d, J=0.9 Hz, 1H), 8.23 (dd, J₁=2.1 Hz, J₂=8.7 Hz, 1H), 9.13 (d, J=1.8 Hz, 1H), 10.14 (s, 1H).

b. 2-Adamantan-1-yl-6-amino-4-(5-[1,3]dioxolan-2-yl-pyridin-2-yl)-phenol.

To a solution of 2-Adamantan-1-yl-4-(5-[1,3]dioxolan-2-yl-pyridin-2-yl)-6-nitro-phenol (7.40 g, 17.51 mmol) in 500 mL of EtOH:CH₂Cl₂ (4:1) was added ammonium formate (5.52 g, 5 eq) and Pd/C (10%, 750 mg) and the solution refluxed for 2 hrs. The solution was cooled to room temperature, filtered and evaporated. The residue was dissolved in ethyl acetate and washed successively with water and brine, dried over anhydrous magnesium sulfate, filtered and evaporated to give 6.84 g of 2-Adamantan-1-yl-6-amino-4-(5-[1,3]dioxolan-2-yl-pyridin-2-yl)-phenol (100%). ¹H NMR (300 MHz; CDCl₃): δ 1.79 (s, 6H), 2.10 (s, 3H), 2.17 (s, 6H), 4.1 (m, 4H), 5.87 (s, 1H), 7.49 (s, 2H), 7.65 (d, J=8.4 Hz, 1H), 7.78 (dd, J₁=2.4 Hz, J₂=8.1 Hz, 1H), 8.70 (d, J=1.8 Hz, 1H).

C. 2-Adamantan-1-yl-4-(5-[1,3]dioxolan-2-yl-pyridin-2-yl)-6-nitro-phenol.

A mixture of 6-(3-Adamantan-1-yl-4-hydroxy-5-nitro-phenyl)-pyridine-3-carbaldehyde (6.74 g, 17.81 mmol), ethylene glycol (3 mL, 3 eq) and p-toluenesulfonic acid (68 mg, 0.02 eq) in toluene (300 mL) was refluxed for 2 hrs. The water was removed using a Dean-Stark apparatus. After cooling the solution was washed with water. The aqueous layer was further ectacted with ethylacetate. The organics were combined, dried (MgSO₄), filtered and evaporated to give 7.40 g of 2-Adamantan-1-yl-4-(5-[1,3]dioxolan-2-yl-pyridin-2-yl)-6-nitro-phenol (98%) as a yellow solid. ¹H NMR (300 MHz; CDCl₃): δ 1.81 (s, 6H), 2.10 (s, 3H), 2.22 (s, 6H), 4.1 (m, 4H), 5.91 (s, 1H), 7.73 (d, J=8.4 Hz, 1H), 7.86 (dd, J₁=2.4 Hz, J₂=8.1 Hz, 1H), 8.30 (d, J=2.4 Hz, 1H), 8.60 (dd, J₁=0.9 Hz, J₂=2.4 Hz, 1H), 8.76 (d, J=1.8 Hz, 1H), 11.74 (s, 1H).

d. 6-(3-Adamantan-1-yl-4-hydroxy-5-nitro-phenyl)-pyridine-3-carbaldehyde.

To a solution of 6-(3-Adamantan-1-yl-4-hydroxy-phenyl)-pyridine-3-carbaldehyde in dichloromethane (500 mL) was added dropwise over a period of 0.5 hr nitronium tetrafluoroborate (NO₂—BF₄, 0.5 M in sulfolane, 200 mL, 3.5 eq) and the reaction stirred at room temperature for 2 hrs. The solution was washed with water and brine, dried over anhydrous magnesium sulfate, filtered, and evaporated. The residue was recrystalized from ethanol-water to give 6.74 g of 6-(3-Adamantan-1-yl-4-hydroxy-5-nitro-phenyl)-pyridine-3-carbaldehyde. ¹H NMR (300 MHz; CDCl₃): δ 1.82 (s, 6H), 2.15 (s, 3H), 2.23 (s, 6H), 7.90 (d, J=8.4 Hz, 1H), 8.24 (dd, J₁=2.4 Hz, J₂=8.1 Hz, 1H), 8.39 (d, J=2.1 Hz, 1H), 8.73 (d, J=2.4 Hz, 1H), 9.12 (dd, J₁=0.9 Hz, J₂=2.1 Hz, 1H), 10.14 (s, 1H), 11.82 (s, 1H).

e. 6-(3-Adamantan-1-yl-4-hydroxy-phenyl)-pyridine-3-carbaldehyde.

To a solution of 6-[3-adamantan-1-yl-4-(t-butyldimethyl-silanyloxy)-phenyl]-pyridine-3-carbaldehyde (15.95 g, 35.6 mmol) in 400 mL of dry THF cooled to 0° C. was added dropwise 43 mL of 1.0 M solution of tetrabutylammonium floride in THF. The solution was brought to room temperature over a period of 2 hrs. The mixture was washed with water and brine, dried (MgSO₄), filtered and evaporated. The resulting solid was dried under high vacuum to give 12.16 g of 6-(3-Adamantan-1-yl-4-hydroxy-phenyl)-pyridine-3-carbaldehyde (100%). ¹H NMR (300 MHz; CDCl₃): δ 1.79 (s, 6H), 2.09 (brs, 3H), 2.20 (s, 6H), 5.98 (brs, 1H), 6.86 (d, J=8.1 Hz, 1H), 7.76 (dd, J₁=8.1, J₂=2.4 Hz, 1H), 8.01 (d, J=2.4 Hz, 1H), 8.17 (dd, J₁=8.1, J₂=2.4 Hz, 1H), 9.06 (d, J=2.4 Hz, 1H), 10.09 (s, 1H).

f. 6-[3-Adamantan-1-yl-4-(t-butyldimethyl-silanyloxy)-phenyl]-pyridine-3-carbaldehyde.

A mixture of 6-bromopyridine-3-carboxaldehyde (15.00 g, 0.0806 mol), 3-adamantan-1-yl-4-t-butyldimethylsilanyloxyphenyl boronic acid (37.39 g, 0.09677 mmol) and sodium carbonate (1.719 g, 12.44 mmol) in 750 mL of toluene:EtOH (4:1) and 75 mL of water was degassed with argon for 30 minutes. Tetrakis(triphenyl-phosphine)palladium(0) (2.335 g, 0.00202 mmol, 0.025 eq) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 9:1) to give 24.69 g of 6-[3-adamantan-1-yl-4-(t-butyldimethyl-silanyloxy)-phenyl]-pyridine-3-carbaldehyde (68%). ¹H NMR (300 MHz; CDCl₃): δ 0.39 (s, 6H), 1.06 (s, 9H), 1.79 (brs, 6H), [2.11 (brs), 2.19 (s), 9H], 6.91 (d, J=8.4 Hz, 1H), 7.75-7.85 (m, 2H), 8.04 (d, J=2.1 Hz, 1H), 8.16 (dd, J₁=8.4, J₂=2.1 Hz, 1H), 9.06 (d, J=2.1 Hz, 1H), 10.09 (s, 1H).

g. 3-Adamantan-1-yl-4-t-butyldimethylsilanyloxyphenyl boronic acid.

To a solution of n-BuLi (142.4 mL, 2.5 M, 0.356 mmol, 1.5 eq) in THF (1.1 L) cooled to −78° C. under an atmosphere of argon was added a solution of 3-adamantan-1-yl-4-t-butyldimethylsilanyloxy bromobenzene (100.0 g, 0.237 mol) in THF (200 mL) dropwise over 30 minutes. After stirring for 1 hour at −78° C., triisopropylborate (133.9 g, 0.712 mol, 164 mL, 3.0 eq) was added dropwise over 30 minutes and the cold bath was removed. The mixture was stirred for 45 minutes (internal temperature <0° C.), 200 mL of saturated NH₄Cl was added and the mixture was stirred overnight. The mixture was diluted with ethyl acetate and the layers separated, the aqueous layer was extracted once with ethyl acetate and the two organic layers combined. The resulting organic layer was washed with water, brine and dried (MgSO₄). The mixture was filtered, evaporated and the residue stirred in hexane. The resulting white suspension was filtered and the white solid dried under high vacuum to afford 54.7 g of 3-adamantan-1-yl-4-t-Butyl-dimethyl-silanyloxy-phenylboronic acid (59%). Additional material can be obtained from the hexane filtrate using silica gel chromatography. ¹H NMR (300 MHz; CDCl₃): δ 0.40 (s, 6H), 1.07 (s, 9H), 1.82 (brs, 6H), 2.11 (brs, 3H). 2.22 (s, 6H), 6.91 (d, J=7.8 Hz, 1H), 7.92 (dd, J₁=7.8 Hz, J₂=1.5 Hz, 1H), 8.16 (d, J=1.5 Hz, 1H).

h. 3-Adamantan-1-yl-4-t-butyldimethylsilanyloxy bromobenzene.

A 2.0 L three-neck flask attached with a power-stirrer was charged with 2-adamantan-1-yl-4-bromophenol (102.8 g, 0.334 mol, 1.0 eq), DMAP (3.67 g, 0.0301 mol), anhydrous DMF (1.0 L) and triethylamine (76.1 g, 0.753 mol, 1.25 eq). Stirring was initiated and to the resulting solution at room temperature was added t-butyl-dimethylsilyl chloride (99.8 g, 0.662 mmol, 1.10 eq). The resulting mixture was allowed to stir overnight, poured into water, and extracted with diethyl ether (2X). The combined organics were washed successively with water and brine, dried over anhydrous magnesium sulfate, filtered, and evaporated. The residue was purified on silica gel (hexane) to give 179 g (70%) of 3-adamantan-1-yl-4-t-butyldimethylsilanyloxybromobenzene as a white powder. ¹H NMR (300 MHz; CDCl₃): δ 0.33 (s, 6H), 1.03 (s, 9H), 1.75 (brs, 6H), 2.06 (s, 9H), 6.65 (d, J=8.4 Hz, 1H), 7.14 (dd, J₁=8.4 Hz, J₂=2.1 Hz, 1H), 7.29 (d, J=2.1 Hz, 1H).

i. 2-Adamantan-1-yl-4-bromophenol.

A 2.0 L three-neck flask attached with a power-stirrer was charged with 4-bromophenol (340.8 g, 1.97 mmol) and 1-adamantanol (300.0 g, 1.97 mmol) in 1.0 L of anhydrous CH₂Cl₂ at room temperature. Stirring was initiated and once all the reagents were solubilized then concentrated H₂SO₄ (105 mL, 193.2 g, 1.97 mmol, 1.0 eq) was added dropwise over 15-30 minutes. After approximately 1.0 hour a suspension resulted and the reaction was allowed to continue for a total of 24 hours. The suspension was carefully poured into ice water and neutralized with solid NaHCO₃. The resulting layers were separated and the aqueous layer extracted with CH₂Cl₂ (2X). The combined organics were washed with brine, dried (MgSO₄) and filtered. The solvent was removed under reduced pressure and the resulting solid was suspended in a minimal amount of hexanes. After stirring at room temperature for an hour the solid was collected via filtration and dried under reduced pressure to give 495.0 g (77%) of 2-adamantan-1-yl-4-bromophenol as a white solid. ¹H NMR (300 MHz; CDCl₃): δ 1.77 (s, 6H), 2.08 (s, 9H), 4.81 (s, 1H), 6.53 (d, J=8.4 Hz, 1H), 7.14 (dd, J=8.7 Hz, J₂=2.4 Hz, 1H), 7.29 (d, J=2.4 Hz, 1H).

### Example 2

### 5-[6-(7-Adamantan-1-yl-benzoxazol-5-yl)-pyridin-3-ylmethylene]-thiazolidine-2,4-dione.

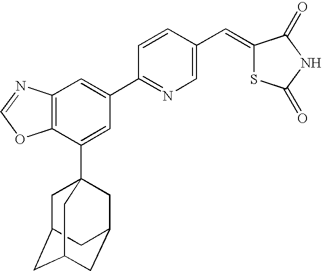

Prepared in a similar manner as described in Example 1 using 6-(7-Adamantan-1-yl-benzoxazol-5-yl)-pyridin-3-carbaldehyde mp 311-312° C., ¹H NMR (300 MHz; CDCl₃): δ 1.86 (br. s, 6H), 2.18 (br. s, 3H), 2.22 (br. s, 6H), 7.81 (s, 1H), 7.92 (m, 2H), 8.06 (s, 1H), 8.22 (s, 1H), 8.25 (s, 1H), 8.84 (s, 1H).

The intermediate 6-(7-Adamantan-1-yl-benzoxazol-5-yl)-pyridin-3-carbaldehyde was prepared as follows:

a. 6-(7-Adamantan-1-yl-benzoxazol-5-yl)-pyridin-3-carbaldehyde.

To a solution of 7-Adamantan-1-yl-5-(5-[1,3]dioxolan-2-yl-pyridin-2-yl)-benzoxazole (1.55 g, 3.85 mmol) dissolved in a mixture of acetone (120 mL) and water (20 mL) was added pyridinium p-toluene sulfonate and the reaction mixture was heated at reflux for 12 hrs. After cooling the solution was quenched into saturated aqueous NaHCO₃ and extracted with ethyl acetate. The organic layer was further washed with water and brine, dried (MgSO₄), filtered and evaporated. The residue was chromatographed on silica gel (EtOAc:Hexane 30 to 60%) to give 6-(7-Adamantan-1-yl-benzooxazol-5-yl)-pyridin-3-carbaldehyde. ¹H NMR (300 MHz; CDCl₃): δ 1.88 (br. s, 6H), 2.20 (br. s, 3H), 2.24 (br. s, 6H), 7.98 (d, J=8.1 Hz, 1H), 8.11 (d, J=1.5 Hz, 1H), 8.19 (s, 1H), 8.26 (dd, J=2.1 Hz, J₂=8.4 Hz, 1H), 8.30 (d, J=1.8 Hz, 1H), 9.16 (d, J=1.5 Hz, 1H), 10.17 (s, 1H).

b. 7-Adamantan-1-yl-5-(5-[1,3]dioxolan-2-yl-pyridin-2-yl)-benzoxazole.

To a solution of 2-Adamantan-1-yl-6-amino-4-(5-[1,3]dioxolan-2-yl-pyridin-2-yl)-phenol (Example 1b)(2 g, 5.09 mmol) in toluene (60 mL) was added 1,3,5-triazine (826 mg, 2.0 eq) and the solution refluxed for 12 hrs. After cooling the solution was quenched into saturated aqueous NaHCO₃ and extracted with ethyl acetate. The organic was further washed with water and brine, dried (MgSO₄), filtered and evaporated. The residue was chromatographed on silica gel (EtOAc:Hexane 4:6) to give 7-Adamantan-1-yl-5-(5-[1,3]dioxolan-2-yl-pyridin-2-yl)-benzoxazole. ¹H NMR (300 MHz; CDCl₃): δ 1.85 (br. s, 6H), 2.17 (br. s, 3H), 2.23 (br. s, 6H), 4.12 (m, 2H), 5.93 (s, 1H), 7.77 (d, J=8.1 Hz, 1H), 7.88 (dd, J₁=2.1 Hz, J₂=8.1 Hz, 1H), 7.99 (d, J=1.8 Hz, 1H), 8.15 (s, 1H), 8.17 (d, J=1.8 Hz, 1H), 8.79 (d, J=1.5 Hz, 1H).

### Example 3

### 5-[6-(7-Adamantan-1-yl-2-phenyl-benzoxazol-5-yl)-pyridin-3-ylmethylene]-thiazolidine-2,4-dione

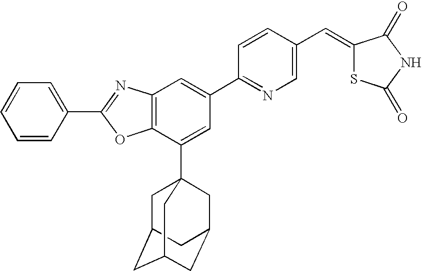

Prepared in a similar manner as described in Example 1 using 6-(7-Adamantan-1-yl-2-phenyl-benzoxazol-5-yl)-pyridine-3-carbaldehyde. mp 352-353° C., ¹H NMR (300 MHz; DMSO-d₆): δ 1.88 (br. s, 6H), 2.19 (br. s, 3H), 2.24 (br. s, 6H), 7.65-7.69 (m, 3H), 7.89 (s, 1H), 8.04 (dd, J₁=2.4, J₂=8.7 Hz, 1H), 8.16 (d, J=1.8 Hz, 1H), 8.23-8.30 (m, 3H), 8.41 (d, J=1.5 Hz, 1H), 8.95 (d, J=2.4 Hz, 1H).

The intermediate 6-(7-Adamantan-1-yl-2-phenyl-benzoxazol-5-yl)-pyridine-3-carbaldehyde was prepared as follows:

a. 6-(7-Adamantan-1-yl-2-phenyl-benzoxazol-5-yl)-pyridine-3-carbaldehyde.

Prepared in a similar manner as described in Example 2a using 7-Adamantan-1-yl-5-(5-[1,3]dioxolan-2-yl-pyridin-2-yl)-2-phenyl-benzooxazole. ¹H NMR (300 MHz; DMSO-d6): δ 1.92 (br. s, 6H), 2.23 (br. s, 3H), 2.31 (br. s, 6H), 7.56-7.60 (m, 3H), 7.97 (d, J=8.1 Hz, 1H), 8.09 (d, J=0.6 Hz, 1H), 8.24-8.31 (m, 4H), 9.15 (dd, J₁=0.6 Hz, J₂=2.1 Hz, 1H), 10.16 (s, 1H).

b. 7-Adamantan-1-yl-5-(5-[1,3]dioxolan-2-yl-pyridin-2-yl)-2-phenyl-benzoxazole.

To a solution of 2-Adamantan-1-yl-6-amino-4-(5-[1,3]dioxolan-2-yl-pyridin-2-yl)-phenol (Example 1b)(2 g, 5.09 mmol) in toluene (120 mL) was added benzoyl chloride (0.77 mL, 1.3 eq) and the solution refluxed for 1 hr. p-Toluene sulfonic acid (1.01 g, 1.05 eq) was added to the reaction mixture and the solution refluxed for 12 hrs using a Dean-Stark trap. After cooling the solution was quenched into saturated aqueous NaHCO₃ and extracted with ethyl acetate. The organic was further washed with water and brine, dried (MgSO₄), filtered and evaporated to give 2.29 g of 7-Adamantan-1-yl-5-(5-[1,3]dioxolan-2-yl-pyridin-2-yl)-2-phenyl-benzooxazole (94%). ¹H NMR (300 MHz; CDCl₃): δ 1.89 (br. s, 6H), 2.20 (br. s, 3H), 2.30 (br. s, 6H), 4.12 (m, 2H), 5.93 (s, 1H), 7.55-7.57 (m, 3H), 7.77 (d, J=8.1 Hz, 1H), 7.88 (dd, J₁=2.1 Hz, J₂=7.8 Hz, 1H), 7.97 (s, 1H), 8.14 (s, 1H), 8.26-8.30 (m, 2H), 8.79 (d, J=1.5 Hz, 1H).

### Comparative Example 4

### 5-[6-(7-Adamantan-1-yl-benzo[1,3]dioxol-5-yl)-pyridin-3-ylmethylene]-thiazolidine-2,4-dione.

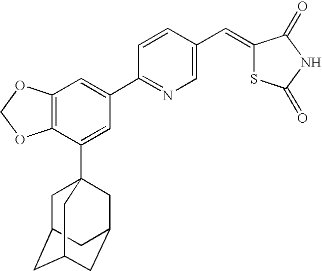

Prepared in a similar manner as described in Example 1 using 6-(7-Adamantan-1-yl-benzo[1,3]dioxol-5-yl)-pyridin-3-carbaldehyde. mp 310-314° C., ¹H NMR (300 MHz; DMSO-d₆): δ 1.76 (s, 6H), 2.05 (bs, 9H), 6.08 (s, 2H), 7.58 (d, J=1.5 Hz, 1H), 7.67 (d, J=1.8 Hz, 1H), 7.85 (s, 1H), 7.95 (dd, 1H, J₁=8.4 Hz, J₂=2.4 Hz), 8.08 (d, J=8.7 Hz, 1H), 8.85 (d, J=2.1 Hz, 1H), 12.71 (s, 1H).

The intermediate 6-(7-Adamantan-1-yl-benzo[1,3] dioxol-5-yl)-pyridin-3-carbaldehyde was prepared as follows:

a. 6-(7-Adamantan-1-yl-benzo[1,3]dioxol-5-yl)-pyridin-3-carbaldehyde.

A mixture of 3-(1-adamantyl)-4,5-methylenedioxy-1-bromobenzene (1.5 g, 5.00 mmol), 6-bromopyridine-3-carboxaldehyde (0.8 g, 4.3 mmol) and sodium carbonate (1.13 g, 10.7 mmol) in toluene (20 mL), ethanol (4 mL) and water (2.5 mL) was degassed with argon for 30 minutes. Tetrakis(triphenylphosphine)palladium(0) (0.25 g, 0.215 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 9:1) to give 1.2 g of 6-(7-Adamantan-1-yl-benzo[1,3]dioxol-5-yl)-pyridin-3-carbaldehyde. ¹H NMR (300 MHz; CDCl₃): δ 1.79 (s, 6H), 2.08 (s, 9H), 6.01 (s, 2H), 7.35 (d, J=1.5 Hz, 1H), 7.51 (s, 1H), 7.87(d, J=8.1 Hz, 1H), 8.31 (m, J=1H), 9.22 (s, 1H), 9.22 (s, 1H).

b. 3-(1-Adamantyl)-4,5-methylenedioxy-1-bromobenzene.

To a mixture of 3,4-methylenedioxy-1-bromobenzene (5.00 g, 24.87 mmol) and 1-adamantanol (3.79 g, 24.87 mmol) in CH₂Cl₂ (50 mL) under an atmosphere of argon was added sulfuric acid (2.0 mL) at room temperature. After stirring for 3 days the resulting mixture was diluted with CH₂Cl₂ and washed with water. The aqueous layer was extracted with CH₂Cl₂ and the combined organics were washed successively with water, brine and dried (MgSO₄). The mixture was filter, evaporated and the residue purified on silica gel (hexane) to give 4.41 g of 3-(1-adamantyl)-4,5-methylenedioxy-1-bromobenzene (53%) as a white solid, mp 135.5-136.0° C.

### Example 5

### 5-[4-(7-Adamantan-1-yl-2-methyl-benzoxazol-5-yl)-benzylidene]-thiazolidine-2,4-dione.

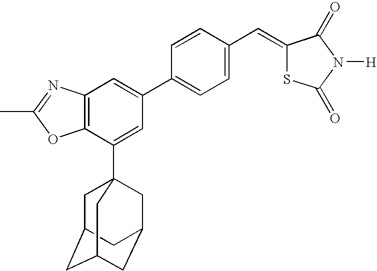

Prepared in a similar manner as described in Example 1 using 4-(7-Adamantan-1-yl-2-methyl-benzoxazol-5-yl)-benzaldehyde. mp 354-360° C., ¹H NMR (300 MHz; DMSO-d6): δ 1.81 (s, 6H), (2.13 (s), 2.16 (s), 9H), 2.66 (s, 3H), 7.46 (s, 1H), 7.69 (d, J=8.0 Hz, 1H), 7.85 (s, 2H), 7.90 (d, J=8.0 Hz, 2H), 12.65 (brs, 1H).

### Example 6

### 5-[3-(7-Adamantan-1-yl-2-methyl-benzoxazol-5-yl)-benzylidene]-thiazolidine-2,4-dione

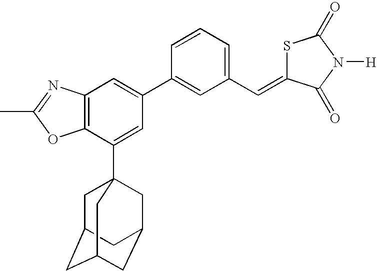

Prepared in a similar manner as described in Example 1 using 3-(7-Adamantan-1-yl-2-methyl-benzoxazol-5-yl)-benzaldehyde. mp 355-358° C., ¹H NMR (300 MHz; DMSO-d6): δ 1.81 (s, 6H), [2.12 (s), 2.16 (s), 9H], 2.66 (s, 3H), 7.43 (s, 1H), 7.55 (d, J=7.5 Hz, 1H), 7.62 (t, J=7.5 Hz, 1H), 7.80-7.83 (m, 2H), 7.95 (m, 2H), 12.66 (brs, 1H).

### Example 7

### 5-[4-(5-Adamantan-1-yl-2-methyl-benzoxazol-7-yl)-benzylidene]-thiazolidine-2,4-dione

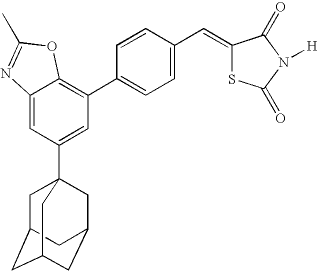

Prepared in a similar manner as described in Example 1 using 4-(5-Adamantan-1-yl-2-methyl-benzoxazol-7-yl)-benzaldehyde. mp>360° C., ¹H NMR (300 MHz; DMSO-d6): δ 1.79 (br t, 6H), 2.00 (br d, 6H), 2.12 (br s, 3H), 2.65 (s, 3H), 7.60 (d, J=1.8 Hz, 1H), 7.62 (d, J=1.8 Hz, 1H), 7.74 (d, J=8.7 Hz, 2H), 7.86 (s, 1H), 8.05 (d, J=8.1 Hz, 2H), 8.24 (s, 1H).

The intermediate 4-(5-Adamantan-1-yl-2-methyl-benzoxazol-7-yl)-benzaldehyde was prepared as follows:

a. 4-(5-Adamantan-1-yl-2-methyl-benzoxazol-7-yl)-benzaldehyde.

A mixture of 5-adamantan-1-yl-7-bromo-2-methyl-benzoxazole (0.35 g, 1.01 mmol), 4-formyl-boronic acid (0.16 g, 1.06 mmol) and sodium carbonate (0.32 g, 3.03 mmol) in toluene (14.5 mL), ethanol (3.5 mL) and water (2 mL) was degassed with argon for 40 minutes. Tetrakis(triphenylphosphine)palladium(0) (0.035 g, 0.03 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 9:1) to give 0.30 g of 4-(5-Adamantan-1-yl-2-methyl-benzoxazol-7-yl)-benzaldehyde. ¹H NMR (300 MHz; CDCl₃): δ 1.80 (br s, 6H), 2.02 (2s, 6H), 2.15 (brm, 3H), 2.68 (s, 3H), 7.56 (d, J=1.5 Hz, 1H), 7.68 (d, J=1.5 Hz, 1H), 8.02 (s, 4H), 10.09 (s, 1H).

b. 5-adamantan-1-yl-7-bromo-2-methyl-benzoxazole.

4-Adamantan-1-yl-2-amino-6-bromo-phenol (2.12 g, 6.58 mmol) was dissolved in toluene (20 mL) and acetic anhydride (10 mL). p-Toluene sulfonic acid (1.25 g, 6.58 mmol) and the mixture was heated at reflux for 40 hours. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 9.6:0.4) to give 0.7 g of 5-adamantan-1-yl-7-bromo-2-methyl-benzoxazole. ¹H NMR (300 MHz; CDCl₃): δ 1.77 (br s, 6H), 1.93 (2s, 6H), 2.11 (brm, 3H), 2.65 (s, 3H), 7.45 (d, J=1.8 Hz, 1H), 7.55 (d, J=1.8 Hz, 1H).

c. 4-Adamantan-1-yl-2-amino-6-bromo-phenol.

4-Adamantan-1-yl-2-bromo-6-nitro-phenol (2.83 g, 8.04 mmol) was dissolved in ethanol (100 mL) and SnCl₂-2H₂O (9.07 g, 40.2 mmol) was added and the mixture was heated at reflux under argon for 1 hour. The solution was cooled to room temperature, quenched into ice, neutralized to pH 7 with sodium carbonate and diluted with ethyl acetate. The mixture was filtered through celite and extracted with ethyl acetate. The organic layer was washed successively with water and brine, dried over anhydrous magnesium sulfate, filtered and evaporated to give 2.12 g of 4-Adamantan-1-yl-2-amino-6-bromo-phenol (82%). ¹H NMR (300 MHz; CDCl₃): δ 1.77 (br s, 6H), 1.81 (br s, 6H), 2.06 (br s, 3H), 2.85 (br s, 1H), 6.65 (s, 1H), 6.78 (s, 1H).

d. 4-Adamantan-1-yl-2-bromo-6-nitro-phenol.

4-Adamantan-1-yl-2-bromo-phenol (10 g, 32.6 mmol) was dissolved in dichloromethane (550 mL) and NO₂—BF₄ (0.5 M in sulfolane, 84 mL) was added under argon at 0° C. The reaction mixture was allowed to warm to room temperature and stirred at room temperature for 18 hours. The solvent was evaporated and water was added to the residue to form a gummy precipitate that was collected. The compound was further treated with ethanol and evaporated then dissolved in the minimum amount of hot ethyl acetate then hexane was added. The solution was filtered and evaporated to give 7.71 g of 4-Adamantan-1-yl-2-bromo-6-nitro-phenol (67%). ¹H NMR (300 MHz; CDCl₃): δ 1.77 (br 2s, 6H), 1.85 (br s, 6H), 2.10 (br s, 3H), 7.87 (s, 1H), 8.01 (s, 1H).

e. 4-Adamantan-1-yl-2-bromo-phenol.

2-bromophenol (3.1 mL, 26.5 mmol) and 1-adamantanol (4.05 g, 26.5 mmol) were dissolved in dichloromethane (25 mL) and sulfuric acid (1.5 mL) was added. The reaction mixture was stirred under argon at room temperature overnight. The reaction mixture was poured into water then extracted with dichloromethane. The organic was washed successively with water and brine, dried over anhydrous magnesium sulfate, filtered and evaporated. The residue was purified on silica gel (eluent: hexane:ethyl acetate, 9.5:0.5) to give 7.34 g of 4-Adamantan-1-yl-2-bromo-phenol (90%). ¹H NMR (300 MHz; CDCl₃): δ 1.75 (br s, 6H), 1.85 (br s, 6H), 2.07 (br s, 3H), 5.36 (s, 1H), 6.95 (d, J=8.4 Hz, 1H), 7.20 (dd, J₁=2.1 Hz, J₂=8.4 Hz, 1H), 7.40 (d, J=2.1 Hz, 1H).

### Example 8

### 5-[4-(5-Adamantan-1-yl-2-methyl-benzoxazol-7-yl)-benzylidene]-2-thioxo-thiazolidin-4-one

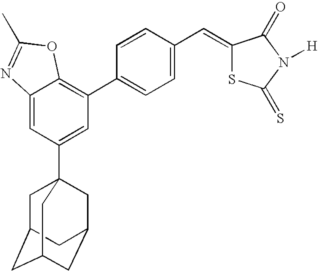

Prepared in a similar manner as described in Example 1 using 4-(5-Adamantan-1-yl-2-methyl-benzoxazol-7-yl)-benzaldehyde (example 7a) and rhodanine. mp>360° C., ¹H NMR (300 MHz; DMSO-d6): δ 1.79 (broad s, 6H), 2.00 (broad d, 6H), 2.12 (br s, 3H), 2.66 (s, 3H), 7.61 (d, J=2.1 Hz, 2H), 7.70 (s, 1H), 7.73 (d, J=8.4 Hz, 2H), 8.06 (d, J=8.4 Hz, 2H), 8.20 (s, 1H).

### Example 9

### 5-[3-(5-Adamantan-1-yl-2-methyl-benzoxazol-7-yl)-benzylidene]-thiazolidine-2,4-dione

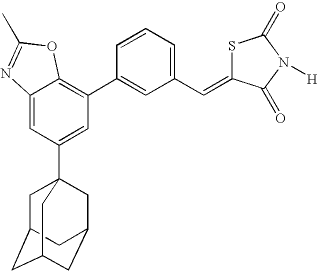

Prepared in a similar manner as described in Example 7 using 3-(5-Adamantan-1-yl-2-methyl-benzoxazol-7-yl)-benzaldehyde. mp>360° C., ¹H NMR (300 MHz; DMSO-d6): δ 1.79 (broad s, 6H), 2.00 (broad d, 6H), 2.12 (br s, 3H), 2.66 (s, 3H), 7.61 (d, J=2.1 Hz, 2H), 7.70 (s, 1H), 7.73 (d, J=8.4 Hz, 2H), 8.06 (d, J=8.4 Hz, 2H), 8.20 (s, 1H).

The intermediate 3-(5-Adamantan-1-yl-2-methyl-benzoxazol-7-yl)-benzaldehyde was prepared as follows:

a. 3-(5-Adamantan-1-yl-2-methyl-benzoxazol-7-yl)-benzaldehyde.

A mixture of 5-adamantan-1-yl-7-bromo-2-methyl-benzoxazole (example 7b) (0.35 g, 1.01 mmol), 3-formyl-boronic acid (0.16 g, 1.06 mmol) and sodium carbonate (0.32 g, 3.03 mmol) in toluene (14.5 mL), ethanol (3.5 mL) and water (2 mL) was degassed with argon for 30 minutes. Tetrakis(triphenylphosphine)palladium(0) (0.035 g, 0.03 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 9:1) to give 0.38 g of 3-(5-Adamantan-1-yl-2-methyl-benzoxazol-7-yl)-benzaldehyde. ¹H NMR (300 MHz; CDCl₃): δ 1.81 (br s, 6H), 2.02 (2s, 6H), 2.15 (br m, 3H), 2.68 (s, 3H), 7.53 (d, J=1.8 Hz, 1H), 7.67 (d, J=1.8 Hz, 1H), 7.70 (d, J=7.5 Hz, 1H), 7.90 (dd, J₁=1.8 Hz, J₂=7.5 Hz, 1H), 8.12 (dd, J₁=1.8 Hz, J₂=7.5 Hz, 1H), 8.32 (d, J=1.8 Hz, 1H), 10.14 (s, 1H).

### Example 10

### 5-[3-(5-Adamantan-1-yl-2-methyl-benzooxazol-7-yl)-benzylidene]-2-thioxo-thiazolidin-4-one

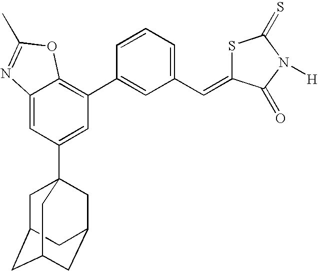

Prepared in a similar manner as described in Example 1 using 3-(5-Adamantan-1-yl-2-methyl-benzoxazol-7-yl)-benzaldehyde (example 9a) and rhodanine. mp 310° C., ¹H NMR (300 MHz; DMSO-d6): δ 1.78 (br s, 6H), 2.00 (br s, 6H), 2.10 (br s, 3H), 2.67 (s, 3H), 7.60-7.75 (m, 4H), 7.79 (s, 1H), 8.04 (d, J=7.5 Hz, 1H), 8.17 (s, 1 H).

### Example 11

### 5-[6-(7-Cyclohexyl-2-methyl-benzoxazol-5-yl)-pyridin-3-ylmethylene]-thiazolidine-2,4-dione

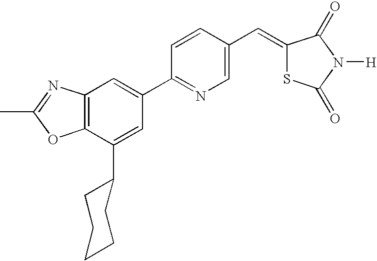

A solution of 5-[6-(3-Amino-5-cyclohexyl-4-hydroxy-phenyl)-pyridin-3-ylmethylene]-thiazolidine-2,4-dione (100 mg, 0.253 mmol) in triethyl ortho acetate (3 mL) was heated at 100° C. for 3.5 hours. The reaction was cooled to 0° C., filtered and washed with hexane. The compound was further purified by precipitation from ethanol and water to give 65 mg of 5-[6-(7-Cyclohexyl-2-methyl-benzoxazol-5-yl)-pyridin-3-ylmethylene]-thiazolidine-2,4-dione. mp 314-316° C., ¹H NMR (300 MHz; DMSO-d6): δ 1.3-1.5 (m, 3H), 1.6-2.0 (m, 7H), 2.65 (s, 3H), 2.98 (m, 1H), 7.84 (s, 1H), 7.98 (dd, J₁=1.8 Hz, J₂=8.7 Hz, 1H), 8.04 (s, 1H), 8.21 (m, 2H), 8.88 (s, 1H), 12.69 (br s, 1H).

The intermediate 5-[6-(3-Amino-5-cyclohexyl-4-hydroxy-phenyl)-pyridin-3-ylmethylene]-thiazolidine-2,4-dione was prepared as follows:

a. 5-[6-(3-Amino-5-cyclohexyl-4-hydroxy-phenyl)-pyridin-3-ylmethylene]-thiazolidine-2,4-dione.

To a solution of 5-[6-(3-Cyclohexyl-4-hydroxy-5-nitro-phenyl)-pyridin-3-ylmethylene]-thiazolidine-2,4-dione (1.80 g, 4.23 mmol) in THF (200 mL) and Ethanol (200 mL) was added aqueous sodium hypophosphite (2.4 M, 8.82 mL, 21.15 mmol) and Pd/C (1 g). The reaction mixture was stirred at room temperature overnight. The catalyst was filtered and washed with THF. The solution was concentrated to a volume of 75 mL and water (150 mL) was added. The compound precipitated and was collected to give 850 mg of 5-[6-(3-Amino-5-cyclohexyl-4-hydroxy-phenyl)-pyridin-3-ylmethylene]-thiazolidine-2,4-dione. ¹H NMR (300 MHz; DMSO-d₆): δ 1.2-1.5 (m, 5H), 1.7-1.9 (m, 5H), 2.95 (br t, 1H), 7.27 (d, J=2.1 Hz, 1H), 7.34 (d, J=2.4 Hz, 1H), 7.82 (s, 1H), 7.92 (d, J=1.2 Hz, 1H), 8.81 (br s, 1H).

### Example 12

### 5-[6-(7-Cyclohexyl-benzoxazol-5-yl)-pyridin-3-ylmethylene]-thiazolidine-2,4-dione

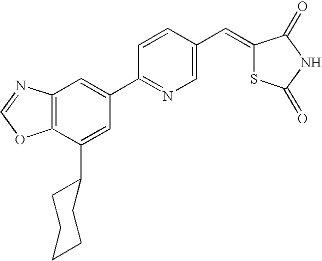

Prepared in a similar manner as described in Example 1 using 5-[6-(3-Amino-5-cyclohexyl-4-hydroxy-phenyl)-pyridin-3-ylmethylene]-thiazolidine-2,4-dione and triethyl orthoformate. mp 282° C. ¹H NMR (300 MHz; DMSO-d₆): δ 1.3-1.5 (m, 3H), 1.6-2.0 (m, 7H), 3.02 (tt, 1H, J=3.0, 3.0, 12.0, 12.0 Hz), 7.86 (s, 1H), 8.00 (dd, 1H, J=2.4, 8.4 Hz), 8.14 (d, 1H, J=1.2 Hz), 8.24 (d, 1H, J=8.7 Hz), 8.36 (d, 1H, J=1.5 Hz), 8.78 (s, 1H), 8.90 (d, 1H, J=2.1 Hz), 12.68 (bs, 1H).

The intermediate 5-[6-(3-Amino-5-cyclohexyl-4-hydroxy-phenyl)-pyridin-3-ylmethylene]-thiazolidine-2,4-dione was synthesized as follows:

a. 5-[6-(3-Amino-5-cyclohexyl-4-hydroxy-phenyl)-pyridin-3-ylmethylene]-thiazolidine-2,4-dione.

To a solution of 5-[6-(3-cyclohexyl-4-hydroxy-5-nitro-phenyl)-pyridin-3-ylmethylene]-thiazolidine-2,4-dione (1.80 g, 4.23 mmol) in tetrahydrofuran/ethanol (1:1, 400 mL) was added an aqueous solution of sodium hypophosphite (5 eq., 1.86 g in 8.8 ml water) and palladium on charcoal (10%, wet, 1.0 g). The mixture was stirred overnight at room temperature. The catalyst was removed by filtration and the eluent was concentrated to about 75 mL. Water was added (150 mL) and the mixture was stirred for 2 hours. The product was filtered off, washed with water and dried to give 0.85 g (51%) of the title compound.

¹H-NMR (300 MHz, DMSO-d₆): δ 1.30-1.50 (m, 5H), 1.70-1.90 (m, 5H), 2.94 (br s, 1H), 7.27 (d, J=2.1 Hz, 1H), 7.34 (d, J=2.4 Hz, 1H), 7.82 (s, 1H), 7.92 (m, 2H), 8.81 (s, 1H).

b. 5-[6-(3-Cyclohexyl-4-hydroxy-5-nitro-phenyl)-pyridin-3-ylmethylene]-thiazolidine-2,4-dione.

To a solution of 6-(3-cyclohexyl-4-hydroxy-5-nitro-phenyl)-pyridine-3-carbaldehyde (2.86 g, 8.76 mmol) in toluene (30 ml) was added piperidine (0.1 eq., 90 μl), acetic acid (0.1 eq., 50 μl), and 2,4-thiazolidinedione (1.2 eq., 1.23 g). The reaction mixture was refluxed overnight using a Dean-Starck apparatus under an argon atmosphere, then cooled to 0° C. and filtered. The precipitate was washed with cold toluene (10 ml) and hexane (10 ml), briefly dried and chromatographed on silica gel (hexane/ethyl acetate 7:3) to afford 2.01 g (54% yield) 5-[6-(3-cyclohexyl-4-hydroxy-5-nitro-phenyl)-pyridin-3-ylmethylene]-thiazolidine-2,4-dione. ¹H-NMR (300 MHz, DMSO-d₆): δ 1.25-1.60 (m, 5H), 1.70-1.90 (m, 5H), 3.05 (br t, J=11.7 Hz, 1H), 7.86 (s, 1H), 8.00 (dd, J₁=2.4 Hz, J₂=8.4 Hz, 1H), 8.23 (d, J=8.7 Hz, 1H), 8.33 (d, J=2.1 Hz, 1H), 8.61 (d, J=2.4 Hz, 1H), 8.90 (d, J=2.4 Hz, 1H), 10.83 (br s), 12.70 (br s).

c. 6-(3-Cyclohexyl-4-hydroxy-5-nitro-phenyl)-pyridine-3-carbaldehyde.

To a solution of 6-(3-cyclohexyl-4-hydroxy-phenyl)-pyridine-3-carbaldehyde (4.10 g, 14.57 mmol) in dichloromethane (100 mL) was added dropwise nitronium tetrafluoroborate (0.5 M solution in sulfolane, 3.5 eq., 102 mL). The mixture was stirred at room temperature for 1 hour after which time it was quenched by the addition of water. The aqueous phase was extracted with dichloromethane and the combined organic phases were dried with sodium sulfate, filtered and evaporated. The crude product was suspended in hot ethanol (100 mL) and stirred for 2 hours. Water was added (150 mL) and the precipitate was filtered and dried to give 2.87 g (60% yield) of 6-(3-cyclohexyl-4-hydroxy-5-nitro-phenyl)-pyridine-3-carbaldehyde. ¹H-NMR (300 MHz, CDCl₃): δ 1.45-1.7 (m, 5H), 1.8-2.0 (m, 5H), 3.15 (br s, 1H), 7.91 (d, J=8.1 Hz, 1H), 8.25 (dd, J₁=2.1 Hz, J₂=8.1 Hz, 1H), 8.34 (d, J=2.4 Hz, 1H), 8.70 (d, J=2.1 HZ, 1H), 9.12 (d, J=2.1 Hz, 1H), 10.15 (s, 1H), 11.27 (s, 1H).

d. 6-(3-Cyclohexyl-4-hydroxy-phenyl)-pyridine-3-carbaldehyde.

To a solution of 6-[4-(tert-butyl-dimethyl-silanyloxy)-3-cyclohexyl-phenyl]-pyridine-3-carbaldehyde (11.03 g, 27.88 mmol) in tetrahydrofuran (200 mL) at 0° C. was added dropwise tetrabutylammonium fluoride (1 M solution in tetrahydrofuran, 1.2 eq., 33.5 ml) and stirred for 2 hours. The reaction was quenched by addition of water (50 mL) and brine (20 mL). The layers were separated and the aqueous layer was extracted with ethyl acetate. The combined organic phases were dried with sodium sulfate, filtered, evaporated and chromatographed on silica gel (hexane/ethyl acetate 8:2, then 6:4) to give 4.39 g, (56% yield) of 6-(3-cyclohexyl-4-hydroxy-phenyl)-pyridine-3-carbaldehyde. ¹H-NMR (300 MHz, CDCl₃): δ 1.4-1.6 (m, 5H), 1.7-2.0 (m, 5H), 2.88 (br t, 1H), 6.85 (d, J=8.7 Hz, 1H), 7.78 (dd, J₁=2.4 Hz, J₂=8.4 Hz, 1H), 7.83 (d, J=7.8 Hz, 1H), 7.98 (d, J=2.1 Hz, 1H), 8.18 (dd, J₁=2.1 Hz, J₂=8.4 Hz, 1H), 9.07 (d, J=2.1 Hz, 1H), 10.10 (s, 1H).

e. 6-[4-(tert-Butyl-dimethyl-silanyloxy)-3-cyclohexyl-phenyl]-pyridine-3-carbaldehyde.

A mixture of 4-(tert-butyl-dimethyl-silanyloxy)-3-cyclohexyl-phenyl-boronic acid (14.30 g, 42.80 mmol), 6-bromo-pyridine-3-carbaldehyde (1.2 eq., 8.73 g), potassium carbonate (3 eq., 11.6 g) in toluene/ethanol/water (8:2:1; 165 mL) was degassed with argon. Palladium tetrakis(triphenylphosphine) (0.05 eq., 2.32 g) was added and the reaction was set to reflux overnight. Water was added and the mixture was extracted with ethyl acetate three times. The combined organic layers were dried with sodium sulfate, filtered and evaporated. The crude product was subjected to silica gel chromatography (hexane/ethyl acetate 95:5, then 9:1) to yield 11.03 g (65%) of the title compound. ¹H-NMR (300 MHz, CDCl₃): δ 0.29 (s, 6H), 1.05 (s, 9H), 1.2-1.5 (m, 5H), 1.7-1.95 (m, 5H), 2.99 (br s, 1H), 6.89 (d, J=8.4 Hz, 1H), 7.79 (dd, J₁=2.4 Hz, J₂=8.4 Hz, 1H), 7.83 (d, J=8.4 Hz, 1H), 7.97 (d, J=2.4 Hz, 1H), 8.17 (dd, J=2.1 Hz, J₂=8.1 Hz, 1H), 9.07 (d, J=2.4 Hz, 1H), 10.10 (s, 1H).

f. 4-(tert-Butyl-dimethyl-silanyloxy)-3-cyclohexyl-phenyl-boronic acid.

(4-Bromo-2-cyclohexyl-phenoxy)-tert-butyl-dimethyl-silane (14.78 g, 40.00 mmol) was dissolved in anhydrous tetrahydrofuran (200 mL) and cooled to −78° C. n-Butyllithium (2.5 M solution in hexane, 1.5 eq., 24 mL) was added dropwise followed by the dropwise addition of triisopropyl borate (3 eq., 28 mL). The resulting solution was allowed to warm up to room temperature while stirring overnight. The reaction was quenched by the addition of saturated aqueous ammonium chloride solution (200 mL). Water was added until the white precipitate dissolved and the product was extracted with ethyl acetate. The combined organic phases were dried with sodium sulfate, filtered, dried and stirred in hexane. The product was filtered and dried to give 14.3 g of 4-(tert-Butyl-dimethyl-silanyloxy)-3-cyclohexyl-phenyl-boronic acid. ¹H-NMR (300 MHz, DMSO-d₆/D₂O): δ 1.2-1.45 (m, 5H), 1.65-1.85 (m, 5H), 2.87 (br t, 1H), 6.74 (d, J=8.1 Hz, 1H), 7.50 (dd, J₁=1.8 Hz, J₂=8.1 Hz, 1H), 7.65 (d, J=1.5 Hz, 1H).

g. (4-Bromo-2-cyclohexyl-phenoxy)-tert-butyl-dimethyl-silane.

A solution of 4-bromo-2-cyclohexyl-phenol (31.86 g, 0.125 mol), triethylamine (1.5 eq., 25.9 mL) and tert.-butyl-dimethyl-silyl chloride (1.3 eq., 24.76 g) in dichloromethane (200 mL) was stirred overnight at room temperature. The reaction was quenched with water (30 ml), the organic layer was separated and the aqueous layer was extracted with dichloromethane. The combined organic phases were dried with sodium sulfate, filtered and evaporated. Silica gel chromatography (100% hexane) yielded (4-bromo-2-cyclohexyl-phenoxy)-tert-butyl-dimethyl-silane (38.24 g, 83%). ¹H-NMR (300 MHz, CDCl₃): δ 1.2-1.45 (m, 5H), 1.7-1.9 (m, 5H), 2.88 (br t, J=11.1 Hz, 1H), 6.63 (d, J=8.4 Hz, 1H), 7.12 (dd, J₁=2.7 Hz, J₂=8.4 Hz, 1H), 7.25 (d, 1H).

h. 4-Bromo-2-cyclohexyl-phenol.

A solution of 2-cyclohexyl-phenol (20.52 g, 0.116 mol) and pyridinium tribromide (1.05 eq., 43.44 g) in dichloromethane (250 mL) was stirred for 30 min at room temperature. The reaction was quenched by the addition of water. The aqueous phase was extracted with dichloromethane. The combined organic phases were dried with sodium sulfate, filtered and evaporated to give 32.03 g of 4-bromo-2-cyclohexyl-phenol. ¹H-NMR (300 MHz, CDCl₃): δ 1.2-1.5 (m, 5H), 1.7-1.9 (m, 5H), 2.79 (br t, 1H), 4.81 (br s, 1H), 6.64 (d, J=8.7 Hz, 1H), 7.14 (dd, J₁=2.7 Hz, J₂=8.1 Hz, 1H), 7.26 (m, 1H).

### Example 13

### 5-[6-(7-Cyclohexyl-2-trichloromethyl-benzoxazol-5-yl)-pyridin-3-ylmethylene]-thiazolidine-2,4-dione

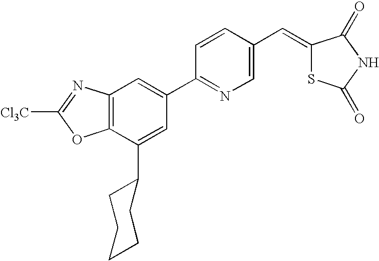

Prepared in a similar manner as described in Example 11 using 5-[6-(3-Amino-5-cyclohexyl-4-hydroxy-phenyl)-pyridin-3-ylmethylene]-thiazolidine-2,4-dione and methyl 2,2,2-trichloroacetimidate. mp 253° C. ¹H NMR (300 MHz; DMSO-d₆): δ 1.25-1.55 (m, 3H), 1.6-1.9 (m, 7H), 3.06 (tt, 1 H, J=3.6, 11.7 Hz), 7.88 (s, 1H), 8.04 (dd, 1H, J=2.4, 8.4 Hz), 8.28 (d, 1H, J=8.7 Hz), 8.30 (d, 1H, J=1.8 Hz), 8.51 (d, 1 H, J=1.2 Hz), 8.94 (d, 1H, J=2.4 Hz), 12.73 (bs, 1H).

### Example 14

### 5-[6-(7-Adamantan-1-yl-2-amino-benzoxazol-5-yl)-pyridin-3-ylmethylene]-thiazolidine-2,4-dione

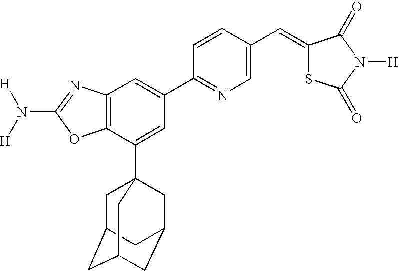

A 5 M solution of cyanogen bromide in acetonitrile (0.57 ml, 2.85 mmol, 2.5 eq.) was added to a suspension of 5-[6-(3-Amino-5-cyclohexyl-4-hydroxy-phenyl)-pyridin-3-ylmethylene]-thiazolidine-2,4-dione (example 11) (500 mg, 1.12 mmol) in anhydrous ethanol (30 mL) and stirred for 5 days at ambient temperature. The mixture was concentrated to approximately 10 ml. The precipitate was filtered, washed with ethanol/water 1:1, then water and dried. Yield: 340 mg, 64%. mp>360° C. ¹H NMR (300 MHz; DMSO-d₆): δ 1.80 (br s, 6H), 2.12 (br s, 9H), 7.62 (br s, 2H), 7.77 (d, 1H, J=1.5 Hz), 7.82 (d, 1H, J=1.5 Hz), 7.87 (s, 1H), 7.98 (dd, 1H, J=2.4, 8.7 Hz), 8.15 (d, 1H, J=8.7 Hz), 8.89 (d, 1H, J=2.1 Hz), 12.70 (br s, 1H).

### Example 15

### 5-{6-[7-(1,1-Dimethyl-propyl)-benzoxazol-5-yl]-pyridin-3-ylmethylene}-thiazolidine-2,4-dione

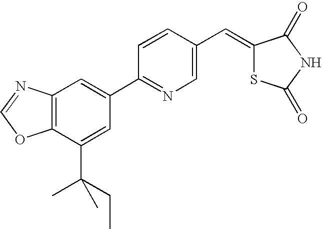

Prepared in a similar manner as described in Example 12 using 5-{6-[3-amino-5-(1,1 dimethyl-propyl)-4-hydroxy-phenyl]-pyridin-3-ylmethylene}-thiazolidine-2,4-dione and triethyl orthoformate. Yield: 104 mg, 51%. mp 259° C. ¹H NMR (300 MHz; DMSO-d₆): δ 0.64 (t, 3H, J=7.8 Hz), 1.48 (s, 6H), 1.91 (q, 2H, J=7.5 Hz), 7.889 (s, 1H), 8.042 (dd, 1H, J=8.4, 2.4 Hz), 8.147 (d, 1H, J=1.5 Hz), 8.278 (d, 1H, J=8.7 Hz), 8.415 (d, 1H, J=1.8 Hz), 8.819 (s, 1H), 8.948 (d, 1H, J=2.4 Hz), 12.752 (bs, 1H).

The intermediate 5-{6-[3-Amino-5-(1,1-dimethyl-propyl)-4-hydroxy-phenyl]-pyridin-3-ylmethylene}-thiazolidine-2,4-dione was prepared as follows:

a. 5-{6-[3-Amino-5-(1,1 dimethyl-propyl)-4-hydroxy-phenyl]-pyridin-3-ylmethylene}-thiazolidine-2,4-dione.

To a solution of 5-{6-[3-(1,1-dimethyl-propyl)-4-hydroxy-5-nitro-phenyl]-pyridin-3-ylmethylene}-thiazolidine-2,4-dione (5.314 g, 13.9 mmol) in tetrahydrofuran/ethanol (2:1, 900 ml) was added an aqueous solution of sodium hypophosphite (6 eq., 7.30 g in 40 ml water) and palladium on charcoal (10%, wet, 2.0 g). The mixture was refluxed for 4 hours. The palladium was removed by filtration and the eluent was concentrated to about 20 mL. Ethanol was added (500 mL) followed by water (500 mL) and the crude product was obtained by filtration. Pure product was obtained by preparative HPLC (YMC-Pack, ODS-A, AA 12S21-2551DR, S-15/30, 12 nm, NO. 50256809(D); isocratic elution with 50% (water/0.02% TFA)/50% acetonitrile) to give 1.42 g (27%) of the title compound. 1H-NMR (300 MHz, DMSO-d₆): δ 0.62 (t, J=7.2 Hz, 3H), 1.35 (s, 6H), 1.89 (q, J=7.8 Hz, 2H), 7.30 (d, J=2.4 Hz, 1H), 7.40 (d, J=2.1 Hz, 1H), 7.81 (d, J=3.9 Hz, 1H), 7.88 (d, J=8.4 Hz, 1H), 7.93 (dd, J₁=2.4 Hz, J₂=8.7 Hz, 1H), 8.82 (d, J=2.1 Hz, 1H).

b. 5-{6-[3-(1,1-dimethyl-propyl)-4-hydroxy-5-nitro-phenyl]-pyridin-3-ylmethylene}-thiazolidine-2,4-dione.

To a solution of 5-{6-[3-(1,1-dimethyl-propyl)-4-hydroxy-phenyl]-pyridin-3-ylmethylene}-thiazolidine-2,4-dione (6.94 g, 18.8 mmol) in trifluoroacetic acid at 0° C. was added potassium nitrate (1.05 eq., 2.10 g). The solution was stirred at 0° C. for 30 min. and then poured into ice/water. The precipitate was filtered, washed with water until pH=5 and dried briefly to give the title compound used as this in the next step. ¹H-NMR (300 MHz, DMSO-d₆): δ 0.64 (t, J=7.2 Hz, 3H), 1.44 (s, 6H), 1.94 (q, J=7.5 Hz, 2H), 7.88 (s, 1H), 8.03 (dd, J₁=2.4 Hz, J₂=8.4 Hz, 1H), 8.22 (d, J=8.4 Hz, 1H), 8.33 (d, J=1.8 Hz, 1H), 8.66 (d, J=2.1 Hz, 1H), 8.93 (d, J=2.1 Hz, 1H), 11.14 (s, 1H), 12.74 (br s, 1H).

c. 5-{6-[3-(1,1-dimethyl-propyl)-4-hydroxy-phenyl]-pyridin-3-ylmethylene}-thiazolidine-2,4-dione.

To a solution of 6-[3-(1,1-dimethyl-propyl)-4-hydroxy-phenyl]-pyridine-3-carbaldehyde (6.05 g, 22.5 mmol) in toluene (65 mL) was added piperidine (0.05 eq., 111 μl), acetic acid (0.09 eq., 111 μl), and 2,4-thiazolidinedione (1.2 eq., 3.16 g). The reaction mixture was refluxed overnight under an argon atmosphere, then cooled to 0° C. and filtered. The precipitate was washed with cold toluene (10 mL) and hexane (10 mL) and dried to afford 7.11 g (86% yield) of 5-{6-[3-(1,1-dimethyl-propyl)-4-hydroxy-phenyl]-pyridin-3-ylmethylene}-thiazolidine-2,4-dione. ¹H-NMR (300 MHz, DMSO-d₆): δ 0.61 (t, J=7.2 Hz, 3H), 1.36 (s, 6H), 1.87 (q, J=7.5 Hz, 2H), 6.89 (d, J=8.7 Hz, 1H), 7.81-7.84 (m, 2H), 7.94 (dd, J₁=2.4 Hz, J₂=8.7 Hz, 1H), 8.00 (d, J=8.7 Hz, 1H), 8.84 (s, 1H), 9.86 (s, 1H), 12.67 (br s, 1H).
d. 6-[3-(1,1-Dimethyl-propyl)-4-hydroxy-phenyl]-pyridine-3-carbaldehyde.

To a solution of 6-[4-(tert-butyl-dimethyl-silanyloxy)-3-(1,1-dimethyl-propyl)-phenyl]-pyridine-3-carbaldehyde (8.684 g, 22.6 mmol) in tetrahydrofuran at 0° C. was added dropwise tetrabutylammonium fluoride (1 M solution in tetrahydrofuran, 1.2 eq., 27.1 mL) and the mixture stirred for 2 hours. The reaction was quenched by addition of water (50 mL) and brine (20 mL). The layers were separated and the aqueous layer was extracted with ethyl acetate. The combined organic phases were dried with sodium sulfate, filtered and evaporated to give 6.05 g, (99% yield) of 6-[3-(1,1-dimethyl-propyl)-4-hydroxy-phenyl]-pyridine-3-carbaldehyde. ¹H-NMR (300 MHz, CDCl₃): δ 0.70 (t, J=7.5 Hz, 3H), 1.45 (s, 6H), 1.92 (q, J=7.5 Hz, 2H), 6.77 (d, J=8.4 Hz, 1H), 7.82 (d, J=8.1 Hz, 2H), 8.01 (d, J=2.1 Hz, 1H), 8.18 (dd, J₁=2.1 Hz, J₂=8.1 Hz, 1H), 9.07 (d, J=2.1 Hz, 1H), 10.10 (s, 1H).

e. 6-[4-(tert-Butyl-dimethyl-silanyloxy)-3-(1,1-dimethyl-propyl)-phenyl]-pyridine-3-carbaldehyde.

A mixture of 4-(tert-butyl-dimethyl-silanyloxy)-3-(1,1-dimethyl-propyl)-phenyl-boronic acid (10.00 g, 31.0 mmol), 6-bromo-pyridine-3-carbaldehyde (1 eq., 5.77 g), potassium carbonate (3 eq., 12.85 g) in toluene/ethanol/water (8:2:1; 300 mL) was degassed with argon. Palladium tetrakis(triphenylphosphine) (0.05 eq., 1.79 g) was added and the reaction was set to reflux overnight. Water was added and the mixture was extracted with ethyl acetate three times. The combined organic layers were dried with sodium sulfate, filtered and evaporated. The crude product was subjected to silica gel chromatography (hexane/ethyl acetate 85:15) to yield 8.74 g (74%) of the title compound. ¹H-NMR (300 MHz, CDCl₃): δ 0.36 (s, 6H), 0.68 (t, J=7.5 Hz, 3H), 1.05 (s, 9H), 1.42 (s, 6H), 1.91 (q, J=7.5 Hz, 2H), 6.91 (d, J=8.4 Hz, 1H), 7.80-7.84 (m, 2H), 8.02 (d, J=2.4 Hz, 1H), 8.17 (dd, J₁=2.1 Hz, J₂=8.1 Hz, 1H), 9.07 (d, J=2.1 Hz, 1H), 10.10 (s, 1H).

f. 4-(tert-Butyl-dimethyl-silanyloxy)-3-(1,1-dimethyl-propyl)-phenyl-boronic acid.

[4-Bromo-2-(1,1-dimethyl-propyl)-phenoxy]-tert-butyl-dimethyl-silane (25.66 g, 71.8 mmol) was dissolved in tetrahydrofuran (200 mL) and cooled to −78° C. n-Butyllithium (2.5 M solution in hexane, 1.5 eq., 43.1 mL) was added dropwise followed by the dropwise addition of triisopropyl borate (3 eq., 50 mL). The resulting solution was allowed to warm up to room temperature while stirring overnight. The reaction was quenched by the addition of saturated aqueous ammonium chloride solution (200 mL). Water was added until the white precipitate dissolved and the product was extracted with ethyl acetate. The combined organic phases were dried with sodium sulfate, filtered, dried and subjected to silica gel chromatography (hexane/ethyl acetate 7:3, then 100% ethyl acetate) to give 19.24 g (83%) of 3-4-(tert-butyl-dimethyl-silanyloxy)-3-(1,1-dimethyl-propyl)-phenyl-boronic acid.

g. [4-Bromo-2-(1,1-dimethyl-propyl)-phenoxy]-tert-butyl-dimethyl-silane.

A solution of 4-bromo-2-(1,1-dimethyl-propyl)-phenol (20.765 g, 85.4 mmol), triethylamine (1.5 eq., 17.9 mL), 4-(dimethylamino)-pyridine (0.03 eq., 213 mg) and tert.-butyl-dimethyl-silyl chloride (1.1 eq., 14.16 g) in dichloromethane (200 mL) was stirred for 3 days at room temperature. The reaction was quenched with water (30 mL), the organic layer was separated and the aqueous layer was extracted with dichloromethane. The combined organic phases were dried with sodium sulfate, filtered and evaporated. Silica gel chromatography (100% hexane) yielded [4-bromo-2-(1,1-dimethyl-propyl)-phenoxy]-tert-butyl-dimethyl-silane (25.66 g, 89%). ¹H-NMR (300 MHz, CDCl₃): δ 0.30 (s, 6H), 0.63 (t, J=7.5 Hz, 3H), 1.01 (s, 9H), 1.30 (s, 6H), 1.83 (q, J=7.5 Hz, 2H), 6.65 (d, J=8.4 Hz, 1H), 7.15 (dd, J=2.7 Hz, J₂=8.7 Hz, 1H), 7.29 (d, J=2.4 Hz, 1H).

### Example 16

### 5-{6-[7-(1,1-Dimethyl-propyl)-2-methyl-benzooxazol-5-yl]-pyridin-3-ylmethylene}-thiazolidine-2,4-dione

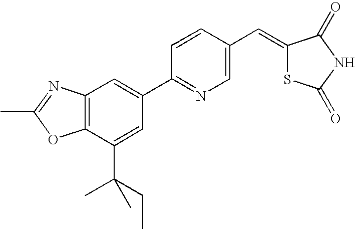

A solution of 5-{6-[3-amino-5-(1,1 dimethyl-propyl)-4-hydroxy-phenyl]-pyridin-3-ylmethylene}-thiazolidine-2,4-dione (example 15a) (170 mg, 0.443 mmol) in triethyl orthoacetate (4 ml) was stirred at 100° C. for 5 hours. The mixture was cooled to 0° C. and filtered. The precipitate was washed with hexane and briefly dried. The product was purified by precipitation from ethanol with water. Yield: 98 mg, 54%. mp 312° C. ¹H NMR (300 MHz; DMSO-d₆): δ 0.65 (t, 3H, J=7.2 Hz), 1.467 (s, 6H), 1.89 (q, 2H, J=7.2 Hz), 2.66 (s, 3H), 7.87 (s, 1H), 8.01 (d, 1H, J=2.4 Hz), 8.04 (s, 1H), 8.23-8.27 (m, 2H), 8.93 (d, 1H, J=2.1 Hz), 12.70 (bs, 1H).

### Example 17

### N-{7-Adamantan-1-yl-5-[5-(2,4-dioxo-thiazolidin-5-ylidenemethyl)-pyridin-2-yl]-benzooxazol-2-yl}-2,2,2-trifluoro-acetamide.

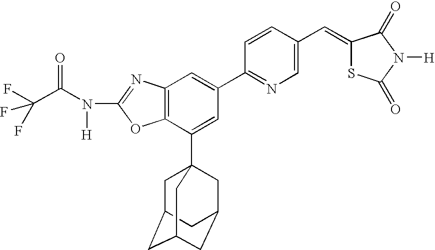

A suspension of 5-[6-(7-adamantan-1-yl-2-amino-benzoxazol-5-yl)-pyridin-3-ylmethylene]-thiazolidine-2,4-dione (example 14) (93 mg, 0.197 mmol), pyridine (8 eq., 128 μl) and trifluoroacetic anhydride (3 eq., 85 μl) in anhydrous tetrahydrofurane (5 mL) was stirred overnight at room temperature. The mixture was separated between water and ethyl acetate, the aqueous phase was extracted three times with ethyl acetate, and all combined organic phases were dried with sodium sulfate, filtered and evaporated. The crude product was refluxed in dichloromethane for one hour and precipitated by addition of hexane. The precipitate was filtered and dried, then refluxed in ethanol for one hour and precipitated by addition of water. The product was collected by filtration and dried to give 62 mg (55%) of the title compound. mp 353° C. ¹H NMR (300 MHz; DMSO-d₆): δ 1.79 (br s, 6H), 2.11 (br s, 9H), 7.85 (s, 1H), 7.97 (d, 1H, J=1.5 Hz), 8.01 (dd, 1H, J=2.4, 8.7 Hz), 8.05 (d, 1H, J=1.5 Hz), 8.13 (d, 1H, J=8.1 Hz), 8.90 (d, 1H, J=2.4 Hz), 12.71 (br s).

### Example 18

### N-{7-Adamantan-1-yl-5-[5-(2,4-dioxo-thiazolidin-5-ylidenemethyl)-pyridin-2-yl]-benzooxazol-2-yl}-acetamide.

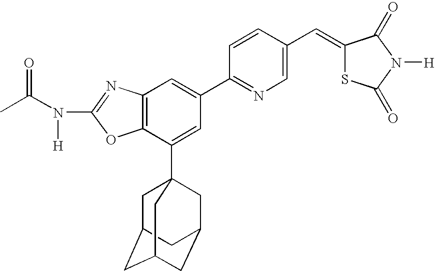

A suspension of 5-[6-(7-adamantan-1-yl-2-amino-benzoxazol-5-yl)-pyridin-3-ylmethylene]-thiazolidine-2,4-dione (example 14) (88 mg, 0.186 mmol), pyridine (12 eq., 180 μL), 4-(dimethylamino)pyridine (1 eq., 22 mg) and acetic anhydride (3.6 eq., 63 μL) in anhydrous tetrahydrofurane (5 mL) was stirred overnight at room temperature. Crude product was obtained by addition of hexane and filtration of the precipitate. Purification was achieved by preparative HPLC (YMC-Pack, ODS-A, AA 12S21-2551DR, S-15/30, 12 nm, NO. 50256809(D); isocratic elution with 45%(water/0.02% TFA)/55% acetonitrile) to give 10 mg (10%) of the title compound. mp>360° C. ¹H NMR (300 MHz; DMSO-d₆): δ 1.79 (s, 6H), 2.10-2.13 (m, 9H), 2.22 (s, 3H), 7.84 (s, 1H), 7.97-8.01 (m, 2H), 8.14 (d, J=1.2 Hz, 1H), 8.20 (d, J=8.4 Hz, 1H), 8.89 (d, J=2.1 Hz, 1H), 11.67 (s, 1H), 12.76 (br s, 1H).

### Example 19

### 5-[6-(7-Benzyloxy-benzoxazol-5-yl)-pyridin-3-ylmethylene]-thiazolidine-2,4-dione

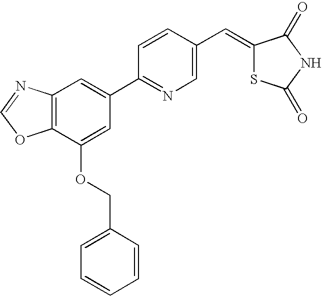

A suspension of 5-[6-(3-Amino-5-benzyloxy-4-hydroxy-phenyl)-pyridin-3-ylmethylene]-thiazolidine-2,4-dione (180 mg, 0.429 mmol) in triethyl orthoformate (3 ml) was stirred at 100° C. for 6 hours. The mixture was cooled to 0° C. and the precipitate was filtered, washed with hexane and dried. The product was purified by preparative high performance liquid chromatography (45% A/55% B; A: water, 0.02% TFA; B: acetonitrile). mp 280° C. ¹H-NMR (300 MHz; DMSO-d₆): δ 5.43 (s, 2H), 7.35-7.44 (m, 3H), 7.53-7.55 (m, 2H), 7.87 (s, 1H), 8.00-8.04 (m, 2H), 8.17 (s, 1H), 8.27 (d, 1H, J=8.1 Hz), 8.78 (s, 1H), 8.92 (d, 1H, J=2.1 Hz), 12.72 (br s, 1H).

The intermediate 5-[6-(3-Amino-5-benzyloxy-4-hydroxy-phenyl)-pyridin-3-ylmethylene]-thiazolidine-2,4-dione was prepared as follows:

a. 5-[6-(3-Amino-5-benzyloxy-4-hydroxy-phenyl)-pyridin-3-ylmethylene]-thiazolidine-2,4-dione.

5-[6-(3-Benzyloxy-4-hydroxy-5-nitro-phenyl)-pyridin-3-ylmethylene]-thiazolidine-2,4-dione (1.44 g, 3.22 mmol) was dissolved in tetrahydrofuran/ethanol (2:1, 600 mL). 8 mL of a 2.4 M aqueous solution of sodium hypophosphite (6 eq., 19.3 mmol) were added followed by palladium on carbon (10%, wet, 1 g). The mixture was refluxed for 6 hours. The catalyst was removed by filtration. The remaining liquid was concentrated and cooled to 0° C. The precipitate was filtered, washed and dried to give 945 mg (70%) of 5-[6-(3-Amino-5-benzyloxy-4-hydroxy-phenyl)-pyridin-3-ylmethylene]-thiazolidine-2,4-dione. ¹H-NMR (300 MHz, DMSO-d₆): δ 5.22 (s, 2H), 7.19 (m, 2H), 7.28-7.41 (m, 3H), 7.55 (m, 2H), 7.83 (s, 1H), 7.92 (m, 2H), 8.80 (s, 1H).

b. 5-[6-(3-Benzyloxy-4-hydroxy-5-nitro-phenyl)-pyridin-3-ylmethylene]-thiazolidine-2,4-dione.

5-[6-(3-Benzyloxy-4-hydroxy-phenyl)-pyridin-3-ylmethylene]-thiazolidine-2,4-dione (1.51 g, 3.733 mmol) was dissolved in trifluoroacetic acid (20 mL) and cooled to 0° C. Potassium nitrate (1.05 eq., 396 mg) was added and stirring was continued for 45 min. The reaction mixture was poured into ice/water. The precipitate was filtered, washed with water until pH=5, and dried to give 5-[6-(3-Benzyloxy-4-hydroxy-5-nitro-phenyl)-pyridin-3-ylmethylene]-thiazolidine-2,4-dione (1.57 g, 94%). ¹H-NMR (300 MHz, DMSO-d₆): δ 5.37 (s, 2H), 7.32-7.44 (m, 3H), 7.55-7.57 (m, 2H), 7.86 (s, 1H), 8.01 (dd, J₁=2.1 Hz, J₂=8.7 Hz, 1H), 8.11 (d, J=2.1 Hz, 1H), 8.19 (d, J=9.0 Hz, 1H), 8.28 (d, J=1.5 Hz, 1H), 8.89 (d, J=2.1 Hz, 1H), 10.87 (br s, 1H), 12.74 (br s, 1H).

c. 5-[6-(3-Benzyloxy-4-hydroxy-phenyl)-pyridin-3-ylmethylene]-thiazolidine-2,4-dione.

To a solution of 6-(3-Benzyloxy-4-hydroxy-phenyl)-pyridine-3-carbaldehyde (1.54 g, 5.06 mmol) in toluene (15 mL) was added piperidine (0.05 eq., 25 μL), acetic acid (0.09 eq., 25 μL), and 2,4-thiazolidinedione (1.2 eq., 711 mg). The reaction mixture was refluxed overnight under an argon atmosphere, then cooled to 0° C. and filtered. The precipitate was washed with cold toluene (5 ml) and hexane (6 ml) and dried to yield 5-[6-(3-Benzyloxy-4-hydroxy-phenyl)-pyridin-3-ylmethylene]-thiazolidine-2,4-dione (1.87 g, 91%). ¹H-NMR (300 MHz, DMSO-d₆): δ 5.22 (s, 2H), 6.94 (d, J=8.7 Hz, 1H), 7.30-7.43 (m, 3H), 7.53 (m, 2H), 7.65 (dd, J₁=1.8 Hz, J₂=8.4 Hz, 1H), 7.85 (m, 2H), 7.95 (dd, J₁=2.1 Hz, J₂=8.4 Hz, 1H), 8.05 (d, J=8.4 Hz, 1H), 8.84 (d, J=1.8 Hz, 1H), 9.64 (s, 1H), 12.69 (br s, 1H).

d. 6-(3-Benzyloxy-4-hydroxy-phenyl)-pyridine-3-carbaldehyde.

A solution of 6-(3-Benzyloxy-4-(tert.-butyl-dimethyl-silanyloxy)-phenyl)-pyridine-3-carbaldehyde (2.25 g, 5.38 mmol) in tetrahydrofuran (65 mL) was cooled to 0° C. A 1 M solution of tetrabutylammonium fluoride in tetrahydrofuran (1.2 eq., 6.46 mL) was added dropwise. After completed addition the solution was stirred for 1.5 hours after which the mixture was separated between water and ethyl acetate. The aqueous phase was extracted and the combined organic layers were dried with sodium sulfate, filtered and evaporated. The crude product was subjected to silica gel chromatography (hexane/ethyl acetate 7:3, then 1:1). Yield: 1.54 g, 94%. ¹H-NMR (300 MHz, CDCl₃): δ 5.25 (s, 2H), 5.94 (s, 1H), 7.05 (d, J=8.7 Hz, 1H), 7.39-7.49 (m, 5H), 7.60 (dd, J₁=1.8 Hz, J₂=8.4 Hz, 1H), 7.83 (d, J=8.4 Hz, 1H), 7.90 (d, J=2.1 Hz, 1H), 8.18 (dd, J₁=1.8 Hz, J₂=8.7 Hz, 1H), 9.05 (d, J=2.1 Hz, 1H), 10.11 (s, 1H).

e. 6-(3-Benzyloxy-4-(tert.-butyl-dimethyl-silanyloxy)-phenyl)-pyridine-3-carbaldehyde.

A mixture of 3-Benzyloxy-4-(tert.-butyl-dimethyl-silanyloxy)-phenyl-boronic acid (2.74 g, 7.66 mmol), 6-Bromo-pyridine-3-carbaldehyde (1 eq., 1.42 g), potassium carbonate (3 eq., 3.18 g) in toluene/ethanol/water (8:2:1; 80 ml) was degassed with argon. Palladium tetrakis(triphenylphosphine) (0.05 eq., 443 mg) was added and the reaction was set to reflux overnight. Water was added and the mixture was extracted with ethyl acetate three times. The combined organic layers were dried with sodium sulfate, filtered and evaporated. The crude product was subjected to silica gel chromatography (hexane/ethyl acetate 85:15) to yield 2.26 g (70%) of 6-(3-Benzyloxy-4-(tert.-butyl-dimethyl-silanyloxy)-phenyl)-pyridine-3-carbaldehyde. ¹H-NMR (300 MHz, CDCl₃): δ 0.13 (s, 6H), 0.98 (s, 9H), 5.18 (s, 2H), 6.99 (d, J=8.1 Hz, 1H), 7.35-7.40 (m, 3H), 7.49 (d, J=6.3 Hz, 2H), 7.56 (dd, J₁=2.1 Hz, J₂=8.4 Hz, 1H), 7.82 (m, 2H), 8.18 (dd, J=1.8 Hz, J₂=8.4 Hz, 1H), 9.07 (d, J=1.2 Hz, 1H), 10.11 (s, 1H).

f. (2-Benzyloxy-4-bromo-phenoxy)-tert.-butyl-dimethyl-silane.

A solution of 2-Benzyloxy-4-bromo-phenol (7.91 g, 28.3 mmol), triethylamine (1.5 eq., 5.9 ml), and tert.-butyl-dimethyl-silyl chloride (1.1 eq., 4.70 g) in dichloromethane (150 ml) was stirred overnight at room temperature. The reaction was quenched with water (30 ml), the organic layer was separated and the aqueous layer was extracted with dichloromethane. The combined organic phases were dried with sodium sulfate, filtered and evaporated. Silica gel chromatography (hexane/ethyl acetate 97:3) yielded pure (2-benzyloxy-4-bromo-phenoxy)-tert.-butyl-dimethyl-silane (5.35 g, 48%). ¹H-NMR (300 MHz, CDCl₃): δ 0.10 (s, 6H), 0.96 (s, 9H), 5.01 (s, 2H), 6.73 (d, J=8.4 Hz, 1H), 6.95 (dd, J₁=2.4 Hz, J₂=8.4 Hz, 1H), 7.03 (d, J=2.1 Hz, 1H), 7.33-7.44 (m, 5H).

g. 2-Benzyloxy-4-bromo-phenol.

2-Benzyloxy-phenol (10.0 g, 49.9 mmol) and pyridinium tribromide (1 eq., 16.0 g) were dissolved in dichloromethane (200 ml) and stirred at room temperature under argon for 1 hour. Water was added, the layers separated and the aqueous layer was extracted twice with dichloromethane. The combined organic phases were dried with sodium sulfate, filtered and evaporated. Silica gel chromatography (hexane/ethyl acetate 8:2) yielded pure 2-benzyloxy-4-bromo-phenol (7.91 g, 57%). ¹H-NMR (300 MHz, CDCl₃): δ 5.08 (s, 2H), 6.83 (d, J=8.1 Hz, 1H), 7.02 (dd, J₁=2.4 Hz, J₂=8.7 Hz, 1H), 7.07 (d, J=2.1 Hz, 1H), 7.30-7.44 (m, 5H).

### Example 20

### 5-[6-(7-Benzyloxy-2-methyl-benzoxazol-5-yl)-pyridin-3-ylmethylene]-thiazolidine-2,4-dione

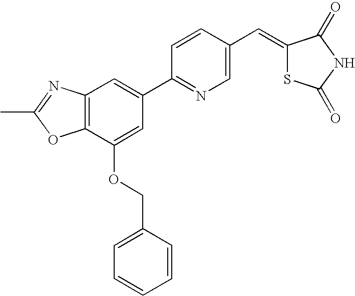

Prepared in a similar manner as described in example 19 using 5-[6-(3-Amino-5-benzyloxy-4-hydroxy-phenyl)-pyridin-3-ylmethylene]-thiazolidine-2,4-dione (180 mg, 0.429 mmol) in triethyl orthoacetate (3 mL). mp 245° C. ¹H-NMR (300 MHz; DMSO-d₆): δ 2.64 (s, 3H), 5.42 (s, 2H), 7.35-7.47 (m, 3H), 7.55 (d, J=6.9 Hz, 2H), 7.88 (s, 1H), 7.94 (s, 1H), 8.02 (dd, J₁=1.8 Hz, J₂=9.0 Hz, 1H), 8.05 (s, 1H), 8.25 (d, J=8.4 Hz, 1H), 8.92 (d, J=1.5 Hz, 1H), 12.71 (br s).

### Example 21

### In vitro Testing of Cancer Drug Candidates, Human Cancer Cell Based Assays

Representative compounds of the invention were screened for anti-cancer activity by the procedures and methods described below. The following human cancer cell lines were used to detect anti-cancer activity.
The breast cancer cell line MDA-MB468 served to detect anti-breast cancer activity.
The prostate cancer cell line PC-3 was used to detect anti-prostate cancer activity
The non-small-cell lung cancer cell line A549 was used to detect anti-lung cancer activity
The pancreatic cancer cell line BX-PC-3 was used to detect anti-pancreatic cancer activity.
Cell lines were purchased from American Type Culture Collection (ATCC).
   Cell Culture conditions:

The cancer cell cultures were grown as recommended by the ATTC manuals. A549 cells and BX-PC-3 cells were grown in DME Dulbecco's modified Eagle's medium containing 4500 mg/L glucose; 4 mM L-glutamine; 10 U/ml Pen-G; 10 mcg/ml medium and 10% fetal calf serum (FCS). PC-3 and MDA-MB468 cells were grown in RPMI medium 1640 containing 2 mM L-glutamine; 10 U/ml Pen-G; 10 mcg/ml Streptomycin and 10% FCS. Cells were kept at 6% CO₂ and 37° C. Cells were seeded on day zero in 96-well format tissue culture plates at suitable densities the day before starting treatment, in the media indicated above.

Treatment of Cancer Cells With The Compounds:

On day one, the compounds of the invention were added to the culture media of growing cells, containing 10% FQS. The cell media contained the compounds of the invention at one of six concentrations: 1×10⁻⁸, 5×10⁻⁸, 1×10⁻⁷, 5×10⁻⁷, 1×10⁻⁶, and 1×10⁻⁵M. In control experiments, 0.1% DMSO was used as vehicle control, and never exceeded 0.1% final concentration. On day four the media was removed from the cells and replaced with fresh media containing the compounds of the invention and FCS at the appropriate concentrations.

MTT Assay Procedure:

On day five 10 μl of 5 mg/ml MTT dye was added to each well containing a cell culture. The MTT assay is based on the dehydrogenase activity in active mitochondria for cleavage of the yellow tetrazolium salt MTT to produce purple formazan crystals. This conversion of MTT only occurs in living cells with intact/functional mitochondria. After addition of MTT, the cells were incubated for additional 4 hours at 6% CO₂ and 37° C. Reaction was then stopped by adding 100 μl/well of a solubilization solution consisting of 10% Sodium Dodecyl Sulfate (SDS) and 10 mM HCl. On day 6 the formazan crystals formed were solubilized and the resulting colored solution quantified using a scanning multiwell spectrophotometer at a wavelength of 595 nm.

Selected results of the screening experiments for compounds 1-3 and 5-14, are shown in FIGS. 7-10. The chemical structure and method of synthesis for compounds 1-3 and 5-14 is described in Examples 1-3 and 5-14.

### Example 22

### Comparative In vitro Testing of Cancer Drug Candidates in Human Cancer Cell Based Assays

The procedure of Example 21 was employed to measure the anti-cancer activity of compounds 1 & 2 of the invention and compare them with equivalent activity tests for Comparative Compound 4, whose synthesis is given in Example 4. Comparative compound 4 is analogous to Compounds 1 and 2, but comprises a methylenedioxy ring on its “Ar₁” radical, rather than the benzoxazole, benzothiazole, or benzimidazole ring that is present in the compounds described and claimed herein.

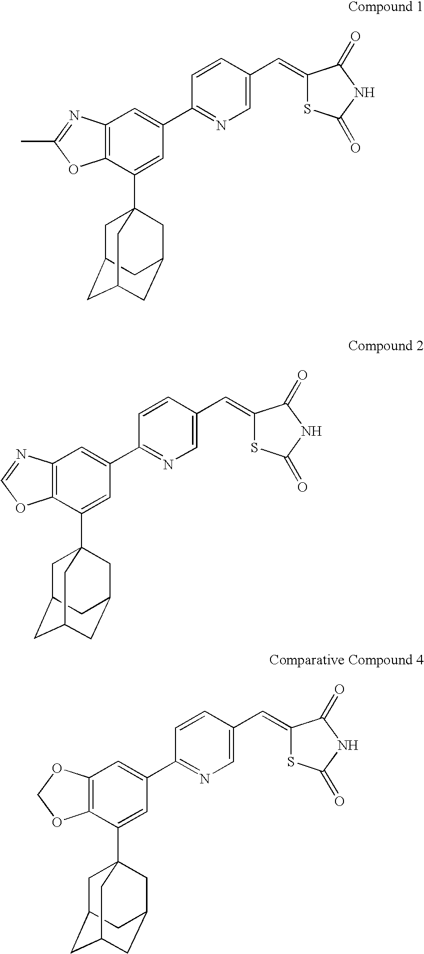

The results of the comparative activity testing are shown in FIGS. 11-14. As can be seen in the Figures, all three compounds when administered in concentrations in the range of 10⁻⁷-10⁻⁵ M or higher, kill significant percentages of the cells of breast cancer, prostate cancer, lung cancer, and pancreatic cancer cultures. Nevertheless, as is unexpectedly apparent from FIGS. 11-14, Compounds 1 and 2 were active to inhibit cancer cell growth and/or induce cancer cell apoptosis at concentrations that are a factor of 5-10 lower than the concentrations that Comparative Compound 4, which differs only by the structure of the non-aromatic methylenedioxy heterocyclic ring.

### Example 23

### In vitro Screening for JNK-activation of Cancer Drug Candidates

An indication that the compounds disclosed herein activate the JNK cell signaling pathways associated with cell apoptosis has been demonstrated by in vitro experiments involving treating a lung cancer cell line with compounds 1, 2, 11, and 12 of the invention, followed by Western Blotting assays for activated (phosphorylated) JNK proteins. Phosphorylated JNK proteins can be specifically detected by employing an antibody specific to phosphorylated JNK, followed by Western Blotting analysis. The JNK phosphorylation induced was compared to that of control/untreated tumor cells, which did not exhibit significant levels of phosphorylated JNK proteins. In particular, the human lung cancer cell line H292, purchased from the American Type Culture Collection (ATCC) (Manassas, Va.), was tested for JNK-activation induced by compounds 1, 2, 11, and 12 described herein.

Culture Conditions:

H292 cells were grown in RPMI medium 1640 containing 2 mM L-glutamine; 10 U/ml Pen-G; 10 mcg/ml Streptomycin and 10% FCS.

Cells were kept at 6% CO₂ and 37° C. H292 cells were plated at 70% confluence (adherent growing cells covering 70% of culture plate surface area) in a 10 centimeter tissue culture dish in the medium indicated above.

Treatment:

Compounds 1, 2, 11, and 12 were applied to cultures of the H292 cells in the medium indicated above at a concentration of 2.5 micromolar. DMSO (dimethyl sulfoxide, Sigma, St. Louis, Mo.) was used as vehicle control, and never exceeded 0.1% final concentration. Treatment was for 16 hours.

Western Blot Assay:

At the end of incubation of the cultured cells with the test compounds, the medium was removed and the plated cells were washed twice with cold PBS (phosphate buffer saline). Excess PBS was aspirated away and the cells were lysed and scraped into sample buffer containing 50 mM HEPES pH 7.5 (buffer), 150 mM NaCl, 0.1% Tween 20 (a detergent, Biorad, Hercules, Calif.), 20 mM NaF, 10 mcg/mL aprotinin, 10 mcg/mL leupeptin. Samples were incubated on ice for 15 minutes and insoluble material was pelleted by microfugation. Protein concentrations for each sample were determined using BSA (Bovine serum albumin, Sigma, St Louis, Mo.) as a standard in a colorimetric protein quantification assay (BioRad, Hercules, Calif.).

Procedure:

100 mcg of each sample of cellular lysate were subjected to electrophoresis on 12% SDS-PAGE (polyacrylamide gel electrophoresis) gels (BioRad, Hercules, Calif.). Proteins were transferred to PVDF membrane. Membranes were probed with a monoclonal antibody recognizing phosphorylated JNK (Cell Signaling, Beverly, Mass.) followed by HRP(horseradish peroxidase)-conjugated goat-anti-mouse antibody (Santa Cruz Biotechnology, Santa Cruz, Calif.). Immunoreative bands were visualized by ECL (enhanced chemiluminescence, Amersham, Buckinghanshire, England) detection on film (Kodak, Rochester, N.Y.).

Results:

As shown in FIG. 15, treatment of the cancer cells with compounds 1 and 2 induced the phosphorylation of JNK proteins, as shown in the upper panel by the phospho-JNK bands (representing two isoforms of activated JNK) present in the compound 1 and 2 lanes, but not in the control-treated lane. Compound 12 activates the phosphorylation of JNK proteins, though perhaps less potently than compound 1 or 2, as deduced from the reduced intensity of the phospho-JNK band. Compound 11 only weakly induced the phosphorylation of JNK proteins, as shown by the weak lower band in the phospho-JNK panel, and corresponding showed only relatively weak activity against a different line of human lung cancer cells, as shown in FIG. 7.

As a control experiment, the same blot was probed with an antibody that recognizes all isoforms of JNK, activated or not (lower panel). This blot shows that a number of unphosphoryla'ted JNK proteins are present in all the samples. Thus, a failure to detect activated or phosphorylated JNK, as in the control lane, is due to a lack of JNK activation, not due to an absence of JNK.

Therefore, although not wishing to be bound by any mechanism or theory of action or effectiveness, these experiments provide evidence that Compounds 1 and 2 are potent activators of the phosphorylation of JNK proteins in H292 cells.

Throughout this application, various publications are referenced. The disclosures of these publications in their entireties are hereby incorporated by reference into this application.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of the formula 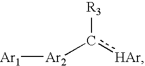
wherein:
a) Ar₁ has the structure: 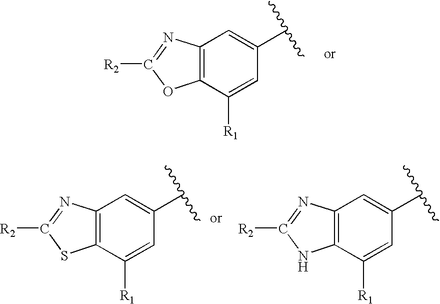
wherein
i) R₁ has the structure 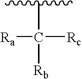
and wherein Rₐ, R_{b}, and R_{c} are independently selected from hydrogen, alkyls, and substituted alkyls, wherein two or three of the Rₐ, R_{b}, and R_{c} radicals can optionally together form cyclic, bicyclic, or polycyclic cycloalkyl or heterocyclic rings, with the proviso that no more than one of Rₐ, R_{b}, and R_{c} are hydrogen, and that Rₐ, R_{b}, and R_{c} together comprise between 3 and 11 carbon atoms;
ii) R₂ is selected from the group consisting of hydrogen, amino, or a monosubstituted amino, disubstituted amino, alkoxy, or alkyl radical having 1 to 4 carbon atoms;
b) Ar₂ has the structure; 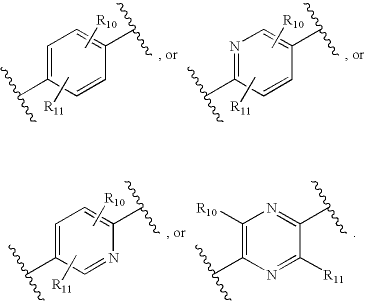
wherein the R₁₀ and R₁₁ substituent radicals are independently selected from hydrogen, hydroxyl, amino, halogen, or organic radicals comprising 1 to 4 carbon atoms independently selected from alkyl, alkoxy, haloalkyl, and haloalkoxy radicals;
c) R₃ is hydrogen, or an alkyl radical comprising 1 to 4 carbon atoms;
d) [symbol] represents a bond present or absent; and
e) HAr has the formula 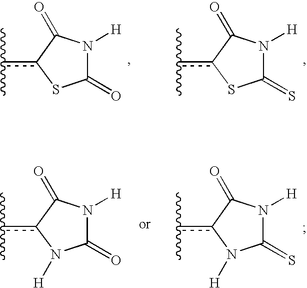
or a pharmaceutically acceptable salt thereof.

2. The compounds of claim 1 wherein Rₐ, R_{b}, and R_{c} are independently selected alkyls.

3. The compounds of claim 1 wherein two or three of the Rₐ, R_{b}, and R_{c} radicals together form cyclic, bicyclic, or polycyclic cycloalkyl or heterocyclic rings.

4. The compounds of claim 1 wherein R₁ has the structure

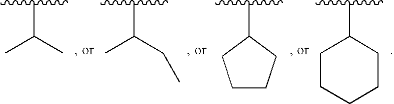

5. The compounds of claim 1 wherein R₁ has the structure

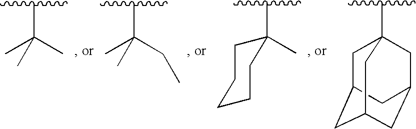

6. The compounds of claim 1 wherein R₁ has the structure

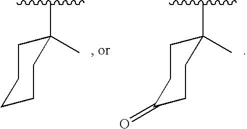

7. The compounds of claim 1 wherein R₁ has the formula

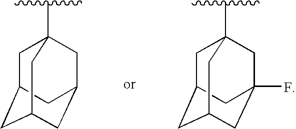

8. The compounds of claim 1 wherein R₁ has the formula

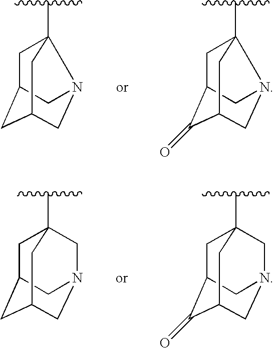

9. The compounds of claim 1 wherein Ar₁ has the formula

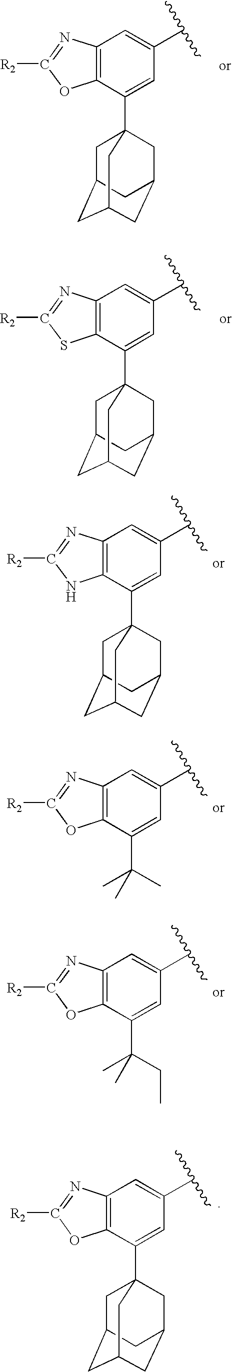

10. The compounds of claim 1 wherein R₂ is selected from the group consisting of hydrogen, amino, methyamino, dimethylamino, methoxy, or methyl.

11. The compounds of claim 1 wherein Ar₂ has the formula

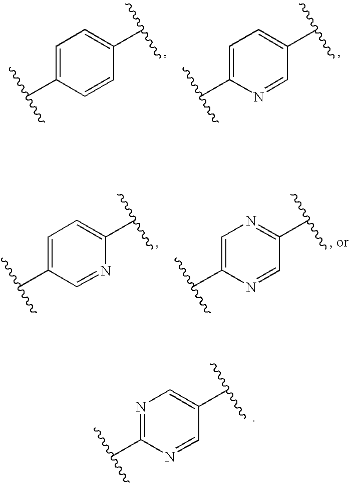

12. The compounds of claim 9 wherein Ar₂ has the formula

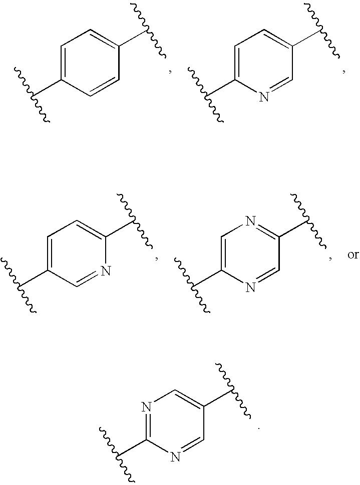

13. The compounds of claim 1 wherein R₃ is hydrogen.

14. The compounds of claim 12 wherein R₃ is hydrogen.

15. The compounds of claim 1 wherein ----- represents a bond is present.

16. The compounds of claim 1 wherein HAr has the formula

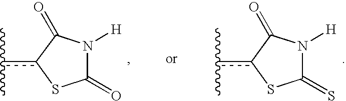

17. A pharmaceutical composition comprising one or more of the compounds of claim 1 or pharmaceutically acceptable salt or prodrug thereof, and one or more pharmaceutically acceptable carriers.

18. A method for the treatment of a disease of uncontrolled cellular proliferation comprising administering to a mammal diagnosed as having a disease of uncontrolled cellular proliferation one or more compounds of claim 1 or a pharmaceutically acceptable salt or prodrug thereof or a pharmaceutical composition thereof, in an amount effective to treat the disease of uncontrolled cellular proliferation.

19. The method of claim 18 wherein the disease of uncontrolled proliferation is a carcinoma, lymphoma, leukemia, or sarcoma.

20. The method of claim 18 wherein the disease of uncontrolled proliferation is a cancer.

21. The method of claim 20 wherein the cancer is Hodgkin's Disease, meyloid leukemia, polycystic kidney disease, bladder cancer, brain cancer, head and neck cancer, kidney cancer, lung cancer, myeloma, neuroblastoma/glioblastoma, ovarian cancer, pancreatic cancer, prostate cancer, skin cancer, liver cancer, melanoma, colon cancer, cervical carcinoma, breast cancer, epithelial cancer, or leukemia.

22. The method of claim 20 that additionally comprises administration of one or more known therapeutic agents that are effective for the treatment of cancer.

23. A compound of the formula:
5-[6-(7-Adamantan-1-yl-2-methyl-benzoxazol-5-yl)-pyridin-3-ylmethylene]-thiazolidine-2,4-dione;
5-[6-(7-Adamantan-1-yl-benzoxazol-5-yl)-pyridin-3-ylmethylene]-thiazolidine-2,4-dione;
5-[6-(7-Adamantan-1-yl-2-phenyl-benzoxazol-5-yl)-pyridin-3-ylmethylene]-thiazolidine-2,4-dione;
5-[4-(7-Adamantan-1-yl-2-methyl-benzoxazol-5-yl)-benzylidene]-thiazolidine-2,4-dione;
5-[3-(7-Adamantan-1-yl-2-methyl-benzoxazol-5-yl)-benzylidene]-thiazolidine-2,4-dione;
5-[4-(5-Adamantan-1-yl-2-methyl-benzoxazol-7-yl)-benzylidene]-thiazolidine-2,4-dione;
5-[4-(5-Adamantan-1-yl-2-methyl-benzoxazol-7-yl)-benzylidene]-2-thioxo-thiazolidin-4-one;
5-[3-(5-Adamantan-1-yl-2-methyl-benzoxazol-7-yl)-benzylidene]-thiazolidine-2,4-dione;
-[3-(5-Adamantan-1-yl-2-methyl-benzooxazol-7-yl)-benzylidene]-2-thioxo-thiazolidin-4-one;
5-[6-(7-Cyclohexyl-2-methyl-benzoxazol-5-yl)-pyridin-3-ylmethylene]-thiazolidine-2,4-dione;
5-[6-(7-Cyclohexyl-benzoxazol-5-yl)-pyridin-3-ylmethylene]-thiazolidine-2,4-dione;
5-[6-(7-Cyclohexyl-2-trichloromethyl-benzoxazol-5-yl)-pyridin-3-ylmethylene]-thiazolidine-2,4-dione;
5-[6-(7-Adamantan-1-yl-2-amino-benzoxazol-5-yl)-pyridin-3-ylmethylene]-thiazolidine-2,4-dione;
5-{6-[7-(1,1-Dimethyl-propyl)-benzoxazol-5-yl]-pyridin-3-ylmethylene}-thiazolidine-2,4-dione;
5-{6-[7-(1,1-Dimethyl-propyl)-2-methyl-benzooxazol-5-yl]-pyridin-3-ylmethylene}-thiazolidine-2,4-dione;)
N-{7-Adamantan-1-yl-5-[5-(2,4-dioxo-thiazolidin-5-ylidenemethyl)-pyridin-2-yl]-benzooxazol-2-yl}-2,2,2-trifluoro-acetamide;
N-{7-Adamantan-1-yl-5-[5-(2,4-dioxo-thiazolidin-5-ylidenemethyl)-pyridin-2-yl]-benzooxazol-2-yl}-acetamide;
5-[6-(7-Benzyloxy-benzoxazol-5-yl)-pyridin-3-ylmethylene]-thiazolidine-2,4-dione; or
5-[6-(7-Benzyloxy-2-methyl-benzoxazol-5-yl)-pyridin-3-ylmethylene]-thiazolidine-2,4-dione;
or a pharmaceutically acceptable salt thereof.
